(19) United States
(12) Patent Application Publication
(10) Pub. No.: US 2026/0326102 A1
(43) Pub. Date: Oct. 22, 2026

(54) CENTRIFUGAL MICROFLUID CHIP FOR NUCLEIC ACID DETECTION AND ITS DETECTION METHOD

(71) Applicant: SUZHOU XIANDA GENE TECHNOLOGY CO., LTD., Suzhou (CN)

(72) Inventors: Wenzhu CHEN, Suzhou (CN); Danni LIU, Suzhou (CN); Jibin YU, Suzhou (CN); Jianfeng WANG, Suzhou (CN); Jiang LIU, Suzhou (CN)

(21) Appl. No.: 19/687,177

(22) Filed: May 26, 2026

Related U.S. Application Data
(63) Continuation-in-part of application No. PCT/CN2024/094165, filed on May 20, 2024.

(30) Foreign Application Priority Data
Dec. 14, 2023 (CN) 202311719955.8
Dec. 27, 2023 (CN) 202323587206.2
Dec. 27, 2023 (CN) 202323593781.3
Apr. 7, 2024 (CN) 202410409574.8
Apr. 26, 2024 (CN) 202420887613.0

(51) Int. Cl. B01L 3/00

(52) U.S. Cl. CPC B01L 3/50273; B01L 3/502715; B01L 2200/027; B01L 2200/0684; B01L 2200/10; B01L 2200/16; B01L 2300/0816; B01L 2300/087; B01L 2300/0883; B01L 2400/0409

(57) ABSTRACT

The present invention provides A microfluidic chip for detecting a nucleic acid, comprising: a reaction chamber configured for amplifying target nucleic acid in a liquid sample to obtain an amplification product; a detection chamber configured for detecting the target nucleic acid in the amplification product; the reaction chamber is in liquid communication with the detection chamber by a first liquid channel, a second channel for receiving the liquid sample including inlet and outlet; the detection chamber is in liquid communication with the second channel by a third channel; wherein air exhaust channel with a first end and a second end, the first end of the exhaust channel is air communication with the detection chamber or/and the reaction chamber, the second end of the exhaust channel is air communication with the second channel.

### CROSS-REFERENCE TO RELATED APPLICATIONS

This application is continuation in part of the PCT application with the filing no. PCT/CN2024/094165 with the filing date of May 20, 2024 that claims priority to Chinese Application No. 202311719955.8, filed on Dec. 14, 2023; 202323587206.2, filed on Dec. 27, 2023; 202323593781.3, filed on Dec. 27, 2023; 202410409574.8, filed on Apr. 7, 2024 and 202420887613.0, filed on Apr. 26, 2024. The specifications, abstracts, drawings, and claims of the applications are incorporated as part of this application.

### BACKGROUND OF INVENTION

### Field of the Invention

The present invention relates to the field of microfluidic control, and specifically relates to a centrifugal microfluidic chip for nucleic acid detection and a detection method thereof.

### Description of the Related Art

Microfluidic chip technology, also known as lab-on-a-chip, can integrate or basically integrate basic operations involved in fields such as biology and chemistry, such as sample preparation, biological and chemical reactions, separation and detection, onto a chip of several square centimeters to complete different biological or chemical reaction processes and analyze their products. This technology can greatly reduce the requirements of the detection process on sites, personnel, and equipment.

Existing microfluidic detection chips have integrated multiple steps such as nucleic acid amplification and nucleic acid detection on a small chip, and can quickly complete the detection process of “sample in—result out”. Since microfluidic chips are not dependent on precision pipetting devices and professional personnel, they have great potential in the field of point-of-care testing (POCT).

Isothermal amplification has attracted more and more attention in the field of POCT due to its fast reaction speed and low requirements for instruments and equipment. However, due to the complexity of samples to be tested, such as blood, sputum, urine, and tissues, after isothermal amplification, it is usually necessary to react with detection reagents to amplify signals to achieve accurate detection, which is called nucleic acid multiplex detection. To realize nucleic acid multiplex detection, a microfluidic chip must be equipped with both a reaction chamber and a detection chamber. The reaction chamber is used for amplification reaction, and the detection chamber is used for signal amplification. In addition, the sample to be tested needs to be controllably transferred between the reaction chamber and the detection chamber, and sample backflow must be prevented to avoid cross-influence. Most of the existing microfluidic chips only have one reaction chamber for nucleic acid amplification and detection, making it difficult to realize nucleic acid multiplex detection.

At the same time, existing microfluidic chips generally require external pressure devices, centrifugal force, or siphonage as driving forces. The use of external pressure devices, such as negative pressure vacuum pumps and syringe pumps, has strong driving force, but they need to be connected to the chip externally, making the detection platform structure complex. Using centrifugal force as the driving force has the problem of relying on electricity and centrifugal equipment. Although the siphon-driven liquid flow method does not require equipment, its driving force is small, and there is a problem of volatilization during sample injection. In order to realize nucleic acid multiplex detection, there are also methods to control multiplex detection through external pressure supply devices, but the operation process is complex and has high requirements. For example, the patent with publication number CN113736643 provides a microfluidic chip capable of realizing nucleic acid multiplex detection, but it requires an external pressure supply device for sample injection. A hydrophobic valve and a membrane valve are also specially arranged between the reaction amplification chamber and the detection chamber. The external pressure provides positive pressure, the membrane valve and the hydrophobic valve are closed; when the positive pressure is released, the membrane valve and the hydrophobic valve are opened. At the same time, an electromagnetic valve is required to control the external pressure supply device, and the structure of the microfluidic chip is complex and the cost is high, which greatly limits the use of the microfluidic chip in environments with limited resources.

Centrifugal microfluidic chips drive the flow of liquid by rotating the centrifugal microfluidic chip, and can realize the manipulation of liquid at the sub-millimeter scale using centrifugal force. Compared with traditional microfluidic chips, the entire device is more compact and simpler to handle, so it is gradually applied in the field of diagnosis. In centrifugal microfluidic chips, microchannels are distributed along the radial direction of the disc. When the chip rotates, the liquid to be tested moves to each reaction pool along multiple microchannels under the action of centrifugal force, realizing the distribution and sample addition of the liquid to be tested.

Current diagnostic technologies usually require multiple indicators to determine whether a patient has a certain disease. For example, in the clinical testing process, the same patient sample needs to be screened for multiple respiratory pathogens, and these indicators vary according to individual conditions. However, high-throughput centrifugal microfluidic chips also have problems such as short and inaccurate control processes and complex structures. When detecting multiple indicators at the same time, each chamber in the centrifugal microfluidic chip is prone to uneven sample injection volume, and may enter other chambers during centrifugation, or backflow may easily occur after centrifugation, leading to mutual interference and pollution. As a result, existing centrifugal microfluidic chips can only perform simultaneous amplification of single samples or a small number of detection indicators. For example, the nucleic acid multiplex detection microfluidic chip provided by the patent with publication number CN220166182U has low detection throughput and is difficult to complete the screening of samples and multi-indicator pathogens in a short time. When facing the amplification needs of multiple samples and multiple indicators, it not only cannot provide efficient and fast amplification work, but also faces high chip replacement costs.

Patent CN115684014A provides a microfluidic chip for centrifugation, which can also realize high-throughput detection of multiple indicators, but its structure and operation process are relatively complex. Vent holes are arranged on the bottom plate and the cover plate for ventilation of the entire chip. Samples enter the corresponding chambers through centrifugation. Since it is difficult to control the amount of sample entering the chambers during each centrifugation, a transition pool is also required to measure the sample volume using the volume of the transition pool. Therefore, it requires three-stage centrifugation to make the liquid reach the target position for detection (first-stage centrifugation to the transition pool, second-stage centrifugation to the reaction chamber, and third-stage centrifugation to the detection chamber). Moreover, since the centrifugal speed of the latter stage must be significantly higher than that of the previous stage to make the liquid enter the next chamber, the three-stage centrifugation inevitably requires higher centrifugal force. This not only makes the operation more cumbersome, but also the pre-embedded freeze-dried or vitrified reagents are easily precipitated due to insufficient dissolution during high-speed centrifugation, leading to reaction failure and seriously affecting the detection results. At the same time, the chip has many holes, and the flow channels and holes are very small, making it difficult to control the tolerance, and the liquid is prone to misalignment and loss of control during sample addition.

Therefore, there is an urgent need to find a nucleic acid multiplex detection microfluidic chip with simpler structure, more convenient operation, lower instrument requirements, and lower manufacturing cost. At the same time, for centrifugal nucleic acid multiplex detection microfluidic chips, it is expected to realize that the sample can accurately enter the corresponding chambers with only one or two centrifuging steps, and the amount of sample entering each chamber can be effectively controlled. There should be no sample backflow or leakage into other chambers, so as to completely solve the problems of mutual interference and pollution. Moreover, it can perform high-throughput detection of multiple samples and multiple detection items, and ensure the accuracy of detection results.

### SUMMARY OF THE INVENTION

To solve the above problems, the present invention provides a nucleic acid detection microfluidic chip and its detection method, including a nucleic acid multiplex detection microfluidic chip. The nucleic acid multiplex detection microfluidic chip realizes the controllable transfer of samples between the reaction chamber and the detection chamber by arranging a fluid channel between the reaction chamber and the detection chamber, and making the reaction chamber and the detection chamber share an exhaust channel. The combination of the fluid channel and the exhaust channel can automatically provide resistance for the sample in the reaction chamber to enter the detection chamber. On the basis of ensuring detection precision, the structure and operation process are greatly simplified, thus designing a gravity-driven microfluidic chip and a centrifugal microfluidic chip.

The gravity-driven microfluidic chip has a multi-turn tortuous fluid channel, and the pore size of the fluid channel is smaller than that of the sample inlet channel of the reaction chamber. This enables the sample to flow from the sample-adding area into the reaction chamber under the action of gravity, but cannot flow into the fluid channel. After applying pressure through a simple pressure device, the sample can smoothly enter the detection chamber from the reaction chamber through the fluid channel, realizing the controllable transfer of the sample in different chambers and effectively preventing sample backflow. The structure is simple: only an exhaust channel and a waterproof and breathable membrane need to be arranged in the detection chamber, and no exhaust channel is needed in the reaction chamber. The transfer of the sample in different chambers can be realized only by gravity or a simple pressure device on the chip, without additional driving equipment.

The centrifugal microfluidic chip has an exhaust channel leading out from the fluid channel between the reaction chamber and the detection chamber, with the exhaust direction opposite to the rotational centrifugal direction, and the exhaust port is positioned higher than the fluid channel. Combining the principle of internal and external atmospheric pressure balance and centrifugal driving performance, it can effectively control the flow of gas and liquid in the reaction chamber and the detection chamber, and effectively control the amount of sample entering each chamber. Nucleic acid multiplex detection can be completed with only one or two centrifugations. The sample to be tested can be stably retained and reacted after entering each chamber, effectively preventing sample backflow and eliminating the problems of mutual interference or pollution. At the same time, by arranging one or more concentric sub-chips outside the chip body, high-throughput detection of more samples and more detection items can be realized, and the detection efficiency can be improved.

At the same time, the present invention also provides a nucleic acid typing detection microfluidic chip. This chip is only provided with reaction chambers and no detection chambers. Multiple reaction chambers are connected to the liquid storage chamber, and the exhaust channels of the reaction chambers are connected to the liquid storage chamber. Gravity-driven typing chips and asymmetric pressure-driven typing chips are designed to effectively avoid aerosol contamination.

On one hand, the present invention provides a nucleic acid detection microfluidic chip. The chip is provided with a reaction chamber and a detection chamber; the reaction chamber is used for amplifying the target nucleic acid in the sample to obtain an amplification product; the detection chamber is used for detecting the target nucleic acid in the amplification product; The reaction chamber and the detection chamber are connected through a fluid channel, which is used for realizing the circulation of liquid or gas between the reaction chamber and the detection chamber; The reaction chamber and the detection chamber share an exhaust channel, which is used for controlling the flow of gas or liquid in the reaction chamber or the detection chamber; The exhaust channel is connected to the fluid channel or the detection chamber; the combination of the fluid channel and the exhaust channel can automatically provide resistance for the sample in the reaction chamber to enter the detection chamber.

In some embodiments, the microfluidic chip provided by the present invention is mainly used for nucleic acid multiplex detection. The nucleic acid multiplex detection means that nucleic acid needs to be detected in two steps: in the first step, the sample enters the reaction chamber, and the target nucleic acid in the sample is amplified in the reaction chamber; in the second step, after the amplification is completed, the amplification product enters the detection chamber. Therefore, the microfluidic chip must realize the controllable transfer of the sample between the reaction chamber and the detection chamber, and prevent cross-influence caused by sample backflow. The controllable transfer of the sample between the reaction chamber and the detection chamber means that when the sample enters the reaction chamber, all the samples to be tested smoothly enter the reaction chamber and remain in the reaction chamber for amplification reaction; after the amplification reaction is completed, the amplification product flows out of the reaction chamber, enters the detection chamber through the fluid channel, and remains in the detection chamber to complete the detection reaction until the final smooth detection.

Existing nucleic acid multiplex detection microfluidic chips usually need to arrange a valve between the reaction chamber and the detection chamber for blocking, so that the sample will not enter the detection chamber when entering the reaction chamber.

In some embodiments, a valve block can also be added between the reaction chamber and the detection chamber to control the transfer of the sample between the reaction chamber and the detection chamber. When the valve block (paraffin) is solid, the reaction chamber and the detection chamber cannot be in fluid communication; when the valve block is liquid, the detection chamber and the reaction chamber are in fluid communication. Using the temperature-induced phase change property of the valve block and the characteristics of the waterproof and breathable membrane, combined with the principle of internal and external atmospheric pressure balance, the controllable transfer of the sample to be tested in different chambers is cleverly realized. In this way, both the reaction chamber and the detection chamber need to be provided with exhaust channels and waterproof and breathable membranes. Moreover, the arrangement of the valve block complicates the preparation process of the microfluidic chip, and the heating temperature needs to be controlled to control the state of the valve block, which adds additional steps to the operation process.

The combination of the fluid channel and the exhaust channel in the present invention refers to the combination of the design of various characteristics of the fluid channel (quantity, length, shape, height position, pore size, etc.) and the design of various characteristics of the exhaust channel (quantity, distance from the detection chamber, position, design of the exhaust port, direction of the exhaust channel, pore size, etc.).

The present invention realizes the controllable transfer of the sample between the reaction chamber and the detection chamber by improving the structure and positional relationship of the fluid channel and the exhaust channel to automatically provide resistance for the sample in the reaction chamber.

When the reaction chamber and the detection chamber share one exhaust channel, the structure and position of the exhaust channel mainly include the distance between the exhaust channel and the detection chamber, or the direction of the exhaust channel, etc. It can be understood that the distance between the exhaust channel and the detection chamber is directly related to the resistance of the sample entering the detection chamber. Only when the exhaust channel is connected to the top of the detection chamber, when the liquid flows from the reaction chamber to the detection chamber and enters the detection chamber from the bottom of the detection chamber, the exhaust direction is also from the bottom of the detection chamber to the top of the detection chamber and then discharged from the exhaust channel. The exhaust direction is consistent with the liquid flow direction, so the resistance of the liquid sample entering the detection chamber is the smallest; when the exhaust channel is connected to other parts of the detection chamber, or not connected to the detection chamber but connected to the fluid channel, the discharged gas will be discharged from the bottom of the detection chamber to the top, then turned back, and pass through the liquid to reach the exhaust channel. That is to say, part of the exhaust process is opposite to the liquid flow direction. This turning back and passing through the liquid process will significantly increase the resistance of the sample entering the detection chamber. Therefore, changing the position of the exhaust channel will directly affect the effect of the controllable transfer of the sample between the reaction chamber and the detection chamber.

Improving the structure of the fluid channel between the reaction chamber and the detection chamber will also directly affect the resistance of the sample entering the detection chamber from the reaction chamber. For example, the longer the length of the fluid channel, the more complex the structure (more tortuous or folded structures), the higher the height than the reaction chamber, and the smaller the pore size, etc., will all increase the difficulty of the sample in the reaction chamber entering the detection chamber, so that the fluid channel can automatically provide resistance for the sample in the reaction chamber, making it difficult for the sample in the reaction chamber to automatically enter the detection chamber. The sample can only enter the detection chamber after a certain pressure (a simple pressure device on the chip) is applied or centrifugation is performed.

The gravity-driven microfluidic chip provided by the present invention is provided with multiple groups of detection units. Each group of detection units is arranged in multiple levels on the chip body. Each group of detection units is provided with a reaction chamber and a detection chamber, which can be applied to the simultaneous detection of multiple different target analytes, such as the detection of multiple different nucleic acid targets, and each group of detection units can realize nucleic acid multiplex detection. Of course, it can be understood that the gravity-driven microfluidic chip is not only used for nucleic acid multiplex detection, but also for the detection of other molecules such as proteins and antibodies. The reaction chamber can be used for sample pretreatment, and the detection chamber can be used for sample detection.

Further, the chip includes a gravity-driven microfluidic chip and/or a centrifugal microfluidic chip; when the chip is a gravity-driven microfluidic chip, the fluid channel can automatically provide resistance for the sample in the reaction chamber, making it difficult for the sample in the reaction chamber to automatically enter the detection chamber.

The gravity-driven microfluidic chip refers to a chip where the sample can directly reach the reaction chamber from the sample area under the action of gravity without any external driving force. For example, when used for nucleic acid multiplex detection, the sample can directly reach the reaction chamber from the sample area under the action of gravity, but when the sample enters the reaction chamber, the fluid channel needs to act as a valve to prevent the sample from entering the detection chamber at the same time. Through the structural design of the fluid channel, the fluid channel can provide resistance for the sample to enter the detection chamber from the reaction chamber, thereby acting as an automatic valve. The structural design of the fluid channel mainly includes setting the length, structure, pore size, height, inner wall roughness of the fluid channel, etc., to provide resistance for the sample to enter the detection chamber from the reaction chamber.

In some embodiments, the fluid channel can also be designed by combining any one, two, three, or four of the length, structure, pore size, and height of the fluid channel to automatically provide resistance for the sample to enter the detection chamber from the reaction chamber. Some preferred methods can also be selected to increase the controllability of the resistance provided by the fluid channel and improve the effect of the fluid channel acting as a valve.

Further, the length of the fluid channel is longer than the straight-line distance between the reaction chamber and the detection chamber.

It can be understood that when the length of the fluid channel is increased, the resistance of the sample entering the detection chamber from the reaction chamber through the fluid channel will also increase. There are many ways to increase the length of the fluid channel, such as increasing the degree of bending, repeating the bending multiple times, making the path go around multiple times, extending in different directions, or increasing the height span of local or multiple points in the fluid channel, etc. All these ways can increase the length of the fluid channel and increase the resistance of the sample entering the detection chamber from the reaction chamber. These methods can be used alone or in combination of any kind. According to the required resistance, a certain screening and optimization experiment can be carried out to find a more preferred and suitable fluid channel design method. For example, while increasing the degree of bending, increasing the number of repeated bends; while increasing the degree of bending, increasing the height span, etc., so as to improve the controllability of the resistance provided by the fluid channel and improve the effect of the fluid channel acting as a valve.

Further, the fluid channel is a folded, spiral, or multi-turn tortuous flow channel, and the folded or multi-turn tortuous flow channel is provided with at least one bent structure.

When the fluid channel is designed to have multiple folded, spiral, or multi-turn tortuous structures, the length of the fluid channel can be extended as much as possible, and the difficulty of the sample in the reaction chamber entering the fluid channel without additional pressure can be maximized, but it provides a guarantee for all the samples in the reaction chamber to remain in the reaction chamber.

Further, the fluid channel extends toward the upper end of the reaction chamber, the upper end of the detection chamber, or directly above the fluid channel.

It can be understood that the extension of the fluid channel in any direction will increase the length of the fluid channel or increase the degree of tortuous folding, thereby increasing the difficulty of the sample entering the detection chamber from the reaction chamber through the fluid channel without additional pressure.

Further, the position of the detection chamber is higher than that of the reaction chamber, or the position of the sample inlet of the detection chamber is higher than that of the sample outlet of the reaction chamber, or the bottom of the detection chamber is not lower than the top of the reaction chamber.

During the use of the gravity-driven microfluidic chip, it needs to be kept in a vertical state, with the sample inlet facing upward. The following description of the positional relationship of each flow channel and chamber in the gravity-driven microfluidic chip is based on the gravity-driven microfluidic chip being kept in a vertical state. In some embodiments, the gravity-driven microfluidic chip can be inserted into a matching detection device, so that the gravity-driven microfluidic chip is placed vertically with the sample inlet facing upward, and the detection device can provide a heat source to heat specific positions of the gravity-driven microfluidic chip (such as the reaction chamber, valve block, etc.); in some embodiments, the detection device can also detect the fluorescent substances generated after the sample reaction in the gravity-driven microfluidic chip and read the detection results.

In some embodiments, the positional relationship of the gravity-driven microfluidic chip of the present invention can refer to the height relationship of each chamber when it is kept in a vertical state. Of course, this does not mean that the fluid channel can only play the role of providing resistance for the sample to enter the detection chamber from the reaction chamber when the gravity-driven microfluidic chip is kept in a vertical state. In fact, whether the gravity-driven microfluidic chip is in a horizontal, inclined, or vertical state, it can play the role of providing resistance for the sample to enter the detection chamber from the reaction chamber.

It can be understood that when the gravity-driven microfluidic chip is kept in a vertical state, the higher the position of the detection chamber relative to the reaction chamber, the greater the difficulty of the sample entering the detection chamber from the reaction chamber under the action of gravity, and the sample cannot enter the detection chamber under the action of gravity.

In some embodiments, the sample flows out from the sample outlet of the reaction chamber, passes through the fluid channel, and then enters the sample inlet of the detection chamber. Therefore, if the position of the sample inlet of the detection chamber is higher than that of the sample outlet of the reaction chamber, the difficulty of the sample entering the detection chamber from the reaction chamber under the action of gravity will also increase, and the sample cannot enter the detection chamber under the action of gravity.

Further, at least one part of the fluid channel is higher than the reaction chamber.

In some embodiments, when the gravity-driven microfluidic chip is kept in a vertical state, a section of the fluid channel or a point is higher than the reaction chamber, which can also increase the difficulty of the sample entering the detection chamber from the reaction chamber under the action of gravity, and the sample cannot enter the detection chamber under the action of gravity.

In some embodiments, a section of the fluid channel or a point is higher than the sample outlet of the reaction chamber.

Further, the fluid channel includes at least one arched flow channel, and the arched flow channel includes a sample inlet section, a middle section, and a sample outlet section. The middle section is arched upward, the position of the middle section is higher than that of the sample inlet section and the sample outlet section, and at least the position of the middle section is higher than that of the reaction chamber.

The upwardly arched middle section of the fluid channel increases the difficulty of the sample in the reaction chamber entering the fluid channel without additional pressure, which helps all the samples in the reaction chamber remain in the reaction chamber to complete the nucleic acid amplification reaction.

Further, it further includes a sample-adding area; a sample inlet channel is arranged between the sample-adding area and the reaction chamber; the pore size of the fluid channel is smaller than that of the sample inlet channel.

In some embodiments, the pore size of the fluid channel is smaller than that of the sample inlet channel, so that the sample can flow from the sample-adding area into the reaction chamber under the action of gravity, but cannot flow into the fluid channel.

Due to the pressure balance in the system, the sample in the sample-adding area can realize autonomous transfer to the reaction chamber under the drive of gravity without an external driving device, and this process is easy to realize. Once the sample enters the sample-adding area, it will flow downward by itself and enter the reaction chamber through the sample inlet channel. The main flow channel of the gravity-driven microfluidic chip and the sample inlet channel of the reaction chamber have a large pore size, which can enable the liquid to flow smoothly under the drive of gravity. However, the pore size of the fluid channel is significantly smaller than these flow channels. The fluid channel is a multi-turn tortuous flow channel with a small pore size. Under the action of gravity alone, the liquid cannot enter and flow smoothly. Therefore, after the reaction chamber is filled with the sample, the sample will be blocked when continuing to advance, and it is difficult to flow out from the fluid channel at the top of the reaction chamber. All the samples will remain in the reaction chamber to complete the nucleic acid amplification reaction. After the nucleic acid amplification reaction is completed, the sample can enter the detection chamber through the fluid channel only under the push of pressure, thereby ensuring accurate detection.

In some embodiments, the pore size of the fluid channel does not have to be completely uniform. Different pore sizes can be designed in different parts of the fluid channel to increase the obstruction of the liquid in the fluid channel by means of impact, sharp turns, etc., and increase the difficulty of the sample entering the fluid channel.

In some embodiments, the fluid channel provided by the present invention effectively combines the above several designs, and the design points include the following: 1. The pore size of the fluid channel is smaller than that of the sample inlet channel; 2. The fluid channel is a multi-turn tortuous flow channel with at least one bent structure; 3. The fluid channel includes an arched flow channel, which includes a sample inlet section, a middle section, and a sample outlet section. The middle section is arched upward, the position of the middle section is higher than that of the sample inlet section and the sample outlet section, and at least the position of the middle section is higher than that of the reaction chamber; 4. The position of the detection chamber is higher than that of the reaction chamber, the position of the sample inlet of the detection chamber is higher than that of the sample outlet of the reaction chamber, and the bottom of the detection chamber is not lower than the top of the reaction chamber; any one, two, three, or four of these methods are combined to achieve a better automatic valve effect, thereby accurately realizing the controllable transfer of the sample between the reaction chamber and the detection chamber.

In some embodiments, the pore size of the sample inlet channel is 400 μm-800 μm, and the pore size of the fluid channel is 50-300 μm.

It can be understood that the pore sizes of the sample inlet channel and the fluid channel only need to be able to successfully prepare the gravity-driven microfluidic chip, and the fluid channel can play a certain role in preventing the liquid from easily flowing in due to its small pore size under the action of gravity and surface tension. The limitation on the pore size here does not mean that it cannot be used if it exceeds or is smaller than this pore size, but is only a preferred embodiment. For example, the pore size of the sample inlet channel can be 801 μm, 805 μm, 810 μm, 820 μm, 850 μm, 900 μm, 399 μm, 395 μm, 390 μm, 385 μm, 380 μm, etc., or the pore size of the fluid channel can be 301 μm, 302 μm, 305 μm, 310 μm, 320 μm, 350 μm, 390 μm, 49 μm, 48 μm, 46 μm, 41 μm, 40 μm, 35 μm, etc., which can still be realized under a suitable chip size.

Further, it further includes a main flow channel, the sample inlet channel is in fluid communication with the branch port of the main flow channel, the sample inlet channel is an upwardly arched arc-shaped flow channel, the inlet and outlet of the sample inlet channel are respectively located at the low points on both sides of the arc-shaped flow channel, and the inlet is higher than the outlet; the highest point of the arc-shaped flow channel is not lower than the top of the reaction chamber.

The arc-shaped flow channel design of the sample inlet channel enables the sample to easily enter the sample inlet channel from the branch port and then enter the reaction chamber through the sample inlet channel. However, the sample entering the reaction chamber is difficult to return to the main flow channel from the sample inlet channel. Because the sample inlet of the reaction chamber is located at the lowest point of the sample inlet channel, the sample cannot overcome the gravity and the pressure caused by the upwardly arched arc-shaped flow channel to return to the main flow channel, and can only remain in the reaction chamber, thereby preventing sample backflow in the reaction chamber.

When a specific volume of the sample to be tested is added from the sample-adding area, the cover of the sample-adding area is closed, and the sample flows into the reaction chamber of each detection unit from the main flow channel under the action of gravity. The sample inlet of the reaction chamber is at a low position, which enables the sample to be tested to quickly flow into the reaction chamber and gradually fill it. However, at this time, the sample will not flow out of the reaction chamber from the sample outlet, because the sample outlet is at the top of the reaction chamber, and the fluid channel is a multi-turn tortuous flow channel with a small pore size, so the sample cannot flow into the fluid channel. At the same time, the sample is also difficult to return to the main flow channel from the sample inlet channel, because the sample inlet of the reaction chamber is located at the lowest point of the sample inlet channel, and the highest point of the sample inlet channel is higher than the top of the reaction chamber. The sample cannot return to the main flow channel under the action of gravity. Therefore, all the samples will remain stably in the reaction chamber for reaction.

Further, the exhaust channel is connected to the detection chamber.

The position of the exhaust channel will directly affect the effect of the controllable transfer of the sample between the reaction chamber and the detection chamber. The main reason is that the distance between the exhaust channel and the detection chamber will affect the difficulty of the controllable transfer. If the exhaust channel is too close to the reaction chamber and far from the detection chamber, the difficulty of transferring the sample into the detection chamber will be significantly increased, and even the sample may enter the exhaust channel early and wet the waterproof and breathable membrane, making it unable to breathe again. The inside of the microfluidic chip is completely closed, and the liquid can no longer flow. Therefore, the position of this exhaust channel shared by the reaction chamber and the detection chamber is very critical. It must be ensured that it remains ventilated when the sample enters the reaction chamber, and it is not closed. Even if it is closed during the process, it can quickly resume ventilation.

In some embodiments, for the gravity-driven microfluidic chip, the exhaust channel can be directly arranged in the detection chamber. Therefore, the fluid channel in this chip has a small pore size and is tortuous, which can provide sufficient resistance to enter the detection chamber. Moreover, the exhaust channel is arranged in the detection chamber, which makes it easier to control the flow of the sample and prevents the system from being closed due to the sample entering the exhaust channel. When the sample enters the detection chamber, the sample will enter the exhaust channel to close the system, so that the sample can no longer flow out of the detection chamber, thereby helping to realize the controllable transfer of the sample between the reaction chamber and the detection chamber.

Further, a waterproof and breathable membrane is arranged at the outlet of the exhaust channel of the detection chamber, which is used for controlling the communication state between the detection chamber and the external atmosphere.

The arrangement of the exhaust channel and the waterproof and breathable membrane of the detection chamber can realize the discharge of gas from the reaction chamber and the detection chamber to the outside through the waterproof and breathable membrane, but the liquid cannot be discharged through the waterproof and breathable membrane. When the exhaust channel is filled with liquid, the waterproof and breathable membrane will lose its air permeability, so that the entire system is in a closed state, and the liquid in the system stops flowing. Therefore, the waterproof and breathable membrane can help the sample in the detection chamber establish an internal and external pressure balance, and can regulate the flow direction of the liquid. When the detection chamber is filled with liquid and the waterproof and breathable membrane is wetted through the exhaust channel, the system inside the chip is in a closed state, so that the sample in the detection chamber stops flowing and no backflow occurs. In some embodiments, the waterproof and breathable membrane is made of a polymer material.

Existing nucleic acid multiplex detection microfluidic chips usually need to arrange a valve between the reaction chamber and the detection chamber for blocking. Therefore, both the reaction chamber and the detection chamber need to be provided with exhaust channels and waterproof and breathable membranes respectively.

However, in the gravity-driven microfluidic chip provided by the present invention, the reaction chamber does not need to be provided with an exhaust channel. The multi-turn tortuous fluid channel with a small pore size can act as a valve for temporarily blocking the liquid flow between the reaction chamber and the detection chamber, but the reaction chamber and the detection chamber can always maintain ventilation. Therefore, the reaction chamber and the detection chamber can share the exhaust channel located on the detection chamber for exhaust, and the reaction chamber does not need to be additionally provided with an exhaust channel and a waterproof and breathable membrane. This design makes the structure simpler, the cost lower, the operation more convenient, and at the same time ensures the accuracy of the detection results.

Further, the outlet of the sample inlet channel is located at the bottom of the reaction chamber; the inlet of the fluid channel is located at the top of the reaction chamber; the outlet of the fluid channel is located at the bottom of the detection chamber; the inlet of the exhaust channel is located at the top of the detection chamber.

The outlet of the sample inlet channel is equivalent to the sample inlet of the reaction chamber, and the inlet of the fluid channel is equivalent to the sample outlet of the reaction chamber. The gravity-driven microfluidic chip also designs the positions of the sample inlet and the sample outlet of the reaction chamber. The sample inlet is at the bottom of the reaction chamber, and the sample outlet is at the top of the reaction chamber. The purpose of this design is to ensure that when the sample enters the reaction chamber, it can smoothly exhaust to the sample outlet and maintain pressure balance. At the same time, because the sample outlet is at the top of the reaction chamber, the difficulty of the sample flowing out is increased, and the sample remains more stably in the reaction chamber.

The outlet of the fluid channel is equivalent to the sample inlet of the detection chamber, and the inlet of the exhaust channel is equivalent to the exhaust port of the detection chamber. The sample inlet is at the bottom of the detection chamber, and the exhaust port is at the top of the detection chamber. The purpose of this design is to ensure that when the sample enters the detection chamber, it can smoothly exhaust to the exhaust port and maintain pressure balance. Under the pressure of the pressure-applying area, the sample in the reaction chamber enters the detection chamber from the bottom of the detection chamber through the fluid channel. Since the sample volume is usually sufficient, the sample can also enter the exhaust channel and contact the waterproof and breathable membrane, making the waterproof and breathable membrane lose its air permeability. The inside of the gravity-driven microfluidic chip is in a closed state as a whole, and the sample stops flowing, so that it is completely retained in the detection chamber for the detection reaction and the detection of the target nucleic acid.

Further, it further includes a pressure-applying area, which is located on the chip or on the tube cap used to close the sample-adding area of the chip, and is used for increasing the pressure of the liquid in the reaction chamber so that the liquid in the reaction chamber flows into the detection chamber through the fluid channel.

Further, it further includes a pressure-applying area, which is located on the chip or on the tube cap used to close the sample-adding area of the chip, and is used for increasing the pressure of the liquid in the reaction chamber so that the liquid in the reaction chamber flows into the detection chamber through the fluid channel.

The purpose of setting the pressure-applying area is: after the sample completes the amplification and other reactions in the reaction chamber, the sample needs to completely enter the detection chamber from the reaction chamber, and at this time, it is necessary to overcome a certain air pressure and the pressure caused by the multi-turn tortuous fluid channel with a small pore size. Therefore, the pressure-applying area needs to provide sufficient pressure to drive the sample to be tested to enter the detection chamber from the reaction chamber.

It can be understood that the pressure-applying area can be arranged at any position of the chip, such as the middle of the chip, the bottom of the chip, the side of the chip, the sample-adding area of the chip, the detection area of the chip, etc., and can even be arranged on the tube cap, as long as the airbag of the pressure-applying area is pressed to provide pressure for the liquid in the reaction chamber to smoothly enter the detection area.

In some embodiments, the pressure-applying area is preferably arranged on the upper left or upper right of the chip, such as one side of the sample-adding area of the chip. Because if the pressure-applying area is arranged on the sample-adding area or the tube cap, although it can also provide a certain pressure, the volume of the upper end of the sample-adding area or the tube cap is limited, resulting in very limited air pressure provided, and the pressing operation is inconvenient. When tightening the tube cap, it is also easy to accidentally touch the airbag on the tube cap, causing the airbag to release air in advance. Therefore, the pressure-applying area is preferably arranged on the upper left or upper right of the gravity-driven microfluidic chip, and should also reach a specific volume (the specific volume can be calculated according to the size of the gravity-driven microfluidic chip and the required pressure to set the airbag with a suitable volume), so that the pressing process provides a more accurate air pressure to drive the sample to be tested to enter the detection chamber from the reaction chamber.

The airbag of the pressure-applying area can be pressed manually or by a matching detection device providing a specific force to press the airbag.

In some embodiments, the pressure-applying area can be connected to the sample pool through a shunt channel. By pressing, air is discharged from the shunt channel to the sample pool and into the gravity-driven microfluidic chip.

Further, the gravity-driven microfluidic chip further includes a sample-adding area, and the sample-adding area includes a sample pool and a tube cap. The sample pool is used for containing the sample to be tested, and the tube cap is used for sealing the gravity-driven microfluidic chip.

In some embodiments, the cross-section of the reaction chamber of the gravity-driven microfluidic chip is circular. The circular cross-section can enable the reaction chamber to contain more samples.

In some embodiments, the cross-section of the detection chamber of the gravity-driven microfluidic chip is drop-shaped, and the exhaust port is located at the top of the drop shape.

The drop-shaped cross-section of the detection chamber helps the gas or liquid in the detection chamber to collect upward, making it easier to enter the exhaust channel.

The gravity-driven microfluidic chip provided by the present invention, through the clever design of the structures of the sample inlet channel, fluid channel, chamber, and exhaust channel, does not require additional driving. The sample can enter the reaction chamber by its own gravity without entering the detection chamber, and after completing the amplification reaction in the reaction chamber, it can enter the detection chamber under the action of a simple pressure device, realizing the controllable transfer of the sample in different chambers without sample backflow.

On the other hand, when the chip is a centrifugal microfluidic chip, the exhaust channel is connected to the fluid channel, so that the combination of the exhaust channel and the fluid channel can automatically provide resistance for the sample in the reaction chamber.

The exhaust channel is connected to the fluid channel, which significantly increases the difficulty of the sample entering the detection chamber. In this way, it is difficult for the sample to enter the detection chamber without an external driving force and only through a simple pressure device arranged on the chip. However, this way is especially suitable for centrifugal microfluidic chips. The centrifugal force provided by centrifugation can promote the sample to smoothly enter the detection chamber, and the magnitude of the centrifugal force can be controlled by the centrifugal speed. The centrifugal speed required in this way is not high, and can be completely controlled within the range of medium-speed centrifugation, which will not affect the amplification and detection of the target nucleic acid in the sample.

Further, the chip includes a chip body with a rotation center, the reaction chamber, the detection chamber, the fluid channel, and the exhaust channel are located on the chip body, and the rotation center is used for connecting with a centrifugal driving device to drive the centrifugal microfluidic chip to centrifuge; the exhaust channel includes an inlet and an outlet, the inlet is the connection between the fluid channel and the exhaust channel, and the outlet is the other end of the exhaust channel; the outlet is closer to the rotation center than the inlet, so that the exhaust direction of the exhaust channel is opposite or at least partially opposite to the rotational centrifugal direction.

The centrifugal microfluidic chip provided by the present invention is provided with multiple groups of detection units on the chip body. Each group of detection units is provided with a reaction chamber and a detection chamber, which can be applied to the simultaneous detection of multiple different target analytes, such as the detection of multiple different nucleic acid targets, and each group of detection units can realize nucleic acid multiplex detection. Of course, it can be understood that the centrifugal microfluidic chip of the present application is not only used for nucleic acid multiplex detection, but also for the detection of other molecules such as proteins and antibodies. The reaction chamber can be used for nucleic acid amplification of the sample, and the detection chamber can be used for sample detection.

During the use of the centrifugal microfluidic chip, it needs to be kept in a horizontal state, with the sample inlet facing upward. In some embodiments, the centrifugal microfluidic chip can be inserted into a matching detection device, so that the centrifugal microfluidic chip is placed horizontally with the sample inlet facing upward, and the rotation center is buckled and fixed with the rotating shaft for centrifugation of the detection device, so that the rotating shaft can drive the centrifugal microfluidic chip to perform centrifugal rotation; in some embodiments, the detection device can provide a heat source to heat the reaction chamber of the centrifugal microfluidic chip; in some embodiments, the detection device can also detect the fluorescent substances generated after the sample reaction in the centrifugal microfluidic chip and read the detection results.

Since the centrifugal microfluidic chip needs to control the flow of liquid through centrifugation, it is not possible to provide an exhaust channel for each reaction chamber and detection chamber. Because if each chamber is provided with an exhaust channel, it is difficult to control the flow of the sample during centrifugation, and the sample may directly pass through the reaction chamber to reach the detection chamber under the action of centrifugation, affecting the amplification and detection results. Therefore, existing centrifugal microfluidic chips usually only set air vents on the chip floor or cover plate. This setting is not suitable for nucleic acid multiplex detection chips, because it is necessary to set multi-stage chambers. The subsequent chambers are difficult to exhaust during centrifugation, and a larger centrifugal force is required to push the sample to enter. High-speed centrifugation easily causes the pre-embedded freeze-dried reagents to dissolve insufficiently and precipitate, thereby seriously affecting the accuracy of the detection results.

The centrifugal microfluidic chip provided by the present invention is provided with an exhaust channel on the fluid channel connecting the reaction chamber and the detection chamber in each detection unit, so that the reaction chamber and the detection chamber share one exhaust channel. By improving the structure of the exhaust channel and combining the performance of centrifugation, the reaction chamber and the detection chamber can smoothly exhaust when exhaust is needed, and smoothly close to prevent interference when reaction is needed. It can also effectively control the amount of sample entering the reaction chamber and the detection chamber. Nucleic acid multiplex detection can be completed with only one or two centrifuging steps, and the problems of liquid leakage or sample backflow can be perfectly avoided. After the sample to be tested enters the corresponding chamber, it can remain stably in the chamber, no backflow occurs, and it will not flow to other chambers, thus ensuring that each step of the reaction can be completed smoothly and providing a guarantee for the accurate detection of the centrifugal microfluidic chip.

The fluid channel connecting the reaction chamber and the detection chamber refers to the channel connecting the outlet of the reaction chamber and the inlet of the detection chamber.

Further, the exhaust channel includes an inlet and an outlet, the inlet is the connection between the fluid channel and the exhaust channel, and the outlet is the other end of the exhaust channel; the outlet is closer to the rotation center than the inlet, so that the exhaust direction of the exhaust channel is opposite or at least partially opposite to the rotational centrifugal direction.

When the centrifugal microfluidic chip is centrifuged, the flow direction of the fluid sample under the action of centrifugation is from the center of the circle to the outside. In the centrifugal microfluidic chip provided by the present invention, the exhaust channel is led out from the fluid channel between the reaction chamber and the detection chamber and then approaches the rotation center, so that the exhaust direction is opposite to the centrifugal direction. The fluid sample will not enter the exhaust channel during centrifugation, but accurately enter the corresponding chamber along the centrifugal direction.

In some embodiments, the exhaust channel is led out from the middle of the fluid channel or a position close to the detection chamber, and the initial section of the lead-out is an arc-shaped channel, and the angle between the arc-shaped channel and the fluid channel close to the reaction chamber is less than 90 degrees.

In some embodiments, the exhaust channel can be led out slightly close to the detection chamber. Because if the exhaust channel is led out close to the detection chamber, the pressure that the sample needs to overcome to enter the detection chamber during centrifugation can be reduced, and the sample can more easily enter the detection chamber during centrifugation.

When the exhaust channel is led out from the fluid channel, the smaller the angle between the initial section and the fluid channel close to the reaction chamber, the more difficult it is for the fluid to enter the exhaust channel. However, this angle cannot be too small, otherwise it may cause difficulty in exhaust. It is preferable to use an arc-shaped channel at one end for transition, and the angle between the arc-shaped channel and the fluid channel close to the reaction chamber is less than 90 degrees.

In some embodiments, the inner diameter of the exhaust channel is smaller than that of the fluid channel.

Further, the outlet of the exhaust channel has two setting modes: one is connected to the outside, and a waterproof and breathable membrane is arranged at the outlet connected to the outside; the other is in communication with the main flow channel of the chip body.

Further, the outlet of the exhaust channel has two setting modes: one is connected to the outside, and a waterproof and breathable membrane is arranged at the outlet connected to the outside; the other is in communication with the main flow channel of the chip body.

When the first setting mode is adopted, nucleic acid multiplex detection can be completed with only one centrifugation in the whole process; when the second setting mode is adopted, nucleic acid multiplex detection can be completed with only two centrifuging steps in the whole process.

The first setting mode is to exhaust to the outside. The exhaust port is covered with a waterproof and breathable membrane. The waterproof and breathable membrane is a membrane that can allow air to pass through but not water, which can realize the discharge of gas from the reaction chamber or the detection chamber, but the liquid cannot pass through. The waterproof and breathable membrane can help the sample in the reaction chamber or the detection chamber establish an internal and external pressure balance, and can regulate the flow direction of the liquid, so that the sample in the reaction chamber or the detection chamber will not leak or flow back. In some embodiments, the waterproof and breathable membrane is made of a polymer material.

The flow process of the sample to be tested in the centrifugal microfluidic chip mainly includes two steps: the first step, the sample to be tested enters the reaction chamber through the main flow channel under the action of injection pressure for reaction; the second step, after the reaction is completed, the sample to be tested flows out of the reaction chamber through low-speed centrifugation and enters the detection chamber for reaction and detection.

When the first setting mode is adopted, the sample enters the main flow channel from the sample-adding port through a pipette or syringe injection, and flows into each reaction chamber from the main flow channel under the action of injection pressure, then the sample-adding port is sealed. In the first step, during the process of the sample entering the reaction chamber, the gas in the reaction chamber needs to be discharged to the outside through the exhaust channel. Since the detection chamber is not provided with an exhaust channel, the sample is difficult to flow into it. At the same time, because the exhaust direction of the exhaust channel is opposite to the centrifugal direction and higher than the fluid channel, and the inner diameter of the exhaust channel is smaller than that of the fluid channel, the sample can only reach the intersection of the fluid channel and the exhaust channel at most in the first step, and cannot continue to move forward, thus controlling the volume of the sample entering the reaction chamber. At this time, since the sample-adding port is sealed, that is, only the exhaust channel of the entire microfluidic chip is connected to the atmosphere, and the other parts are in a sealed state, the internal and external pressure balance is achieved. The sample in the reaction chamber can no longer flow from the reaction chamber to other areas, because flowing out of the reaction chamber will inevitably require a large pressure to break the established internal and external pressure balance. Therefore, all the samples will remain in the reaction chamber for reaction, and no leakage or backflow will occur.

In the second step, after the sample completes the reaction in the reaction chamber, through low-speed centrifugation (such as 100-1000 rpm), the initial pressure balance is broken, and the sample flows out of the reaction chamber under the action of centrifugal force. At this time, because the exhaust direction of the exhaust channel is opposite to the centrifugal direction, the sample will not enter the exhaust channel, but will all flow to the detection chamber, and the gas in the detection chamber is discharged from the exhaust channel. At this time, the pressure balance is also achieved in the detection chamber, so that the sample in the detection chamber can no longer flow out of the detection chamber, and all remain in the detection chamber for reaction and detection.

Therefore, when the first setting mode is adopted, under the combined action of the exhaust channel and the fluid channel, the sample in the reaction chamber or the detection chamber can establish an internal and external pressure balance, the flow direction of the liquid can be regulated, the sample in the reaction chamber or the detection chamber will not leak or flow back, and the amount of sample entering the reaction chamber or the detection chamber can be effectively controlled. Nucleic acid multiplex detection can be completed with only one low-speed centrifugation.

The second setting mode is to exhaust to the main flow channel. Neither the reaction chamber nor the detection chamber needs to be connected to the external atmosphere. The exhaust channel is directly connected to the main flow channel, and the atmosphere pressed out of the reaction chamber or the detection chamber is discharged back to the main flow channel, which is equivalent to discharging the air pressure in the liquid-filled part to the unfilled part through the exhaust channel, playing a role in controlling the flow of liquid. Moreover, after exhausting to the main flow channel through the exhaust channel, the pressure balance is achieved inside the chip, and the sample entering the reaction chamber or the detection chamber is also difficult to flow out of the reaction chamber, and backflow is difficult to occur, forming a circular closed environment. Compared with the first setting mode, since it is not connected to the outside, aerosol contamination can be effectively avoided, and the detection result is more accurate and reliable. At the same time, since the second setting mode omits the steps of preparing air vents and covering waterproof and breathable membranes, the preparation process is simpler and easier, and the cost is lower.

This process includes three steps: the first step, the sample enters the main flow channel from the sample-adding port; the second step, low-speed centrifugation is performed to make the sample enter the reaction chamber from the main flow channel for reaction; the third step, after the reaction is completed, the sample flows out of the reaction chamber through medium-speed centrifugation and enters the detection chamber for reaction and detection. Different from the first setting mode, when the second setting mode is adopted, it is also necessary to control the flow of the sample by controlling the centrifugal speed. The sample enters the reaction chamber from the main flow channel through low-speed centrifugation (such as 100-1000 rpm), and then enters the detection chamber from the reaction chamber through medium-speed centrifugation (such as 1000-3000 rpm). The method of controlling the flow of the sample in different chambers by controlling the centrifugal speed is simpler and easier to operate, and the whole process is more controllable, and the detection result is more accurate.

In the first step, the sample enters the main flow channel from the sample-adding port through a pipette or syringe injection. After filling the serpentine main flow channel, the sample will flow to the final waste liquid pool, then the injection is stopped, and the sample-adding port is sealed. This process is equivalent to the process of filling the main flow channel with the sample, and also the process of discharging the gas after being filled with the liquid to the unfilled part of the main flow channel through the exhaust channel. Since the inner diameter of the branch channel connecting the main flow channel and the reaction chamber is small, and there is a large difference between the inner diameter of the main flow channel and the branch channel, the sample cannot flow into the reaction chamber. Moreover, it is difficult to discharge the gas in the reaction chamber back to the main flow channel from the exhaust channel only by the injection pressure, so the sample will stay in the main flow channel first.

In the second step, after the main flow channel is completely filled with the sample, clockwise low-speed instantaneous centrifugation is performed. The sample in the main flow channel enters the reaction chamber under the action of centrifugal force. Since the detection chamber is not provided with a separate exhaust channel, it is almost in an enclosed structure. Under low-speed centrifugation, the sample is difficult to overcome the gas pressure in the detection chamber, so it cannot flow into the detection chamber. At the same time, because the exhaust direction of the exhaust channel is opposite to the centrifugal direction and the exhaust channel is higher than the fluid, the sample can only reach the intersection of the fluid channel and the exhaust channel at most in the second step, and cannot continue to move forward, thus controlling the volume of the sample entering the reaction chamber. During the low-speed centrifugation, the sample enters the reaction chamber, and at the same time, the gas in the reaction chamber is discharged back to the main flow channel, which can also help the sample in the main flow channel flow into the reaction chamber. After low-speed centrifugation, the sample remains in the reaction chamber for amplification reaction. In a sealed state, the internal and external pressure balance is achieved. The sample in the reaction chamber can no longer flow from the reaction chamber to other areas, because flowing out of the reaction chamber will inevitably require a large pressure to break the established internal and external pressure balance. Therefore, all the samples will remain in the reaction chamber for reaction, and no leakage or backflow will occur.

In the third step, after the sample completes the amplification reaction in the reaction chamber, the centrifugal speed is increased, and medium-speed centrifugation is performed. The initial pressure balance is broken, and the sample is driven to transfer from the reaction chamber to the detection chamber. At this time, because the exhaust direction of the exhaust channel is opposite to the centrifugal direction and the inner diameter of the exhaust channel is smaller, the sample will not enter the exhaust channel, but will all flow to the detection chamber. Since the entire system is in a sealed state, the liquid in the detection chamber will not flow back, thus being stably used for detection.

Therefore, the second setting mode adopts two-stage centrifugation to complete nucleic acid multiplex detection. The first-stage centrifugation speed and the second-stage centrifugation speed can be quite different, and the control is stable. Moreover, high-speed centrifugation (such as 3000-10000 rpm) is not required, which avoids the precipitation of pre-embedded freeze-dried or vitrified reagents due to insufficient dissolution caused by high-speed centrifugation, thereby causing reaction failure and affecting the detection results.

Further, when the centrifugal microfluidic chip is placed horizontally, the outlet of the exhaust channel is higher than the inlet; when the outlet of the exhaust channel is connected to the outside, a gas-permeable chamber is further arranged at the outlet of the exhaust channel, and the volume of the gas-permeable chamber is not less than that of the reaction chamber.

The outlet of the exhaust channel is higher than the inlet, which can further increase the difficulty of the fluid sample entering the exhaust channel, thereby helping to control the flow of liquid in the chip, and at the same time facilitating the discharge of gas from the reaction chamber and the detection chamber.

The arrangement of the gas-permeable chamber can help the reaction chamber and the detection chamber to exhaust, making it easier for the sample to enter the reaction chamber, and also facilitating the transfer of gas from the detection chamber to the gas-permeable chamber during centrifugation.

Further, the chip body is circular; when the outlet of the exhaust channel is connected to the outside, the main flow channel is arranged in a wave shape; when the outlet of the exhaust channel is in communication with the main flow channel, the main flow channel is arranged in a serpentine shape; the main flow channel is bent into a circular ring, and the chip body, the circular ring, and the rotation center are coaxially arranged.

In some embodiments, the main flow channel adopts a serpentine (wave-shaped) flow channel with a large curvature and uniformly spaced arrangement, which can help maintain the pressure balance inside the chip, stably control the flow of liquid, and help prevent direct mutual interference between each detection unit.

In some embodiments, when the outlet of the exhaust channel is in communication with the main flow channel, the main flow channel is arranged in a serpentine shape. The serpentine arrangement makes the main flow channel have a larger bending degree and a longer length, so that the sample can stay stably in the main flow channel first.

Further, the serpentine-arranged main flow channel includes U-shaped flow channels arranged at equal distances along the circular ring, and the bottom end of the U-shaped flow channel is connected to the reaction chamber; when the outlet of the exhaust channel is in communication with the main flow channel, the communication part is the top end of the U-shaped flow channel.

In some embodiments, the bottom end of the U-shaped flow channel is provided with a branch channel connected to the inlet of the reaction chamber, the top end of the U-shaped flow channel is connected to the exhaust channel, the left and right sides of the U-shaped flow channel are longer, and the top end is closer to the rotation center than the bottom end. As a result, the distance between the bottom end and the top end of the U-shaped flow channel is relatively far. When the exhaust channel exhausts to the top end of the U-shaped flow channel, it will not immediately affect the pressure balance at the bottom end, which helps the sample flow into the reaction chamber smoothly.

The U-shaped flow channels are arranged uniformly, and the bottom end of each U-shaped flow channel is connected to a reaction chamber. Therefore, during centrifugation, the sample in each U-shaped flow channel will enter the corresponding reaction chamber under the action of centrifugal force, which is equivalent to the U-shaped flow channel pre-distributing the sample uniformly, and can assist in controlling the volume of the sample entering the reaction chamber.

Further, the main flow channel is further provided with a sample-adding port; when the outlet of the exhaust channel is in communication with the main flow channel, the sample-adding port is located at the starting end of the main flow channel, and a waste liquid pool is further arranged at the end of the main flow channel.

When the first setting mode is adopted for the exhaust channel, the main flow channel is a connected circular closed loop, and only one opening needs to be set in the main flow channel for sample injection.

When the second setting mode is adopted for the exhaust channel, that is, the sample-adding port is located at the starting end of the main flow channel, the two ends of the main flow channel are not connected, and a sample-adding port and a waste liquid pool are respectively set at the two ends. The sample-adding port is located at the starting end of the main flow channel, and the waste liquid pool is located at the end of the main flow channel.

The waste liquid pool has a liquid storage function and is used for receiving excess samples. During the process of filling the main flow channel with the sample, the excess sample will enter the waste liquid pool. When the second mode is adopted, since the injection pressure is not sufficient to make the sample enter the reaction chamber, after the main flow channel is filled with the sample, the excess sample will start to enter the waste liquid pool. At this time, it also indicates that the sample volume is sufficient, and the sample addition can be stopped. Therefore, the waste liquid pool also has the effect of helping to quantify the sample.

In some embodiments, the surface of the sample-adding port of the centrifugal microfluidic chip is covered with a transparent film for sealing. During sample addition, a syringe is used to pierce the transparent film for sample addition. After the main flow channel is filled with the sample, the surface of the sample-adding port needs to be covered with a transparent film again for sealing.

Further, it further includes one or more sub-chips, the sub-chips are concentric circular ring chips located outside the chip body, and the sub-chips are also provided with a main flow channel, a reaction chamber, and a detection chamber; when the chip body rotates centrifugally, the sub-chips can rotate centrifugally along with the chip body.

The centrifugal microfluidic chip provided by the present invention decomposes the traditional single centrifugal microfluidic chip into a chip body and sub-chips. The chip body and sub-chips operate independently, and have the advantages of small size and convenient operation. They can perform multi-indicator detection of multiple samples at the same time, greatly improving the detection efficiency, meeting the needs of high-throughput diagnosis with high speed, convenience, and low cost, and have important application value in the fields of clinical diagnosis or rapid on-site screening.

The chip body and the sub-chips can be directly molded and manufactured in one chip, directly connected into an integrated structure, or manufactured separately and fixed by means of buckles. In some embodiments, in order to make the centrifugation process more stable and controllable, the chip body and the sub-chips adopt an integrated structure and cannot be disassembled.

In some embodiments, the sub-chips, the chip body, and the entire microfluidic chip are all in the same plane. The sub-chips are located outside the chip body and have a larger diameter, so more groups of detection units can be set. At the same time, the sub-chips can also be provided with sample-adding ports and waste liquid pools to independently perform nucleic acid multiplex detection, and can detect samples different from those of the chip body, realizing the simultaneous detection of multiple samples. In addition, more indicators can be detected at the same time (each detection unit can detect one nucleic acid target), significantly increasing the detection throughput and improving the detection efficiency.

In some embodiments, the number of sub-chips and the number of detection units in each sub-chip or chip body can be flexibly set according to the number of samples to be detected and the requirements of indicator detection. The solid reagents pre-embedded in the reaction chamber and the detection chamber of each detection unit can be flexibly set according to actual needs. For example, different amplification primers for respiratory targets are pre-embedded in different reaction chambers, and corresponding detection reagents are pre-embedded in the corresponding detection chambers, so that multiplex detection of respiratory pathogens can be flexibly realized.

In some embodiments, the present invention also provides a series of centrifugal microfluidic chip and its variants, with the second type of exhaust channel setting (exhaust channel in communication with the main flow channel) and the core centrifugal driving logic, and optimize the spatial layout to realize multi-sample parallel detection while retaining all technical advantages of the aforementioned centrifugal microfluidic chip.

This microfluidic chip and its variants include but are not limited three structural forms: integral full-disk chip, two-split disk chip, and four-split disk chip. All variants adopt an integrated single-layer circular chip body, with a rotation center designed as a clamping groove to ensure stable fixation and coaxial rotation with the centrifugal driving device.

The integral full-disk chip is a monolithic circular structure without sub-chips, with multiple groups of detection units radially arranged along the circular ring of the chip body (7-30 groups optional).

In some embodiments, each group of detection units includes a reaction chamber (for nucleic acid amplification) and a detection chamber (for sample detection), connected through a first channel as in liquid communication between the reaction chamber and the detection chamber.

In some embodiments, a second channel for receiving the liquid sample has or did not have target nucleic acid therein. The second channel is liquid communication with the reaction chamber by a third channel which is directly connected with the reaction chamber, an exhaust channel with first end and second end, the first end is gas or air communication with the reaction chamber or/and reaction chamber, the second end of the exhaust channel is connected with the second channel. When the liquid sample is injected into to the second channel, firstly, the liquid sample will fill into the second channel or entering into the reaction chamber. In this situation, the exhausted channel can exhaust the air in the reaction chamber to the second channel as to exhausted air from the reaction chamber into second channel that is communication with external atmospheric out of the chip. In some embodiments, the chip is centrifuged chip that can be derived by a centrifugal device as to connect the rotation center that located in the chip.

In some embodiments, the first end of the exhaust channel is opposite to the rotational centrifugal direction, and the exhaust channel is in communication with the circular second liquid channel. The chip is equipped with a single sample-adding port at the starting end of the second liquid channel and a waste liquid pool at the tail end, realizing the detection of one sample per chip. If many detection unites are included in the chip and each of detection unites is liquid communication with the second channel by means of parallel connection. Each unite detection one of target nucleic acids, if many detection unite, many types of the target nucleic acids can be detected as the same time. It is understood that one sample is inject into the second channel and the sample will be spited to each detection unites and many types of the target nucleic acids can be detected as the same chip and at the same time.

The flow channel unit retains the core design of the second, first, third liquid channel, air exhaust channel, reaction chamber, and detection chamber, ensuring that the sample can be controllably transferred between the reaction chamber and the detection chamber through low-speed and medium-speed two-stage centrifugation, avoiding sample backflow and aerosol contamination.

The two-split disk chip divides the circular chip body into two symmetric fan-shaped independent functional regions along the diameter. Each functional region is independently equipped with a serpentine main flow channel (second channel), radial detection units (15 groups optional per region), a sample-adding port, and a waste liquid pool. The two functional regions are fluidically isolated, supporting simultaneous detection of two different samples or two batches of the same sample. Each functional region retains the same channel and chamber design as the integral full-disk chip, including the U-shaped flow channel distribution, exhaust channel-main flow channel communication mode, and two-stage centrifugation control logic, ensuring consistent detection accuracy and operational convenience.

The four-split disk chip divides the circular chip body into four equal fan-shaped independent functional regions. Each functional region is a miniaturized independent detection unit set, including a shortened serpentine main flow channel, radial detection units (7 groups optional per region), a sample-adding port, and a waste liquid pool. The four functional regions are mutually fluidically isolated, suitable for simultaneous detection of four different samples or four different indicators of a single sample. The cross-sectional dimensions of the main flow channel, fluid channel, and exhaust channel are consistent with the integral full-disk chip, ensuring stable fluid control and sample volume accuracy.

All split-disk variants retain the core technical advantages of the aforementioned centrifugal microfluidic chip: nucleic acid multiplex detection is completed through two-stage centrifugation (low-speed 100-1000 rpm for sample entering the reaction chamber, medium-speed 1000-3000 rpm for sample transferring to the detection chamber), avoiding high-speed centrifugation-induced precipitation of pre-embedded freeze-dried reagents; the closed system design (exhaust channel communicating with the main flow channel) effectively prevents aerosol contamination; the integrated single-layer structure is compact and lightweight, reducing manufacturing difficulty and cost. The split-disk design significantly improves the flexibility of sample detection: the integral full-disk chip is suitable for single-sample multi-indicator detection, the two-split disk chip for small-batch dual-sample parallel detection, and the four-split disk chip for multi-sample rapid screening, which can meet the diverse needs of point-of-care testing (POCT) such as clinical batch detection and on-site rapid screening.

The above split-disk centrifugal microfluidic chips, together with the aforementioned centrifugal microfluidic chip, form a complete product series, all based on the design concept of “shared exhaust channel+two-stage centrifugation+closed system”, realizing the organic unity of high throughput, high accuracy, and operational simplicity, and having important application value in the fields of multi-sample multi-indicator nucleic acid detection such as respiratory pathogens and infectious diseases.

Further, the rotation center is a clamping groove, which can clamp the centrifugal microfluidic chip on the rotating shaft of the centrifugal driving device for centrifugal rotation.

In some embodiments, the clamping groove is a circular clamping groove with protrusions, and the shape of the protrusions is triangular, trapezoidal, etc. The rotating shaft on the detection device is also provided with a fixing groove matching the size of the clamping groove, so that the centrifugal microfluidic chip is fixed on the rotating shaft through the clamping groove and rotates centrifugally along with the rotating shaft.

In some embodiments, the cross-section of the reaction chamber is circular.

The cross-section of the reaction chamber of the centrifugal microfluidic chip is circular, so that the sample to be tested can react in the reaction chamber and be discharged without leaving dead corners, and it is not easy to remain during the inflow and outflow processes.

The cross-section of the detection chamber of the centrifugal microfluidic chip is approximately semicircular and larger than that of the reaction chamber, which can ensure that all samples in the reaction chamber enter the detection chamber.

It can be understood that whether it is a gravity-driven microfluidic chip or a centrifugal microfluidic chip, the solid reagents pre-embedded in the reaction chamber and the detection chamber can be flexibly set. Different reagents are pre-embedded in different chambers. For example, amplification primers for targets are pre-embedded in the reaction chamber, and detection probes for different targets are pre-embedded in the detection chamber.

On the other hand, the present invention provides a nucleic acid detection method, which is characterized in that the nucleic acid multiplex detection microfluidic chip as described above is used for detection. When the nucleic acid multiplex detection microfluidic chip is a centrifugal microfluidic chip, the detection method is divided into two types:
A. When the outlet of the exhaust channel is connected to the outside, the detection method includes the following steps:
(1) Place the centrifugal microfluidic chip horizontally, and add the sample to be tested from the sample-adding port;
(2) The sample to be tested enters the reaction chamber of the centrifugal microfluidic chip from the sample-adding area, and undergoes temperature incubation for nucleic acid amplification;
(3) Clamp the clamping groove of the centrifugal microfluidic chip on the rotating shaft of the detection device;
(4) Centrifuge to make the sample to be tested after nucleic acid amplification enter the detection chamber for reaction;
(5) Read the detection result;
B. When the outlet of the exhaust channel is connected to the main flow channel, the detection method includes the following steps:
(a) Place the centrifugal microfluidic chip horizontally, and add the sample to be tested from the sample-adding port;
(b) Clamp the clamping groove of the centrifugal microfluidic chip on the rotating shaft of the detection device;
(c) Perform low-speed centrifugation to make the sample to be tested enter the reaction chamber of the centrifugal microfluidic chip from the sample-adding area for nucleic acid amplification;
(d) Perform medium-speed centrifugation to make the sample to be tested after nucleic acid amplification enter the detection chamber for reaction;
(e) Read the detection result;
When the nucleic acid multiplex detection microfluidic chip is a gravity-driven microfluidic chip, the detection method includes the following steps:
(I) Vertically insert the microfluidic chip into the detection device;
(II) The sample enters the microfluidic chip from the sample-adding port, flows into the reaction chamber under the action of gravity, and cover the tube cap;
(III) The sample to be tested undergoes nucleic acid amplification in the reaction chamber;
(IV) Squeeze the pressure-applying area to make the sample to be tested after nucleic acid amplification enter the detection chamber for reaction;
(V) Read the detection result

In some embodiments, in step (2), (c) or (III), the nucleic acid amplification is isothermal amplification, and a heating device is required to provide the reaction chamber with a suitable temperature for isothermal amplification (such as 30-65 °C).

On the other hand, the present invention provides a microfluidic chip. The microfluidic chip is a nucleic acid typing detection chip, which is different from the aforementioned nucleic acid multiplex detection microfluidic chip. It is only provided with reaction chambers and no detection chambers, and is more suitable for nucleic acid typing detection, and can be used for detecting different target nucleic acids in pre-processed samples. However, in some embodiments, the nucleic acid typing detection chip is suitable for both genotyping detection and nucleic acid multiplex detection. When used for nucleic acid multiplex detection, the liquid storage chamber is used for lysis of target nucleic acids, and the reaction chambers need to be pre-embedded with amplification and detection reagents for different nucleic acids; when used for nucleic acid typing detection, the liquid storage chamber is used for amplification of target nucleic acids, the liquid storage chamber needs to be pre-embedded with nucleic acid amplification reagents, and the reaction chambers need to be pre-embedded with detection reagents for different target nucleic acids.

The nucleic acid typing detection chip includes a liquid storage chamber for adding or storing reagents and a reaction chamber for amplification or detection; a sample inlet channel is arranged between the liquid storage chamber and the reaction chamber, which facilitates the flow of liquid from the liquid storage chamber to the reaction chamber under the action of gravity; the reaction chamber is further provided with an exhaust channel, and the exhaust channel is finally connected to the liquid storage chamber to discharge the gas between them back to the liquid storage chamber.

Existing microfluidic chips generally have certain drawbacks. For example, the chip structure is complex, flow channel blockage is prone to occur, and they rely on power devices and centrifugal equipment as driving forces to realize the directional movement of liquid, thus having the problem of relying on electricity and centrifugal equipment. At the same time, existing microfluidic chips usually need to be provided with exhaust holes connected to the outside to realize the normal flow of liquid in the chip, which makes the structure of the chip complex and requires preventing liquid leakage.

The nucleic acid typing detection chip provided by the present invention is provided with a liquid storage chamber and a plurality of reaction chambers from top to bottom, and the chambers are connected through flow channels. When there is a sample in the liquid storage chamber, the sample can enter the main flow channel under the action of gravity, and the end of the main flow channel is radially connected to a plurality of reaction chambers; the reaction chamber is provided with a sample inlet channel and an exhaust channel. The sample inlet channel is designed to have a large pore size, which facilitates the flow of liquid from the liquid storage chamber to the reaction chamber under the action of gravity. The exhaust channel is a multi-turn tortuous flow channel with a small pore size, and is finally connected to the liquid storage chamber. When the sample enters the reaction chamber, the gas in the chamber can flow back to the liquid storage chamber through the exhaust channel without gas exchange with the outside, avoiding aerosol contamination. Due to the pressure balance in the system, the sample entering the reaction chamber is also difficult to flow out of the reaction chamber, and backflow is difficult to occur, forming a circular closed environment. Therefore, the sample in the liquid storage chamber can realize autonomous transfer to the reaction chamber under the drive of gravity without an external driving device. The chip greatly simplifies the structure and operation process on the basis of ensuring detection precision, has a fast reaction speed, and avoids aerosol contamination due to closed reaction. It can be extended to the rapid and visual detection of other pathogen nucleic acids in respiratory tract, reproductive tract samples, etc., and is suitable for large-scale promotion and application.

During the use of the nucleic acid typing detection chip, it needs to be kept in a vertical state, with the sample inlet facing upward. In some embodiments, the nucleic acid typing detection chip can be inserted into a matching detection device, so that the microfluidic chip is placed vertically with the sample inlet facing upward, and the detection device can provide a heat source to heat specific positions of the nucleic acid typing detection chip (such as the reaction chamber, valve block, etc.); in some embodiments, the detection device can also detect the fluorescent substances generated after the sample reaction in the nucleic acid typing detection chip and read the detection results.

Further, the reaction chamber is further provided with a sample inlet channel, and the cross-sectional area of the sample inlet channel is larger than that of the exhaust channel, so that the sample can flow into the reaction chamber from the sample inlet channel but cannot flow out of the reaction chamber from the exhaust channel.

Further, the pore size of the sample inlet channel is larger than that of the exhaust channel.

In some embodiments, the pore size of the sample inlet channel is larger than 400 μm.

In some embodiments, the pore size of the sample inlet channel is 400-800 μm.

The main flow channel of the nucleic acid typing detection chip and the sample inlet channel of the reaction chamber, which are used for sample flow, have pore sizes set to enable the liquid to flow smoothly. However, the pore size of the exhaust channel of the reaction chamber is significantly smaller than these flow channels. The exhaust channel is a multi-turn tortuous flow channel with a small pore size, which can only allow gas to pass through and is not smooth for liquid discharge. Therefore, after the reaction chamber is filled with the sample, the sample will be blocked when continuing to advance, and it is difficult to flow out from the exhaust channel of the reaction chamber, and will remain in the reaction chamber to complete the reaction and achieve accurate detection.

Further, the pore size of the exhaust channel is smaller than 300 μm.

In some embodiments, the pore size of the exhaust channel is 50-300 μm.

Further, the exhaust channel is a multi-turn tortuous flow channel, which is provided with at least one bent structure.

The arrangement of a plurality of bent structures in the exhaust channel can extend the length of the exhaust channel as much as possible, so that there are more pipes to store the discharged gas, and prevent the situation that the pressure increases due to the large number of reaction chambers and only one liquid storage chamber, which makes it difficult to fully accommodate all the gas.

In some embodiments, the exhaust channel is further provided with a small chamber at the bent part for accommodating the excess discharged gas.

In some embodiments, the exhaust channel is provided with at least one back-and-forth bent channel.

In some embodiments, before the exhaust channel is connected to the liquid storage chamber, an exhaust chamber can also be set for storing the excess discharged gas, and after the exhaust chamber is filled, the gas continues to return to the liquid storage chamber.

Further, the number of the reaction chambers is at least one, and each reaction chamber is in fluid communication with the main flow channel of the microfluidic chip through a sample inlet channel.

The nucleic acid typing detection chip is provided with a plurality of reaction chambers, which is suitable for multiplex detection of different target nucleic acids or detection of different genotypes. Of course, it can be understood that the nucleic acid typing detection chip is not only used for genotyping detection or nucleic acid multiplex detection, but also for the detection of other molecules such as proteins and antibodies. The liquid storage chamber can be used for sample pretreatment, and the reaction chamber can be used for sample detection.

In some embodiments, the number of the reaction chambers is 2-10. The reaction chambers can be arranged in any way, such as horizontal arrangement, vertical arrangement, or radial arrangement at the end of the main channel in any other way. A plurality of branch ports can also be set at the lower end of the main channel for the arrangement of the reaction chambers.

Further, the reaction chamber is provided with a sample inlet and an exhaust port, the sample inlet is in fluid communication with the sample inlet channel, and the exhaust port is in gas communication with the exhaust channel; the position of the exhaust port is higher than that of the sample inlet.

Further, the sample inlet is located at the lower part of the reaction chamber, and the exhaust port is located at the upper part of the reaction chamber.

The present invention also designs the positions of the sample inlet and the exhaust port of the reaction chamber. The sample inlet is at the lower part of the reaction chamber, and the exhaust port is at the upper part of the reaction chamber. The purpose of this design is to ensure that after the sample enters the reaction chamber, it can smoothly exhaust upward, and at the same time, it can stay in the reaction chamber more stably to prevent backflow.

After the sample to be tested completes the reaction conducive to detection (such as reaction with detection reagents for signal amplification, etc.) in the reaction chamber, the result detection is directly carried out in the reaction chamber. One side of the reaction chamber is made of a transparent film, and the detection instrument can detect the fluorescent substances generated by the reaction through the film and read the detection result.

Further, the liquid storage chamber is provided with a liquid outlet and a gas inlet, the liquid outlet is in fluid communication with the main flow channel, and the gas inlet is in gas communication with the exhaust channel of the reaction chamber; the position of the gas inlet is higher than that of the liquid outlet.

The position of the gas inlet in the liquid storage chamber is higher than that of the liquid outlet, which can ensure that all samples in the liquid storage chamber are discharged smoothly and prevent interference from the incoming gas.

Further, a valve block can be added between the liquid storage chamber and the reaction chamber to control the fluid communication state between the liquid storage chamber and the reaction chamber; the valve block has two states: solid and liquid; when the valve block is solid, the liquid storage chamber and the reaction chamber cannot be in fluid communication; when the valve block is liquid, the liquid storage chamber and the reaction chamber are in fluid communication.

Further, when the valve block is solid, it is located in the main flow channel connecting the liquid storage chamber and the reaction chamber to prevent the sample to be tested in the liquid storage chamber from flowing into the reaction chamber; when the valve block is liquid, it will flow to the exhaust channel of the reaction chamber; the valve block is paraffin or lipid substance, which is solid at room temperature and melts into liquid after heating; the density of the valve block is less than that of water.

Since the microfluidic chip is provided with a plurality of reaction chambers, and the sample inlets of the reaction chambers are gathered together, sample backflow is prone to occur, causing the sample to overflow from one reaction chamber and enter another reaction chamber, resulting in cross-influence. Therefore, how to prevent sample backflow and ensure the accuracy of detection results is very important.

The nucleic acid typing detection chip is provided with an exhaust channel in the reaction chamber, and a valve block is arranged between the liquid storage chamber and the reaction chamber. By using the temperature-induced phase change property of the valve block, the blocking and communication between the liquid storage chamber and the reaction chamber are realized. After the valve block is heated and melted, the flow channels between the liquid storage chamber and each reaction chamber are connected, and the sample in the liquid storage chamber flows into each reaction chamber. The melted valve block has a low density and will float on the top of the sample, flow into the reaction chamber along with the sample, and condense near the exhaust port of the reaction chamber to block the exhaust channel (since the sample volume is quantitative, it can at least reach near the exhaust port of the reaction chamber). This also helps the sample to be tested stay more stably in the reaction chamber after entering the reaction chamber, making backflow difficult to occur and preventing it from flowing to other chambers, thus ensuring that each step of the reaction can be completed smoothly until the final accurate detection is realized.

It can be seen that the valve block can play a variety of roles, such as controlling the communication state between the liquid storage chamber and the reaction chamber, preventing the sample from flowing out of the reaction chamber, etc.

In some embodiments, the lipid substances include n-hexadecane, n-octadecane, paraffin, stearic acid, palmitic acid, etc.

In some embodiments, the valve block is paraffin.

Paraffin can achieve phase change at a specific temperature threshold between 40-90 °C.

Further, when used for detecting different target nucleic acids in pre-processed samples, the liquid storage chamber does not need to be filled with lysis solution, and a valve block may not be arranged between the liquid storage chamber and the reaction chamber. The liquid storage chamber and the reaction chamber are in a fluid communication state, and the reagents in the liquid storage chamber directly enter different reaction chambers under the action of gravity.

In some embodiments, the cross-section of the reaction chamber is drop-shaped, and the exhaust port is located at the top of the drop shape.

The drop-shaped cross-section of the reaction chamber helps the liquid paraffin to collect upward, making it easier to condense near the exhaust port.

It can be understood that the solid reagents pre-embedded in the liquid storage chamber and the reaction chamber can be flexibly set. Different reaction reagents are pre-embedded in different chambers. For example, amplification primers for targets are pre-embedded in the liquid storage chamber, and detection probes for different genotypes are pre-embedded in each reaction chamber, so that genotyping detection of genes can be realized.

In some embodiments, the sample-adding area further includes a tube cap, which is used for sealing the microfluidic chip.

The nucleic acid typing detection chip provided by the present invention includes a gravity-driven typing chip and an asymmetric pressure-driven typing chip. The tube cap of the gravity-driven typing chip can be made in the form of a plug, or the cover surface of the tube cap is concave downward, etc. When the tube cap is tightened to seal the microfluidic chip, it can provide a certain downward pressure, prompting the sample near the sample-adding port to completely flow into the liquid storage chamber, and making the amplification product in the liquid storage chamber in a relatively high-pressure state.

The gravity-driven typing chip refers to a chip where the sample can directly and automatically reach the reaction chamber from the liquid storage chamber under the drive of gravity.

The asymmetric pressure-driven typing chip is similar to the gravity-driven typing chip, but the difference is that its tube cap can apply pressure to the chip after the chip is sealed to form instantaneous asymmetric pressure, so that the sample can quickly flow into the reaction chamber from the sample inlet channel under the drive of instantaneous asymmetric air pressure, but cannot flow out of the reaction chamber from the exhaust channel.

After the tube cap of the asymmetric pressure-driven typing chip closes the chip, it can also apply pressure to the inside of the chip to generate asymmetric pressure inside the chip, and promote the sample to flow from the liquid storage chamber into the reaction chamber through the sample inlet channel.

In existing microfluidic chips, the flow of the sample requires continuous external driving to realize the directional flow of the sample, which has high requirements for instruments, equipment, and driving stability. The asymmetric pressure-driven typing chip provided by the present invention does not require any external driving when the liquid flows in it, and can be realized only by the instantaneous asymmetric pressure generated by screwing or pressing the tube cap itself.

In the asymmetric pressure-driven typing chip, by screwing or pressing the tube cap, pressure is applied to the sealed system, and the reaction chamber is provided with a sample inlet channel and an exhaust channel. The exhaust channel is a multi-turn tortuous flow channel with a small pore size. Since the pore size of the sample inlet channel is larger than that of the exhaust channel, instantaneous asymmetric pressure is formed to drive the liquid to move. While the sample flows into the reaction chamber through the sample inlet channel, the gas inside the reaction chamber can be connected to the liquid storage chamber through the exhaust channel. At this time, the air pressure inside the microfluidic chip is balanced, and the sample entering the reaction chamber is also difficult to flow out of the reaction chamber, and backflow is difficult to occur, forming a circular closed environment, which effectively avoids aerosol contamination.

The main flow channel of the microfluidic chip and the sample inlet channel of the reaction chamber, which are used for sample flow, have pore sizes set to enable the liquid to flow smoothly.

The cross-sectional area of the exhaust channel is smaller than that of the sample inlet channel. The exhaust channel with a small cross-sectional area can only allow gas to pass through and is not smooth for liquid discharge. When a certain pressure is applied, instantaneous asymmetric pressure will be formed. After the reaction chamber is filled with the sample, the sample will be blocked when continuing to advance, and it is difficult to flow out from the exhaust channel of the reaction chamber, and will remain in the reaction chamber to complete the reaction and achieve accurate detection.

The tube cap is used for sealing the microfluidic chip and providing instantaneous pressure. The tube cap can be made in the form of spiral pressing, or the cover surface of the tube cap is embedded with a piston, etc. When the tube cap seals the microfluidic chip, or after the chip is sealed, by screwing or pressing the tube cap, a certain air pressure is provided to the inside of the chip. Since the pore size of the sample inlet channel is significantly larger than that of the exhaust channel and the exhaust channel is a multi-turn tortuous flow channel with a small pore size, instantaneous asymmetric pressure is formed, which promotes the transfer of liquid from the liquid storage chamber to the reaction chamber.

Further, after the tube cap closes the chip, pressure can be applied to the inside of the chip by screwing or pressing the tube cap.

Further, a pushable airbag is arranged at the top of the tube cap; after closing the chip, pushing the airbag downward can apply pressure to the inside of the chip to generate asymmetric pressure inside the chip, and promote the sample to flow from the liquid storage chamber into the reaction chamber. The airbag can ensure that the tube cap maintains a sealed state for the chip, even when the airbag is moving.

In some embodiments, the spiral tube cap is detachably connected to the liquid storage chamber, and while sealing the system, it provides air pressure for the chip system to drive the liquid in the liquid storage chamber to transfer to the reaction chamber.

The detection method using the gravity-driven typing chip provided by the present invention includes the following steps:
(1) Add the sample to be tested containing lysis solution into the gravity-driven typing chip from the sample-adding port, and cover the tube cap;
(2) Vertically insert the microfluidic chip into the detection device;
(3) The sample to be tested undergoes lysis or nucleic acid amplification in the liquid storage chamber;
(4) Heat to convert the valve block from a solid state to a liquid state;
(5) The sample to be tested is driven by gravity to enter each reaction chamber for reaction;
(6) Read the detection result.

In step (3), the nucleic acid amplification is isothermal amplification, and a heating device is required to provide the liquid storage chamber with a suitable temperature for isothermal amplification (such as 20-65 °C).

In step (4), the detection device needs to heat the valve block (paraffin) (such as 40-90 °C) to reach its phase change temperature and melt into a liquid.

The gravity-driven typing chip provided by the present invention, through the clever design of flow channel connection and chamber position, does not require additional driving. The liquid can realize the sequential and independent amplification and detection reactions of the sample to be tested according to its own gravity. The whole process is easy to operate and has a fast reaction speed, and can effectively realize high-throughput detection of pathogens; in addition, the amplification/detection reagents of the microfluidic chip are all dry reagents, which can realize the room-temperature storage and transportation of the chip, avoiding the limitations of cold-chain transportation and −20 °C storage.

The detection method using the asymmetric pressure-driven typing chip provided by the present invention includes the following steps:
(1) Add the sample to be tested containing lysis solution into the asymmetric pressure-driven typing chip from the sample-adding port, and cover the tube cap;
(2) Vertically insert the asymmetric pressure-driven typing chip into the detection device;
(3) The sample to be tested undergoes lysis or nucleic acid amplification in the liquid storage chamber;
(4) Heat to convert the valve block from a solid state to a liquid state;
(5) Press the airbag on the tube cap, and the sample to be tested is driven by asymmetric pressure to enter each reaction chamber for reaction;
(6) Read the detection result.

In step (3), the nucleic acid amplification is isothermal amplification, and a heating device is required to provide the liquid storage chamber with a suitable temperature for isothermal amplification (such as 20-65 °C).

In step (4), the detection device needs to heat the valve block (paraffin) (such as 40-90 °C) to reach its phase change temperature and melt into a liquid.

The asymmetric pressure-driven typing chip provided by the present invention, through the clever design of flow channel connection and chamber position, applies pressure to the sealed system by screwing the tube cap. At the same time, since the sample inlet channel is significantly larger than the exhaust channel, instantaneous asymmetric pressure is generated, realizing the sequential and independent amplification and detection reactions of the sample to be tested. The whole process is not only easy to operate and has a fast reaction speed, but also the chip is designed with a fully closed structure, which effectively avoids aerosol contamination and can effectively realize high-throughput detection of pathogens; in addition, the amplification/detection reagents of the microfluidic chip are all dry reagents, which can realize the room-temperature storage and transportation of the chip, avoiding the limitations of cold-chain transportation and −20 °C storage.

The nucleic acid multiplex detection microfluidic chip provided by the present invention has the following beneficial effects:
On the basis of ensuring detection precision, the structure and operation process are greatly simplified, the operation is more convenient, the cost is lower, and it is suitable for large-scale promotion and application;
A gravity-driven microfluidic chip is provided, which mainly has the following beneficial effects:
(1) No additional driving device is required, and the sample can realize autonomous transfer to the reaction chamber under the drive of gravity;
(2) A fluid channel is designed between the reaction chamber and the detection chamber. The fluid channel is a multi-turn tortuous flow channel with a small pore size, long length, and high position. Under the action of gravity alone, the sample cannot enter the fluid channel, so that after the reaction chamber is filled with the sample, the sample will be blocked when continuing to advance, and it is difficult to flow out from the fluid channel at the top of the reaction chamber. All the samples remain in the reaction chamber to complete the nucleic acid amplification reaction;
(3) The structure of the sample inlet channel is designed, so that the sample inlet of the reaction chamber is located at the lowest point of the sample inlet channel, so that the sample cannot overcome the gravity and the pressure caused by the sample inlet channel to return to the main flow channel, and all remain in the reaction chamber, preventing sample backflow;
(4) The reaction chamber does not need to be provided with an exhaust channel. The multi-turn tortuous fluid channel with a small pore size can not only act as a valve for temporarily blocking the liquid flow between the reaction chamber and the detection chamber, but also act as a temporary exhaust channel for the reaction chamber. The reaction chamber does not need to be additionally provided with an exhaust channel and a waterproof and breathable membrane, so the structure is simpler and the cost is lower;
(5) A simple pressure device is designed. After the sample completes the nucleic acid amplification in the reaction chamber, the sample can enter the detection chamber from the reaction chamber by pressing the pressure device, realizing the controllable transfer of the sample in different chambers; thus, it can adapt to more simple detection environments, realize low-cost integration, and is suitable for large-scale promotion and application.

### Beneficial Effects

(1) No additional driving device is required for the sample in the liquid storage chamber, and the sample can realize autonomous transfer to the reaction chamber under the drive of gravity or asymmetric pressure;
(2) The reaction chamber does not need to be connected to the external atmosphere. The exhaust channel of the reaction chamber is directly connected to the liquid storage chamber, and the atmosphere pressed out of the reaction chamber is discharged back to the liquid storage chamber. At this time, the air pressure inside the microfluidic chip is balanced, and the sample entering the reaction chamber is also difficult to flow out of the reaction chamber, and backflow is difficult to occur, forming a circular closed environment;
(3) On the basis of ensuring detection precision, the structure and operation process are greatly simplified. The operation is simple, the reaction speed is fast, and the chip adopts a fully closed structure design, which effectively avoids aerosol contamination. It can be extended to the rapid and visual detection of other pathogen nucleic acids in respiratory tract, reproductive tract samples, etc., and is suitable for large-scale promotion and application.

### BRIEF DESCRIPTION OF DRAWINGS

FIG. 1 is a back view of the gravity-driven microfluidic chip in Example 1;

FIG. 2 is a front view of the gravity-driven microfluidic chip in Example 1;

FIG. 3 is a perspective view (with the back facing up) of the gravity-driven microfluidic chip in Example 1;

FIG. 4 is a perspective view of the gravity-driven microfluidic chip in Example 1;

FIG. 5 is a schematic structural diagram of the detection unit of the gravity-driven microfluidic chip in Example 1;

FIG. 6 is a back view of the simple gravity-driven microfluidic chip in Example 2;

FIG. 7 is a front view of the simple gravity-driven microfluidic chip in Example 2;

FIG. 8 is a perspective view of the simple gravity-driven microfluidic chip in Example 2;

FIG. 9 is a front view of the centrifugal microfluidic chip in Example 4 (first type of exhaust channel, including a chip body and a sub-chip);

FIG. 10 is a back view of the centrifugal microfluidic chip in Example 4 (first type of exhaust channel, including a chip body and a sub-chip);

FIG. 11 is a perspective view of the centrifugal microfluidic chip in Example 4 (first type of exhaust channel, including a chip body and a sub-chip);

FIG. 12 is a schematic structural diagram of the detection unit in the centrifugal microfluidic chip in Example 4 (first type of exhaust channel);

FIG. 13 is a front view of the centrifugal microfluidic chip in Example 5 (first type of exhaust channel, no sub-chip);

FIG. 14 is a back view of the centrifugal microfluidic chip in Example 5 (first type of exhaust channel, no sub-chip);

FIG. 15 is a perspective view of the centrifugal microfluidic chip in Example 5 (first type of exhaust channel, no sub-chip);

FIG. 16 is a schematic structural diagram of the detection unit in the centrifugal microfluidic chip in Example 5 (first type of exhaust channel, no sub-chip);

FIG. 17 is a front view of the centrifugal microfluidic chip in Example 6.1 (second type of exhaust channel);

FIG. 18 is a back view of the centrifugal microfluidic chip in Example 6.1 (second type of exhaust channel);

FIG. 19 is a perspective view of the centrifugal microfluidic chip in Example 6.1 (second type of exhaust channel);

FIG. 20 is a schematic structural diagram of the detection unit in the centrifugal microfluidic chip in Example 6 (second type of exhaust channel).

FIG. 21 is a perspective view of the centrifugal microfluidic chip in Example 6.2 (second type of exhaust channel).

FIG. 22A is a schematic structural diagram of the detection unit in the centrifugal microfluidic chip in Example 6.2 (second type of exhaust channel).

FIG. 22B is the principle for the liquid from reaction chamber to entering into the detection chamber through the first channel and the air in the detection chamber to exhaust into the air channel.

FIG. 23 is a front view of a variant of the centrifugal microfluidic chip (two-split disk chip) in Example 6.2 (second type of exhaust channel);

FIG. 24 is a front view of a variant of the centrifugal microfluidic chip (four-split disk chip) in Example 6.2 (second type of exhaust channel);

FIG. 25 is a front view of the gravity-driven typing chip with four reaction chambers in Example 9;

FIG. 26 is a back view of the gravity-driven typing chip with four reaction chambers in Example 9;

FIG. 27 is a top view of the gravity-driven typing chip with four reaction chambers in Example 9;

FIG. 28 is a schematic structural diagram of the sample-adding area of the gravity-driven typing chip with four reaction chambers in Example 9;

FIG. 29 is a schematic cross-sectional structural diagram of the sample-adding area of the gravity-driven typing chip with four reaction chambers in Example 9;

FIG. 30 is a schematic structural diagram of the detection area of the gravity-driven typing chip with four reaction chambers in Example 9;

FIG. 31 is a structural diagram of the gravity-driven typing chip with six reaction chambers arranged radially in Example 9;

FIG. 32 is a structural diagram of the gravity-driven typing chip with four reaction chambers arranged radially in Example 9;

FIG. 33 is a structural diagram of the gravity-driven typing chip with four reaction chambers arranged horizontally in Example 9.

FIG. 34 is a structural diagram of the tube cap of the asymmetric pressure-driven typing chip in Example 13;

FIG. 35 is a schematic cross-sectional structural diagram of the tube cap of the asymmetric pressure-driven typing chip in Example 13;

FIG. 36 is a front view of the asymmetric pressure-driven typing chip with four reaction chambers in Example 13;

FIG. 37 is a back view of the asymmetric pressure-driven typing chip with four reaction chambers in Example 13;

FIG. 38 is a top view of the asymmetric pressure-driven typing chip with four reaction chambers in Example 13;

FIG. 39 is a schematic structural diagram of the sample-adding area of the asymmetric pressure-driven typing chip with four reaction chambers in Example 13;

FIG. 40 is a schematic cross-sectional structural diagram of the sample-adding area of the asymmetric pressure-driven typing chip with four reaction chambers in Example 13;

FIG. 41 is a schematic structural diagram of the detection area of the asymmetric pressure-driven typing chip with four reaction chambers in Example 13;

FIG. 42 is a structural diagram of the asymmetric pressure-driven typing chip with six reaction chambers arranged radially in Example 13;

FIG. 43 is a structural diagram of the asymmetric pressure-driven typing chip with four reaction chambers arranged radially in Example 13;

FIG. 44 is a structural diagram of the asymmetric pressure-driven typing chip with four reaction chambers arranged horizontally in Example 13.

### DETAILED DESCRIPTION

The structures involved in the present invention or the technical terms used are further explained below. Unless otherwise specified, they shall be understood and explained according to the general terms commonly used in the art.

In the description of the present invention, it should be understood that the terms “upper”, “lower”, “front”, “rear”, “left”, “right”, “vertical”, “horizontal”, “top”, “bottom”, “inner”, “outer” and other indicated orientations or positional relationships are based on the orientations or positional relationships shown in the accompanying drawings, which are only for the convenience of describing the present invention and simplifying the description, rather than indicating or implying that the indicated device or element must have a specific orientation, be constructed and operated in a specific orientation, and thus cannot be understood as limiting the present invention. In addition, the terms “first”, “second” and the like are only used for descriptive purposes, and cannot be understood as indicating or implying relative importance or implicitly indicating the number of indicated technical features. Thus, the features defined with “first”, “second” and the like may explicitly or implicitly include one or more of these features. In the description of the present invention, unless otherwise specified, “plurality” means two or more.

In the description of the present invention, it should be noted that unless otherwise clearly specified and limited, the terms “connected” and “connected” should be understood in a broad sense, for example, they can be fixed connection, detachable connection, or integrated connection; they can be mechanical connection or electrical connection; they can be direct connection or indirect connection through an intermediate medium, and can be internal communication between two elements. For those of ordinary skill in the art, the specific meanings of the above terms in the present invention can be understood according to specific situations.

### Detection

Detection means assaying or testing the presence or absence of a substance or a material, including but not limited to, a chemical substance, an organic compound, an inorganic compound, a metabolite, a drug or a drug metabolite, an organic tissue or a metabolite of an organic tissue, a nucleic acid, a protein, or a polymer. In addition, detection means testing the quantity of a substance or a material. Further, assay also means immunoassay, chemical assay, enzyme assay, and the like.

### Sample

In the present invention, the detected sample or specimen includes biological fluids. The initial state of the sample can be liquid, solid, or semi-solid. Solid or semi-solid samples can be converted into liquid samples by any appropriate method, such as mixing, mashing, maceration, incubation, dissolution, enzymatic hydrolysis, etc., and then poured into the collection chamber, and the detection element is used to detect whether the sample contains the analyte. Samples can be taken from humans, animals, plants, nature, etc. Samples taken from humans can be, for example, liquid samples such as blood, serum, urine, cerebrospinal fluid, sweat, lymph, saliva, gastric juice; solid or semi-solid samples such as feces, hair, cutin, tartar, fingernails/toenails. Samples taken from plants can be, for example, solid samples such as roots, stems, leaves; liquid or semi-solid samples such as tissue fluid, cell sap prepared from roots, stems, leaves. Samples taken from nature can be, for example, liquid samples such as rainwater, river water, seawater, groundwater; solid or semi-solid samples such as soil, rocks, minerals, petroleum.

### Downstream and Upstream

Downstream or upstream is divided according to the flow direction of a liquid. Generally, a liquid flows from an upstream area to a downstream area. The downstream area receives a liquid from the upstream area, and a liquid may also flow to the downstream area along the upstream area. Here, downstream or upstream is generally divided according to the flow direction of a liquid, and for example, on some materials where capillary force is utilized to promote the flow of a liquid, a liquid may overcome gravity to flow towards an opposite direction to the gravity; and at this time, downstream or upstream is divided according to the flow direction of a liquid.

### Gas Communication or Liquid Communication

Gas communication or liquid communication means that a liquid or a gas can flow from one place to another. In the flow process, the liquid or the gas may pass through some physical structures that play a guiding role. The so-called “passage through physical structures” means that the liquid passes through the surface of these physical structures or the internal space of these structures and flows to another place passively or actively, where the passive flow is usually caused by external forces, such as the flow under the capillary action. The flow here may also be a flow due to self-action (gravity or pressure) of a liquid or a gas, and may also be a passive flow. The communication here does not mean that a liquid or a gas is necessarily present, but only in some cases indicates a connection relationship or state between two objects. If a liquid is present, it can flow from one object to another object. Here, it means a state in which two objects are connected. On the contrary, if the state of liquid communication or gas communication is not present between two objects, and if a liquid exists in or on one object but cannot flow into or onto another object, such a state is a non-communication, non-liquid communication, or non-gas communication state.

### Nucleic Acid

The term “nucleic acid” includes any compound and/or substance that can be incorporated into an oligonucleotide chain. Exemplary nucleic acids used in the present application include, but are not limited to, DNA, RNA including messenger RNA (mRNA), hybrids thereof, RNAi inducers, RNAi agents, siRNA, shRNA, miRNA, antisense RNA, ribozymes, catalytic DNA, RNA inducing triple helix formation, aptamers, vectors, etc.

As used herein, the term “nucleic acid” includes one or more of the following types: polydeoxyribonucleotides (containing 2-deoxy-D-ribose), polyribonucleotides (containing D-ribose), and any other type of polynucleotide, which is an N-glycoside of purine or pyrimidine bases or modified purine or pyrimidine bases (including abasic sites). The term “nucleic acid”, as used herein, also includes covalently bonded polymers of ribonucleosides or deoxyribonucleosides, which are usually bonded through phosphodiester bonds between subunits, but in some cases through thiophosphates, methylphosphonates, etc. “Nucleic acid” includes single-stranded and double-stranded DNA as well as single-stranded and double-stranded RNA. Exemplary nucleic acids include, but are not limited to, gDNA; hnRNA; mRNA; rRNA, tRNA, microRNA (miRNA), small interfering RNA (siRNA), small nucleolar RNA (snoRNA), small nuclear RNA (snRNA), small temporal RNA (stRNA), etc., and any combination thereof.

### Nucleic Acid Multiplex Detection Microfluidic Chip

The nucleic acid multiplex detection microfluidic chip provided by the present invention is composed of any number of detection units. Each detection unit can be used to detect one or more target nucleic acids. Therefore, this chip is mainly used to detect target nucleic acids in samples. In fact, it can be understood that the microfluidic chip provided by the present invention is not only used for nucleic acid detection, but also can be used for the detection of any other target substances in samples. For example, the reaction chamber can be used for the pretreatment of target substances in samples, and the detection chamber can be used for the detection of target substances in samples.

In some embodiments, the nucleic acid multiplex detection microfluidic chip provided by the present invention is, for example, a gravity-driven microfluidic chip or a centrifugal microfluidic chip.

In some embodiments, the gravity-driven microfluidic chip refers to a chip where the sample can directly reach a specific chamber from the sample area under the action of gravity without any external driving force. For example, when used for nucleic acid multiplex detection, the sample can directly reach the reaction chamber from the sample area under the action of gravity.

In some embodiments, the centrifugal microfluidic chip refers to a chip where the sample needs to reach a specific chamber under the drive of centrifugal force. For example, when the sample is located in the sample area, it can enter the reaction chamber under the drive of centrifugal force; when the sample is in the reaction chamber, it can enter the detection chamber under the drive of centrifugal force.

In some embodiments, the centrifugal microfluidic chip can be a chip where the sample enters the reaction chamber under the action of gravity and then enters the detection chamber under the drive of centrifugal force. In this case, the centrifugal microfluidic chip also belongs to a type of gravity-driven microfluidic chip.

### Nucleic Acid Typing Detection Microfluidic Chip

The nucleic acid typing detection microfluidic chip refers to a microfluidic chip that can simultaneously detect multiple genotypes of the same target nucleic acid or multiple target nucleic acids of the same sample.

The nucleic acid typing detection microfluidic chip provided by the present invention is, for example, a gravity-driven typing chip, an asymmetric pressure-driven typing chip, etc. When the liquid flows in it, no external driving is required, and it can be realized only by the instantaneous asymmetric pressure generated by screwing or pressing the tube cap itself. The gravity-driven typing chip refers to a chip where the sample can directly and automatically reach the reaction chamber from the liquid storage chamber under the drive of gravity, but cannot flow out of the reaction chamber from the exhaust channel. The asymmetric pressure-driven typing chip is similar to the gravity-driven typing chip, but the difference is that its tube cap can apply pressure to the chip after the chip is sealed to form instantaneous asymmetric pressure, so that the sample can quickly flow into the reaction chamber from the sample inlet channel under the drive of instantaneous asymmetric air pressure, but cannot flow out of the reaction chamber from the exhaust channel.

### Nucleic Acid Multiplex Detection

The nucleic acid multiplex detection described in the present invention means that nucleic acid needs to be detected in two steps. After the target nucleic acid in the sample is amplified, it cannot be directly detected due to unclear signals or other reasons, and a second step of signal amplification is required to realize detection.

Therefore, to meet the needs of nucleic acid multiplex detection, each detection unit in the chip needs to contain a reaction chamber and a detection chamber, so that the two steps of nucleic acid detection can be controlled respectively. It is necessary to realize the controllable transfer of the sample between the reaction chamber and the detection chamber, and prevent cross-influence caused by sample backflow.

### Reaction Chamber

The nucleic acid detection microfluidic chip provided by the present invention includes any number of detection units, and each detection unit includes a reaction chamber and a detection chamber.

When used for nucleic acid multiplex detection, if the reaction chamber is used for amplifying the target nucleic acid in the sample, nucleic acid amplification reagents need to be pre-embedded in the reaction chamber; the nucleic acid amplification reagents can be liquid reagents or solid reagents, preferably solid amplification reagents prepared by freeze-drying or drying. The nucleic acid amplification reagents are common temperature-variable or isothermal nucleic acid amplification reagents, including but not limited to ERA (Enzymatic Recombinase Amplification), RPA (Recombinase Polymerase Amplification), LAMP (Loop-mediated Isothermal Amplification), NEAR (Nicking enzyme-Assisted Reaction), NASBA (Nuclear acid sequence-based amplification), HDA (helicase-dependent amplification), TMA, SAT, RCR, etc. The form of the solid amplification reagent includes, but is not limited to, spherical, powdery, flaky, or blocky preparations in the form of freeze-drying, drying, air-drying, etc.

In some embodiments, the reaction chamber can be used as an amplification chamber or a reaction chamber, such as an ERA amplification chamber, an ERA reaction chamber, an RPA amplification chamber, an RPA reaction chamber, etc.

In some embodiments, the reaction chamber is not only used for amplifying the target nucleic acid in the sample. When the microfluidic chip is used for detecting any other target substances in the sample, such as proteins, chemical molecules, etc., pretreatment reagents for the sample, including reagents for extraction, purification, derivatization, signal amplification of the sample, etc., can be pre-embedded in the reaction chamber.

In some embodiments, the reaction chamber can also be used for the lysis treatment of the sample as a lysis reaction chamber, and the detection chamber can be used for the amplification and detection of the target nucleic acid in the lysed sample as an amplification reaction chamber.

### Detection Chamber

When used for nucleic acid multiplex detection, if the reaction chamber is used for amplifying the target nucleic acid and nucleic acid amplification reagents are pre-embedded in the reaction chamber, the detection chamber is used for detecting the target nucleic acid and nucleic acid detection reagents are pre-embedded in the detection chamber.

In some embodiments, when used for nucleic acid multiplex detection, detection reagents need to be pre-embedded in the detection chamber; the detection reagents can be liquid reagents or solid reagents, preferably solid detection reagents prepared by freeze-drying or drying, including but not limited to spherical, powdery, flaky, or blocky preparations in the form of freeze-drying, drying, air-drying, etc.

In some embodiments, the detection reagents include detection primers, probes, and/or detection premix, and the detection premix includes enzymes and buffers required for detection.

In some embodiments, the detection reagents include, but are not limited to, various detection systems established based on ERA, RPA, LAMP, NEAR, NASBA, HAD, TMA, SAT, PCR, hybridization probe technology, CRISPR, Ago, RNase H, etc.

In some embodiments, the detection chamber can be used as a nucleic acid detection chamber, such as an ERA detection chamber, a CRISPR detection chamber, an Ago detection chamber, an RNase H detection chamber, etc.

In some embodiments, the detection chamber is not only used for detecting the target nucleic acid in the sample. When the microfluidic chip is used for detecting any other target substances in the sample, such as proteins, chemical molecules, etc., detection reagents for the target substances, including antibodies, antigens, fluorescent labels, chemiluminescent reagents, microspheres, etc., can be pre-embedded in the detection chamber.

### Fluid Channel

The reaction chamber and the detection chamber are connected through a fluid channel, which can be used for the circulation of liquid or gas, so that the liquid or gas in the reaction chamber can be transferred to the detection chamber, and the liquid or gas in the detection chamber can also be transferred to the reaction chamber.

In some embodiments, the fluid channel can be a channel of any shape, such as a straight line shape, an arc shape, etc. The pore size in the fluid channel can also be changed continuously, from thick to thin or from thin to thick, etc., which can be set according to actual needs. It is also possible to change the pore size of the fluid channel according to actual needs, for example, to make the pore size small enough to prevent the liquid from passing through while allowing the gas to remain circulating, so that only the gas can pass through and the liquid cannot pass through.

In some embodiments, for the gravity-driven microfluidic chip, when the fluid channel connects the reaction chamber and the detection chamber, it also needs to play the role of a valve. The sample should not enter the detection chamber when entering the reaction chamber, and can only enter the detection chamber under pressure. At this time, the fluid channel needs to be set as a fluid channel with a valve effect according to needs, such as reducing the pore size, extending the fluid channel, increasing the position of the fluid channel, setting it in an arched shape, setting a plurality of tortuous structures, etc., to increase the resistance of entering the detection chamber.

In some embodiments, for the gravity-driven microfluidic chip, the exhaust channel can also be arranged on the fluid channel, and the detection chamber is not provided with an exhaust channel. At this time, the resistance of the sample entering the detection chamber can also be increased, thereby realizing the controllable transfer of the sample between the reaction chamber and the detection chamber.

In some embodiments, for the centrifugal microfluidic chip, when the fluid channel connects the reaction chamber and the detection chamber, it can also play the role of a valve. By arranging the exhaust channel on the fluid channel, the detection chamber has no exhaust channel, so the sample cannot directly enter, and can only enter the detection chamber under the drive of centrifugal force.

In some embodiments, for the centrifugal microfluidic chip, the exhaust channel can also be set in a variety of ways, such as reducing the pore size, extending the fluid channel, increasing the position of the fluid channel, setting it in an arched shape, setting a plurality of tortuous structures, etc., to prevent the sample from entering the detection chamber, thereby playing the role of a valve.

### Exhaust Channel

The exhaust channel is used to balance the pressure inside the microfluidic chip. For example, the gas in the reaction chamber is discharged to allow the sample to smoothly enter the reaction chamber, and the other gas in the detection chamber is discharged to allow the sample to enter the detection chamber. Of course, the exhaust channel is not only used for gas flow. In some cases, for example, when the sample overflows, it may also enter the exhaust channel.

In existing nucleic acid detection microfluidic chips, in order to realize the controllable transfer of the sample between the reaction chamber and the detection chamber, it is usually necessary to set exhaust channels in the reaction chamber and the detection chamber respectively, so that the reaction chamber and the detection chamber can exhaust smoothly, and the sample can flow in smoothly.

In the microfluidic chip provided by the present invention, the reaction chamber and the detection chamber share one exhaust channel, that is, this exhaust channel can help the reaction chamber exhaust and also help the detection chamber exhaust, thereby controlling the flow of the liquid sample between the reaction chamber and the detection chamber.

The connection between the exhaust channel and the outside is covered with a waterproof and breathable membrane, which can block the passage of liquid but cannot block the passage of gas, thus realizing exhaust. However, when the waterproof and breathable membrane is wetted by water, it will block the exhaust holes of the waterproof and breathable membrane, making it lose its air permeability, so that the inside of the microfluidic chip becomes a fully closed system, which cannot exhaust, and the liquid also stops flowing in the system.

In some embodiments, this shared exhaust channel can be arranged at any position of the detection unit, as long as it can help the reaction chamber and the detection chamber exhaust smoothly. Of course, the position of the exhaust channel will directly affect the effect of the controllable transfer of the sample between the reaction chamber and the detection chamber. For example, the distance between the exhaust channel and the detection chamber is directly related to the resistance of the sample entering the detection chamber. Only when the exhaust channel is connected to the top of the detection chamber, when the liquid flows from the reaction chamber to the detection chamber and enters the detection chamber from the bottom of the detection chamber, the exhaust direction is also from the bottom of the detection chamber to the top of the detection chamber and then discharged from the exhaust channel. The exhaust direction is consistent with the liquid flow direction, so the resistance of the liquid sample entering the detection chamber is the smallest; when the exhaust channel is connected to other parts of the detection chamber, or not connected to the detection chamber but connected to the fluid channel, the discharged gas will be discharged from the bottom of the detection chamber to the top, then turned back, and pass through the liquid to reach the exhaust channel. That is to say, part of the exhaust process is opposite to the liquid flow direction. This turning back and passing through the liquid process will significantly increase the resistance of the sample entering the detection chamber. Therefore, changing the position of the exhaust channel will directly affect the effect of the controllable transfer of the sample between the reaction chamber and the detection chamber. For example, if the exhaust channel is too close to the reaction chamber and far from the detection chamber, the difficulty of transferring the sample into the detection chamber will be significantly increased. Another example is that if the exhaust channel is directly arranged on the reaction chamber or the sample inlet channel, this will not only make the difficulty of transferring the sample into the detection chamber too great, but also may cause the sample to enter the exhaust channel early and wet the waterproof and breathable membrane after entering the reaction chamber, making it unable to breathe again. The inside of the microfluidic chip is completely closed, and the liquid can no longer flow. Therefore, the position of this exhaust channel shared by the reaction chamber and the detection chamber is very critical. It must be ensured that it remains ventilated when the sample enters the reaction chamber, and it is not closed. Even if it is closed during the process, it can quickly resume ventilation.

In some embodiments, for the centrifugal microfluidic chip, the exhaust channel can be arranged in the fluid channel, at a distance from the detection chamber. Because even if the distance from the detection chamber is relatively large, which increases the difficulty of the sample entering the detection chamber, a larger driving force can still be provided by controlling the centrifugal force, thereby promoting the sample to smoothly enter the detection chamber from the reaction chamber. On the contrary, if the exhaust channel is too close to the detection chamber or directly located on the detection chamber, it is impossible to distinguish the force required for the sample to enter the reaction chamber or the detection chamber by controlling the centrifugal speed, resulting in the failure of the controllable transfer of the sample between the reaction chamber and the detection chamber. Therefore, in the present invention, the exhaust channel of the centrifugal microfluidic chip is preferably arranged in the fluid channel, which can be at a position close to the reaction chamber, close to the detection chamber, or in the middle of the fluid channel, and can be adjusted arbitrarily according to needs.

In some embodiments, for the gravity-driven microfluidic chip, the exhaust channel can be directly arranged in the detection chamber. Therefore, the fluid channel in this chip has a small pore size and is tortuous, which can provide sufficient resistance to enter the detection chamber. Moreover, the exhaust channel is arranged in the detection chamber, which makes it easier to control the flow of the sample and prevents the system from being closed due to the sample entering the exhaust channel. When the sample enters the detection chamber, the sample will enter the exhaust channel to close the system, so that the sample can no longer flow out of the detection chamber, thereby helping to realize the controllable transfer of the sample between the reaction chamber and the detection chamber.

### Combination of Fluid Channel and Exhaust Channel

The combination of the fluid channel and the exhaust channel described in the present invention refers to the combination of the design of various characteristics of the fluid channel (quantity, length, shape, height position, pore size, etc.) and the design of various characteristics of the exhaust channel (quantity, distance from the detection chamber, position, design of the exhaust port, direction of the exhaust channel, pore size, etc.).

In some embodiments, the pore size of the fluid channel is smaller than that of the sample inlet channel, and the exhaust channel is connected to the fluid channel. In some embodiments, the exhaust channel can be connected to any position of the fluid channel, such as the middle position of the fluid channel, the position close to the reaction chamber in the fluid channel, or the position closer to the detection chamber in the fluid channel.

In some embodiments, the exhaust channel can also be directly connected to the fluid channel. At this time, to make the sample enter the detection chamber from the reaction chamber, a larger driving force or pressure is required, and even additional external driving force may be required. It can be understood that the exhaust channel can be arranged at any position of the fluid channel, but the arrangement of the exhaust channel at different positions of the fluid channel will also affect the resistance of the sample entering the detection chamber. For example, if the exhaust channel is arranged in the fluid channel and is too close to the reaction chamber and too far from the detection chamber, the difficulty of transferring the sample into the detection chamber will be significantly increased, and it may even be difficult to enter the detection chamber. If the exhaust channel is too close to the detection chamber or directly arranged on the detection chamber, the resistance of the sample entering the detection chamber will be reduced, and the sample may also enter the detection chamber when entering the reaction chamber, making it difficult to realize the controllable transfer of the sample between the reaction chamber and the detection chamber. Therefore, the specific position of the exhaust channel can be selected according to the actual product situation and the magnitude of the force that can be provided by the pressure device or centrifugal force on the chip.

In some embodiments, the fluid channel is a tortuous flow channel, and the exhaust channel is connected to the fluid channel.

In some embodiments, the fluid channel is a spiral flow channel, and the exhaust channel is connected to the detection chamber. In some embodiments, the exhaust channel can be connected to any position of the detection chamber, such as the top of the detection chamber, the bottom of the detection chamber, the side part of the detection chamber, etc.

In some embodiments, the fluid channel is a multi-fold flow channel, the exhaust channel is connected to the fluid channel, the pore size of the fluid channel is smaller than that of the sample inlet channel, and the outlet of the exhaust channel is connected to the main flow channel.

In some embodiments, the fluid channel is a tortuous flow channel with a plurality of bent and folded structures, similar to the shape of the “Great Wall”. The exhaust channel is connected to the fluid channel, the pore size of the fluid channel is smaller than that of the sample inlet channel, and the outlet of the exhaust channel is connected to the main flow channel.

In some embodiments, the fluid channel is a tortuous flow channel with a plurality of bent and folded structures, the exhaust channel is connected to the top of the detection chamber, and the pore size of the fluid channel is smaller than that of the sample inlet channel (gravity-driven microfluidic chip).

In some embodiments, the exhaust channel is connected to the fluid channel, and the outlet of the exhaust channel is connected to the main flow channel or the external atmosphere (centrifugal microfluidic chip).

### Controllable Transfer Between Reaction Chamber and Detection Chamber

The controllable transfer between the reaction chamber and the detection chamber described in the present invention means that the sample can follow the pre-designed plan: when it is time to enter the reaction chamber, all the samples to be tested smoothly enter the reaction chamber and remain in the reaction chamber to complete the amplification reaction; when it is desired for the sample to flow out of the reaction chamber, the sample will smoothly flow out of the reaction chamber, enter the detection chamber through the fluid channel, and remain in the detection chamber to complete the detection reaction until the final smooth

All patents and publications mentioned in the specification of the present invention indicate that these are disclosed techniques in the art and can be used by the present invention All patents and publications cited herein are likewise listed in the references as if each publication is specifically and separately referenced. The present invention described herein may be implemented in the absence of any one or more elements, and one or more limitations, which are not specifically stated herein. For example, in each instance herein, the terms “comprising/including,” “consisting essentially of,” and “consisting of” may be replaced by either of the other 2 terms. The term “a/an” here merely means “one,” but does not exclude including 2 or more in addition to including only one. The terms and expressions employed herein are descriptive and are not limited thereto, and there is no intention herein to indicate that the terms and interpretations described herein exclude any equivalent features, but it can be noted that any appropriate changes or modifications can be made within the scope of the present invention and claims. It can be understood that the embodiments described in the present invention are preferred embodiments and features, and any person skilled in the art can make some modifications and changes based on the essence of the description of the present invention. These modifications and changes are also considered to be within the scope of the present invention and the scope limited by the independent claims and the dependent claims.

### SPECIFIC EMBODIMENTS

The preferred embodiments of the present invention will be described in further detail below with reference to the accompanying drawings. It should be noted that the following described embodiments are intended to facilitate the understanding of the present invention, and do not have any limiting effect on it. The raw materials and equipment used in the specific embodiments of the present invention are known products and can be obtained by purchasing commercial products.

### Detailed Description of Preferred Embodiments

### Example 1: Structure of the Gravity-Driven Microfluidic Chip

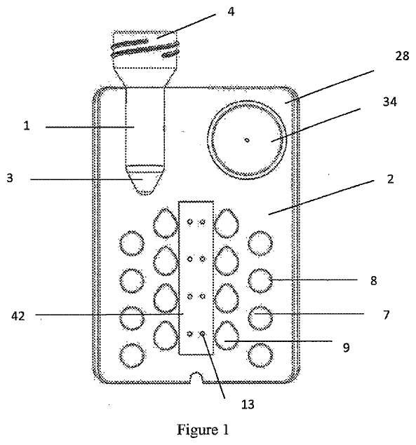

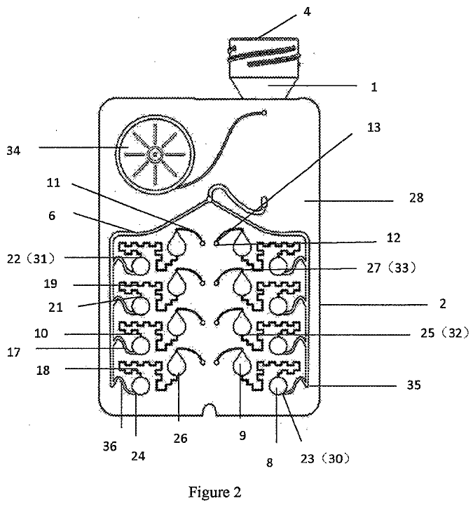

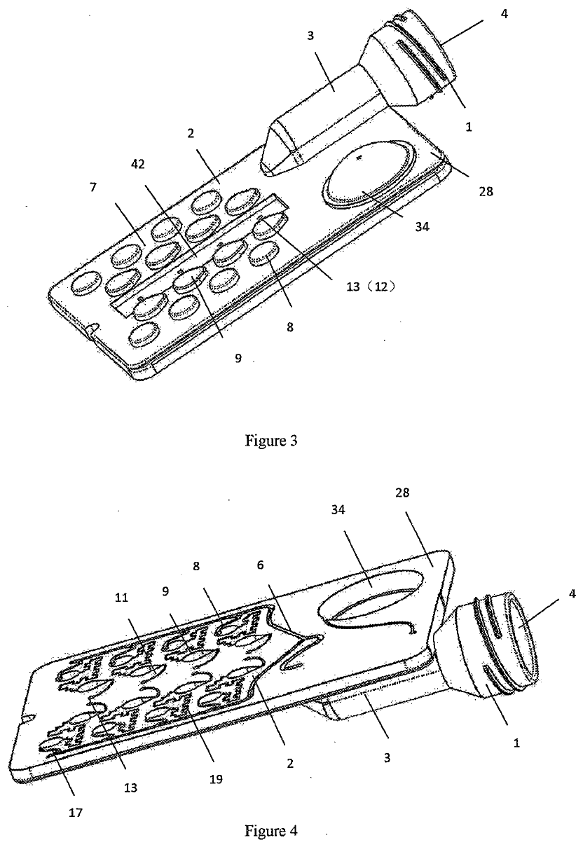

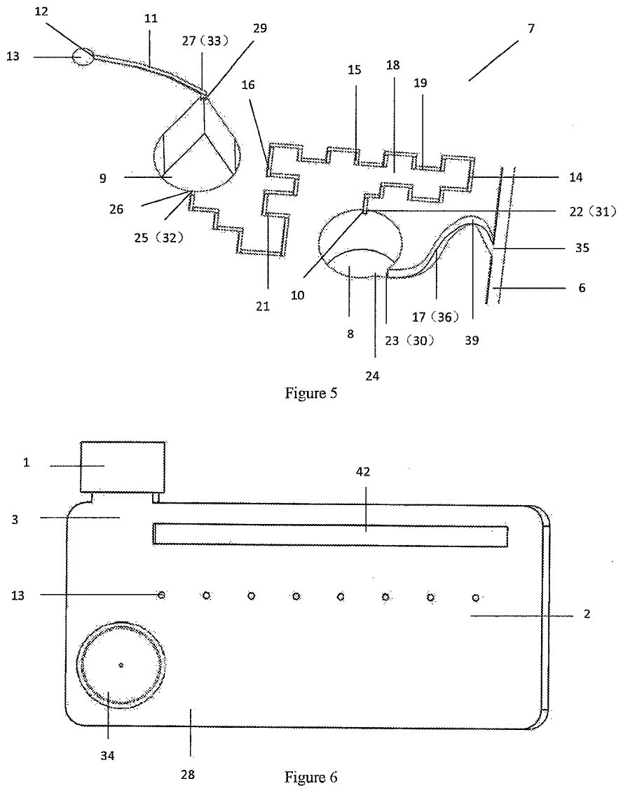

The structure of the nucleic acid multiplex detection microfluidic chip provided in this example is shown in FIGS. 1-5, where FIG. 1 is a back view of the nucleic acid multiplex detection microfluidic chip, FIG. 2 is a front view of the nucleic acid multiplex detection microfluidic chip; FIG. 3 is a perspective view (with the back facing up) of the nucleic acid multiplex detection microfluidic chip; FIG. 4 is a perspective view of the nucleic acid multiplex detection microfluidic chip; FIG. 5 is a schematic structural diagram of the detection unit.

As shown in FIGS. 1-4, the nucleic acid multiplex detection microfluidic chip 28 (hereinafter referred to as the chip 28) provided in this example includes a sample-adding area 1 and a detection area 2. The sample-adding area 1 includes a sample inlet 4, a sample pool 3, and a tube cap (not shown in the figure). The sample pool 3 is used for containing the sample to be tested. When the sample is added to the chip 28, it first enters the sample pool 3, then enters the main flow channel 6 under the action of gravity, and then flows into the detection area 2.

The detection area 2 can be provided with any number of groups of detection units 7 according to the project needs to realize the simultaneous detection of multiple targets. This example is preferably provided with ten groups of detection units 7, arranged in two columns, with five groups of detection units 7 in each column. Each group of detection units 7 includes a reaction chamber 8 and a detection chamber 9 connected to the reaction chamber 8. The reaction chamber 8 is connected to the main flow channel 6 through a sample inlet channel 17, and the reaction chamber 8 and the detection chamber 9 are in fluid communication through a fluid channel 19. The pore size of the fluid channel 19 is smaller than that of the sample inlet channel 17; under the action of gravity, the sample can flow from the sample-adding area 1 into the reaction chamber 8, but cannot flow into the fluid channel 19.

During the use of the chip 28, it needs to be kept in a vertical state, with the sample inlet 4 facing upward. The following description of the positional relationship of each flow channel and chamber in the chip 28 is based on the chip 28 being kept in a vertical state. Due to the pressure balance in the system, the sample in the sample-adding area 1 can realize autonomous transfer to the reaction chamber 8 under the drive of gravity without an external driving device, and this process is easy to realize. Once the sample enters the sample-adding area 1, it will flow downward by itself and enter the reaction chamber 8 through the sample inlet channel 17. The main flow channel 6 of the chip 28 and the sample inlet channel 17 of the reaction chamber 8 have a large pore size, which can enable the liquid to flow smoothly under the drive of gravity. However, the pore size of the fluid channel 19 is significantly smaller than these flow channels. The fluid channel 19 is a multi-turn tortuous flow channel with a small pore size. Under the action of gravity alone, the liquid cannot enter and flow smoothly. Therefore, after the reaction chamber 8 is filled with the sample, the sample will be blocked when continuing to advance, and it is difficult to flow out from the fluid channel 19 at the top 10 of the reaction chamber. All the samples will remain in the reaction chamber 8 to complete the nucleic acid amplification reaction. After the nucleic acid amplification reaction is completed, the sample can enter the detection chamber 9 through the fluid channel 19 only under the push of pressure, thereby ensuring accurate detection. The width of the main flow channel 6 and the plurality of sample inlet channels 17 in the chip 28 is 400 μm-800 μm. In this example, the width of the main flow channel is preferably 400-600 μm, and the depth is 400-600 μm. The width of the fluid channel is 50-300 μm. In this example, the width of the fluid channel is preferably 100 μm.

The detection chamber 9 is provided with an exhaust channel 11, and a waterproof and breathable membrane 13 is arranged at the outlet 12 of the exhaust channel 11, which is used for controlling the communication state between the detection chamber 9 and the external atmosphere; the reaction chamber 8 is not provided with an exhaust channel 11. The arrangement of the exhaust channel 11 and the waterproof and breathable membrane 13 of the detection chamber 9 can realize the discharge of gas from the reaction chamber 8 and the detection chamber 9 to the outside through the waterproof and breathable membrane 13, but the liquid cannot be discharged through the waterproof and breathable membrane 13. When the exhaust channel 11 is filled with liquid, the waterproof and breathable membrane 13 will lose its air permeability, so that the entire system is in a closed state, and the liquid in the system stops flowing. Therefore, the waterproof and breathable membrane 13 can help the sample in the detection chamber 9 establish an internal and external pressure balance, and can regulate the flow direction of the liquid. When the detection chamber 9 is filled with liquid and the waterproof and breathable membrane 13 is wetted through the exhaust channel 11, the system inside the chip 28 is in a closed state, so that the sample in the detection chamber 9 stops flowing and no backflow occurs. The waterproof and breathable membrane is made of a polymer material, such as high-grade synthetic rubber, which has good sealing performance, no back glue and residue, and does not react with reaction reagents or detection reagents. In this example, the waterproof and breathable membrane is preferably made of a material (manufacturer: Membrane; article number: VET022H60). The chip 28 can also be covered with a plastic sheet 42 at the position of the waterproof and breathable membrane. There is a gap between the plastic sheet 42 and the chip 28, which can not only connect the waterproof and breathable membrane 13 with the external gas, but also prevent the waterproof and breathable membrane 13 from falling off, and play a role in protecting the waterproof and breathable membrane 13.

In the chip 28, the reaction chamber 8 does not need to be provided with an exhaust channel. The multi-turn tortuous fluid channel 19 with a small pore size can act as a valve for temporarily blocking the liquid flow between the reaction chamber 8 and the detection chamber 9, but the reaction chamber 8 and the detection chamber 9 can always maintain ventilation. Therefore, the reaction chamber 8 and the detection chamber 9 can share the exhaust channel 11 located on the detection chamber 9 for exhaust, and the reaction chamber 8 does not need to be additionally provided with an exhaust channel 11 and a waterproof and breathable membrane 13. This design makes the structure simpler, the cost lower, the operation more convenient, and at the same time ensures the accuracy of the detection results.

As shown in FIG. 5, the fluid channel 19 includes a sample inlet section 14, a middle section 15, and a sample outlet section 16. The middle section 15 is arched upward, and the position of the middle section 15 is higher than that of the sample inlet section 14 and the sample outlet section 16. The upwardly arched middle section 15 of the fluid channel 19 increases the difficulty of the sample in the reaction chamber 8 entering the fluid channel 19 without additional pressure, which helps all the samples in the reaction chamber 8 remain in the reaction chamber 8 to complete the nucleic acid amplification reaction. The fluid channel 19 is located between the reaction chamber 8 and the detection chamber 9, and the middle section 15 of the fluid channel 19 extends toward the upper end 18 of the reaction chamber 8, which extends the length of the fluid channel 19 and increases the difficulty of the sample in the reaction chamber 8 entering the fluid channel 19 without additional pressure. The fluid channel 19 is a multi-turn tortuous flow channel, which is provided with at least one bent structure 21. The arrangement of a plurality of bent structures 21 in the fluid channel 19 can extend the length of the fluid channel 19 as much as possible, maximize the difficulty of the sample in the reaction chamber 8 entering the fluid channel 19 without additional pressure, and provide a guarantee for all the samples in the reaction chamber 8 to remain in the reaction chamber 8. The inlet 22 of the fluid channel 19 is located at the top 10 of the reaction chamber; the outlet 23 of the sample inlet channel 17 is located at the bottom 24 of the reaction chamber; the outlet 25 of the fluid channel 19 is located at the bottom 26 of the detection chamber; the inlet 27 of the exhaust channel 20 is located at the top 29 of the detection chamber.

The outlet 23 of the sample inlet channel 17 is equivalent to the sample inlet 30 of the reaction chamber 8, and the inlet 22 of the fluid channel 19 is equivalent to the sample outlet 31 of the reaction chamber 8. The sample inlet 30 is at the bottom of the reaction chamber 8, and the sample outlet 31 is at the top 10 of the reaction chamber. The purpose of this design is to ensure that when the sample enters the reaction chamber 8, it can smoothly exhaust to the sample outlet 31 and maintain pressure balance. At the same time, because the sample outlet 31 is at the top of the reaction chamber 8, the difficulty of the sample flowing out is increased, and the sample remains more stably in the reaction chamber 8. The outlet 25 of the fluid channel 19 is equivalent to the sample inlet 32 of the detection chamber 9, and the inlet 27 of the exhaust channel 11 is equivalent to the exhaust port 33 of the detection chamber 9. The sample inlet 32 is at the bottom 26 of the detection chamber 9, and the exhaust port 33 is at the top 29 of the detection chamber 9. The purpose of this design is to ensure that when the sample enters the detection chamber 9, it can smoothly exhaust to the exhaust port 33 and maintain pressure balance. Under the pressure of the pressure-applying area 34, the sample in the reaction chamber 8 enters the detection chamber 9 from the bottom 26 of the detection chamber through the fluid channel 19. Since the sample volume is usually sufficient, the sample can also enter the exhaust channel 11 and contact the waterproof and breathable membrane 13, making the waterproof and breathable membrane 13 lose its air permeability. The inside of the chip 28 is in a closed state as a whole, and the sample stops flowing, so that it is completely retained in the detection chamber 9 for the detection reaction and the detection of the target nucleic acid.

The position of the detection chamber 9 is higher than that of the reaction chamber 8, and the bottom 26 of the detection chamber 9 is not lower than the top 10 of the reaction chamber 8, which increases the difficulty of the sample in the reaction chamber 8 entering the detection chamber 9 without additional pressure, and helps prevent the sample in the reaction chamber 8 from flowing into the detection chamber 9 in advance.

As shown in FIG. 5, the sample inlet channel 17 of the chip 28 is in fluid communication with the branch port 35 of the main flow channel 6. The sample inlet channel 17 is an upwardly arched arc-shaped flow channel 36. The inlet 37 and outlet 38 of the sample inlet channel 17 are respectively located at the low points on both sides of the arc-shaped flow channel 36, and the inlet 37 is higher than the outlet 38; the highest point 39 of the arc-shaped flow channel 36 is not lower than the top 10 of the reaction chamber. The arc-shaped flow channel design of the sample inlet channel 17 enables the sample to easily enter the sample inlet channel 17 from the branch port 35 and then enter the reaction chamber 8 through the sample inlet channel 17. However, the sample entering the reaction chamber 8 is difficult to return to the main flow channel 6 from the sample inlet channel 17. Because the sample inlet 30 of the reaction chamber 8 is located at the lowest point of the sample inlet channel 17, the sample cannot overcome the gravity and the pressure caused by the upwardly arched arc-shaped flow channel 36 to return to the main flow channel 6, and can only remain in the reaction chamber 8, thereby preventing sample backflow in the reaction chamber 8.

When a specific volume of the sample to be tested is added from the sample-adding area 1, the cover of the sample-adding area 1 is closed, and the sample flows into the reaction chamber 8 of each detection unit 7 from the main flow channel 6 under the action of gravity. The sample inlet 30 of the reaction chamber 8 is at a low position, which enables the sample to be tested to quickly flow into the reaction chamber 8 and gradually fill it. However, at this time, the sample will not flow out of the reaction chamber 8 from the sample outlet 31, because the sample outlet 31 is at the top 10 of the reaction chamber 8, and the fluid channel 19 is a multi-turn tortuous flow channel with a small pore size, so the sample cannot flow into the fluid channel 19. At the same time, the sample is also difficult to return to the main flow channel 5 from the sample inlet channel 17, because the sample inlet 30 of the reaction chamber 8 is located at the lowest point of the sample inlet channel 17, and the highest point of the sample inlet channel 17 is higher than the top 10 of the reaction chamber 8. The sample cannot return to the main flow channel 6 under the action of gravity. Therefore, all the samples will remain stably in the reaction chamber 8 for reaction.

As shown in FIG. 2, the pressure-applying area 34 of the chip 28 is located on one side of the sample-adding area 1, and is used for increasing the pressure of the liquid in the reaction chamber 8 so that the liquid in the reaction chamber 8 flows into the detection chamber 8 through the fluid channel 19. The purpose of setting the pressure-applying area 34 is: after the sample completes the amplification and other reactions in the reaction chamber 8, the sample needs to completely enter the detection chamber 9 from the reaction chamber 8, and at this time, it is necessary to overcome a certain air pressure and the pressure caused by the multi-turn tortuous fluid channel 19 with a small pore size. The pressure-applying area 34 needs to be arranged on the upper left or upper right of the chip 28, and should also reach a specific volume (the specific volume can be calculated according to the size of the chip 28 and the required pressure to set the airbag 40 with a suitable volume), so that the pressing process provides a more accurate air pressure to drive the sample to be tested to enter the detection chamber 9 from the reaction chamber 8. The airbag 40 of the pressure-applying area 35 can be pressed manually or by a matching detection device providing a specific force to press the airbag 40. The pressure-applying area 34 can be connected to the sample pool 3 through a shunt channel 41. By pressing, air is discharged from the shunt channel 41 to the sample pool 3 and into the chip 28.

As shown in FIG. 5, the cross-section of the reaction chamber 8 is circular. The circular cross-section can enable the reaction chamber 8 to contain more samples. The cross-section of the detection chamber 9 is drop-shaped, and the exhaust port 33 is located at the top of the drop shape. The drop-shaped cross-section of the detection chamber 9 helps the gas or liquid in the detection chamber 9 to collect upward, making it easier to enter the exhaust channel 11. The solid reagents pre-embedded in the reaction chamber 8 and the detection chamber 9 can be flexibly set. Different reaction reagents are pre-embedded in different chambers. For example, amplification primers for targets are pre-embedded in the reaction chamber 8, and detection probes for different targets are pre-embedded in the detection chamber 9.

Nucleic acid amplification reagents need to be pre-embedded in the reaction chamber 8. The nucleic acid amplification reagents are solid amplification reagents prepared by freeze-drying or drying. The nucleic acid amplification reagents are common temperature-variable or isothermal nucleic acid amplification reagents, including but not limited to ERA (Enzymatic Recombinase Amplification), RPA (Recombinase Polymerase Amplification), LAMP (Loop-mediated Isothermal Amplification), NEAR (Nicking Enzyme-Assisted Reaction), NASBA (Nuclear acid sequence-based amplification), HDA (helicase-dependent amplification), etc.

The detection chamber 9 is used for the fluorescent detection reaction between the amplified sample and the detection reagent. The detection reagent includes, but is not limited to, various detection systems established based on ERA (Enzymatic Recombinase Amplification), RPA (Recombinase Polymerase Amplification), LAMP (Loop-mediated Isothermal Amplification), NEAR (Nicking Enzyme-Assisted Reaction), CRISPR (Clustered Regularly Interspaced Short Palindromic Repeats), Ago, RNase H, etc. The solid detection reagent specifically includes detection primers, probes, and/or detection premix, and the detection premix includes enzymes and buffers required for detection.

The body structure of the chip 28 provided in this example uses materials with low cost, easy processing performance, and good biocompatibility for chips, such as PMMA (Polymethyl Methacrylate), PP (Polypropylene), PC (Polycarbonate), etc., as the substrate. Channels and reaction tanks are directly made by laser etching technology, or structures are etched on the PMMA substrate, and then PDMS is used for reverse molding.

The chip 28 provided in this example, through the clever design of the structures of the sample inlet channel, fluid channel, chamber, and exhaust channel, does not require additional driving. The sample can enter the reaction chamber 8 by its own gravity without entering the detection chamber 9, and after completing the amplification reaction in the reaction chamber 8, it can enter the detection chamber 9 under the action of the simple pressure device 34, realizing the controllable transfer of the sample in different chambers without sample backflow.

### Example 2: Simple Gravity-Driven Microfluidic Chip

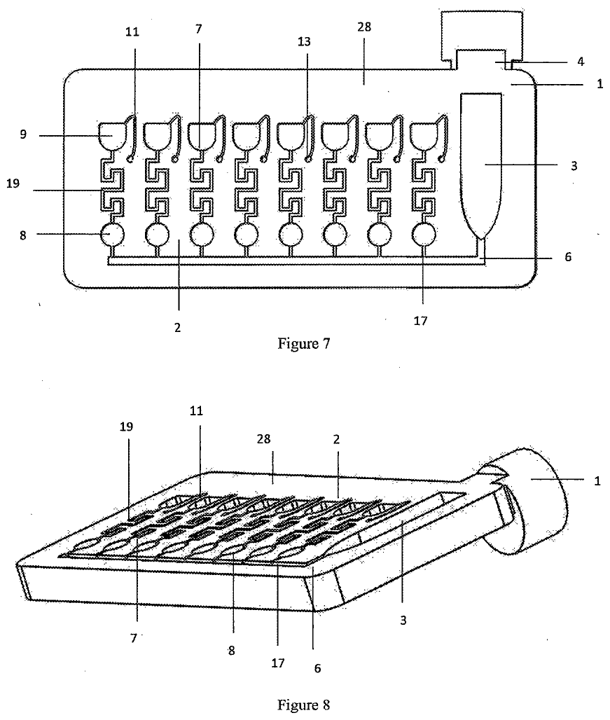

This example provides a simplified version of the nucleic acid multiplex detection microfluidic chip, as shown in FIGS. 6-8, where FIG. 6 is a back view of the simple nucleic acid multiplex detection microfluidic chip, FIG. 7 is a front view of the simple nucleic acid multiplex detection microfluidic chip, and FIG. 8 is a perspective view of the simple nucleic acid multiplex detection microfluidic chip.

The difference between the simple nucleic acid multiplex detection microfluidic chip provided in this example and the nucleic acid multiplex detection microfluidic chip in Example 1 is that only a single row of detection units 7 is designed, and the detection units 7 are placed vertically. The reaction chamber 8 is located below, the detection chamber 9 is located directly above the reaction chamber 8, and a fluid channel 19 is arranged between the reaction chamber 8 and the detection chamber 9; the sample pool 3 is located on one side of the detection unit 7. After the sample enters the sample pool, it will directly flow into the reaction chamber 8 under the action of gravity. Since the fluid channel 19 has a small pore size and a multi-turn tortuous design, the sample cannot enter the reaction chamber 8 under the action of gravity. After the amplification reaction is completed, the sample can smoothly enter the detection chamber 9 only by squeezing the pressure-applying area 34 (FIG. 6) on one side of the sample-adding area 1, and enter the exhaust channel 11 of the detection chamber 9 to contact the waterproof and breathable membrane 13, thereby closing the entire system, stopping the flow of the sample, and completing the detection in the detection chamber 9.

### Example 3: Usage Method of the Gravity-Driven Microfluidic Chip

The nucleic acid multiplex detection microfluidic chip 28 provided in Example 1 or Example 2 can be applied to various nucleic acid detection methods. This example takes the multiplex detection based on enzyme-catalyzed isothermal amplification technology as an example to illustrate the usage method of the chip 28:
Insert the chip 28 vertically into the detection device;
Transfer: Add the sample to be tested containing lysis solution into the chip 28 from the sample inlet 4. The sample flows into the reaction chamber 8 under the action of gravity, and tighten the tube cap to seal the chip;
The sample to be tested undergoes nucleic acid amplification in the reaction chamber 8;
After the nucleic acid amplification reaction is completed, squeeze the pressure-applying area 34 to make the sample to be tested after nucleic acid amplification enter the detection chamber 9 for reaction;
Read the result: After the reaction is completed, read the fluorescent signal through the device to obtain the detection result.

The technical features of the above-described embodiments can be combined arbitrarily. To simplify the description, all possible combinations of the technical features in the above-described embodiments are not described. However, as long as there is no contradiction between the combinations of these technical features, they should be considered as being within the scope described in this specification.

### Example 4: Centrifugal Microfluidic Chip with the First Type of Exhaust Channel Setting

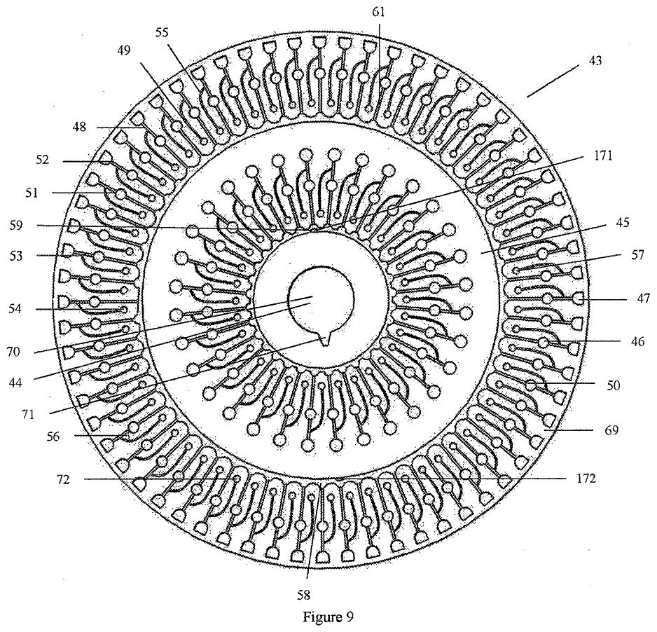

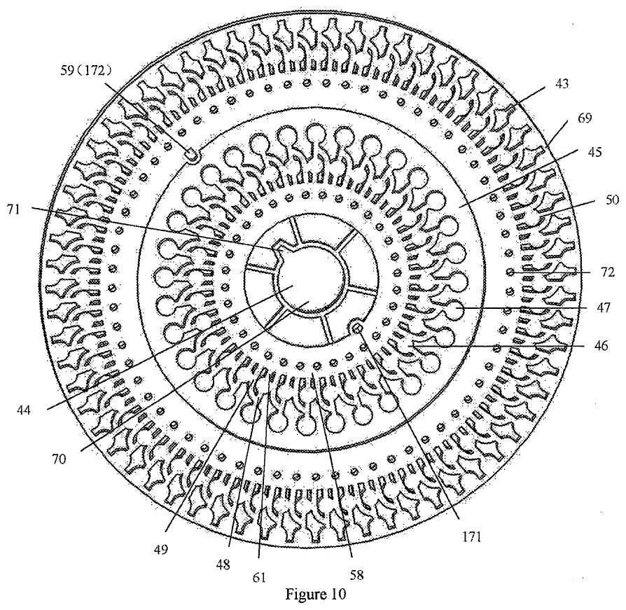

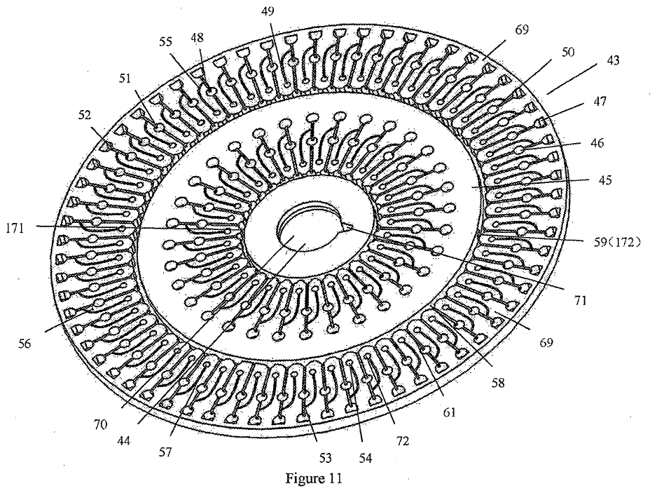

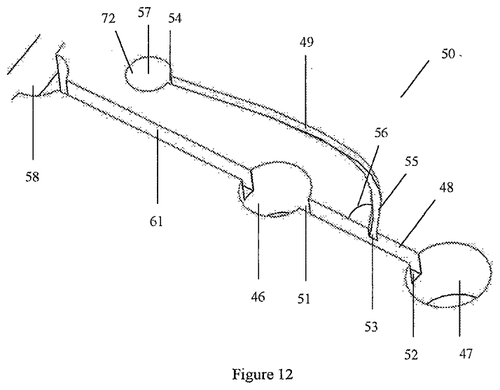

The structure of the centrifugal microfluidic chip for nucleic acid multiplex detection provided in this example is shown in FIGS. 9-12, where FIG. 9 is a front view of the centrifugal microfluidic chip (first type of exhaust channel), FIG. 10 is a back view of the centrifugal microfluidic chip (first type of exhaust channel); FIG. 11 is a perspective view of the centrifugal microfluidic chip (first type of exhaust channel); FIG. 12 is a schematic structural diagram of the detection unit in the centrifugal microfluidic chip (first type of exhaust channel).

As shown in FIGS. 9-11, the centrifugal microfluidic chip 43 for nucleic acid multiplex detection provided in this example includes a chip body 45 with a rotation center 44 and a sub-chip 69 arranged outside the chip body 45. Multiple groups of detection units 8 can be arranged on the chip body 45 and the sub-chip 69 according to project needs or the size of the chip to realize the simultaneous detection of multiple targets. Each group of detection units 50 is provided with a reaction chamber 46 and a detection chamber 47. The reaction chamber 46 is used for nucleic acid amplification of the sample, and the detection chamber 47 is used for sample detection. Therefore, each group of detection units can realize nucleic acid multiplex detection. When the chip body 45 rotates centrifugally, the sub-chip 69 can rotate centrifugally along with the chip body 45. Both the chip body 45 and the sub-chip 69 are provided with a main flow channel 58, and the main flow channel 58 is also provided with a sample-adding port 59 (including a first sample-adding port 171 in the chip body 45 and a second sample-adding port 172 in the sub-chip 27). The main flow channel 58 is a connected circular closed loop, and the sample-adding port 59 is an opening set in the circular closed loop of the main flow channel 58 for sample injection. The main flow channel 58 is arranged in a wave shape and bent into a circular ring. The chip body 45, the circular ring, and the rotation center 44 are coaxially arranged. The reaction chamber 46 and the detection chamber 47 are connected through a fluid channel 48, and the fluid channel 48 is further provided with an exhaust channel 49 led out from approximately the middle of the fluid channel 48. The exhaust channel 49 is used for controlling the flow of gas in the reaction chamber 46 or the detection chamber 47; the rotation center 44 is used for connecting with a centrifugal driving device to drive the centrifugal microfluidic chip 43 to centrifuge.

During the use of the centrifugal microfluidic chip 43, it needs to be placed horizontally in a matching detection device (centrifugal driving device) with the sample-adding port 51 facing upward. The rotation center 44 is buckled and fixed with the rotating shaft for centrifugation of the detection device, so that the rotating shaft can drive the centrifugal microfluidic chip 43 to perform centrifugal rotation; at the same time, the detection device can provide a heat source to heat the reaction chamber 46 of the centrifugal microfluidic chip 43; it can also detect the fluorescent substances generated after the sample reaction in the centrifugal microfluidic chip 43 through the detection device and read the detection results.

The chip body 45 is circular, and the sub-chip 69 is located on a concentric circle outside the chip body 45. The chip body 45 and the sub-chip 69 can be directly molded and manufactured in one piece and cannot be disassembled. Of course, if necessary, sub-chips can be further arranged outside the sub-chip 69. The sub-chip 69, the chip body 45, and the entire microfluidic chip 43 are all in the same plane. The sub-chip 69 is located outside the chip body 45 and has a larger diameter, so more groups of detection units 50 can be arranged. The sub-chip 69 can also independently perform nucleic acid multiplex detection, detect samples different from those of the chip body 45, realize the simultaneous detection of multiple samples, and detect more indicators at the same time (each detection unit can detect one nucleic acid target), significantly increasing the detection throughput and improving the detection efficiency. The number of sub-chips 69 and the number of detection units 50 in each sub-chip 69 or chip body 45 can be flexibly set according to the number of samples to be detected and the requirements of indicator detection. The solid reagents pre-embedded in the reaction chamber 46 and the detection chamber 47 of each detection unit 50 can be flexibly set according to actual needs. For example, different amplification primers for respiratory targets are pre-embedded in different reaction chambers 46, and corresponding detection reagents are pre-embedded in the corresponding detection chambers 47, so that multiplex detection of respiratory pathogens can be flexibly realized.

As shown in FIGS. 9-12, an exhaust channel 49 is arranged on the fluid channel 48 connecting the reaction chamber 46 and the detection chamber 47 in each detection unit 50 of the centrifugal microfluidic chip 43, so that the reaction chamber 46 and the detection chamber 47 share one exhaust channel 49. The fluid channel 48 is a channel connecting the outlet 51 of the reaction chamber 46 and the inlet 52 of the detection chamber 47. The exhaust channel 49 includes an inlet 53 and an outlet 54. The inlet 53 is the connection between the fluid channel 48 and the exhaust channel 49, and the outlet 54 is the other end of the exhaust channel 49; the outlet 54 is closer to the rotation center 44 than the inlet 53, so that the exhaust direction of the exhaust channel 49 is opposite or at least partially opposite to the rotational centrifugal direction. When the centrifugal microfluidic chip 43 is centrifuged, the flow direction of the fluid sample under the action of centrifugation is from the center of the circle to the outside. At this time, since the exhaust channel 49 is led out from the fluid channel 48 between the reaction chamber 46 and the detection chamber 47 and then approaches the rotation center 44, the exhaust direction is opposite to the centrifugal direction. The fluid sample will not enter the exhaust channel 49 during centrifugation, but accurately enter the corresponding chamber along the centrifugal direction.

Preferably, as shown in FIG. 12, when the centrifugal microfluidic chip 43 is placed horizontally, the outlet 54 of the exhaust channel 49 is higher than the inlet 53. This design can further increase the difficulty of the fluid sample entering the exhaust channel 49, thereby helping to control the flow of liquid in the chip, preventing the sample from entering the exhaust channel 49, and at the same time facilitating the discharge of gas from the reaction chamber 46 and the detection chamber 47, so that the centrifugal microfluidic chip 43 can perform nucleic acid multiplex detection more accurately.

As shown in FIG. 12, the exhaust channel 49 is led out from the middle of the fluid channel or a position close to the detection chamber 47, and the initial section of the lead-out is an arc-shaped channel 55. The angle 56 between the arc-shaped channel 55 and the fluid channel close to the reaction chamber 46 is less than 90 degrees. The exhaust channel 49 can be led out slightly close to the detection chamber 47. Because if the exhaust channel 49 is led out close to the detection chamber, the pressure that the sample needs to overcome to enter the detection chamber 47 during centrifugation can be reduced, and the sample can more easily enter the detection chamber 47 during centrifugation. When the exhaust channel 49 is led out from the fluid channel 48, the smaller the angle 56 between the initial section and the fluid channel close to the reaction chamber 46, the more difficult it is for the fluid to enter the exhaust channel 49. However, this angle 56 cannot be too small, otherwise it may cause difficulty in exhaust. It is preferable to use an arc-shaped channel 55 at one end for transition, and the angle between the arc-shaped channel 55 and the fluid channel close to the reaction chamber 46 is less than 90 degrees. Preferably, the inner diameter of the exhaust channel 49 is smaller than that of the fluid channel 48, which can further prevent the sample from entering the exhaust channel 49 during centrifugation.

As shown in FIG. 12, the outlet 54 of the exhaust channel 49 is connected to the outside, and the outlet 54 connected to the outside is covered with a waterproof and breathable membrane 57. The waterproof and breathable membrane 57 is a membrane that can allow air to pass through but not water, which can realize the discharge of gas from the reaction chamber 46 or the detection chamber 47, but the liquid cannot pass through. The waterproof and breathable membrane 57 can help the sample in the reaction chamber 46 or the detection chamber 47 establish an internal and external pressure balance, and can regulate the flow direction of the liquid, so that the sample in the reaction chamber 46 or the detection chamber 47 will not leak or flow back. The waterproof and breathable membrane 57 can be made of a polymer material, such as high-grade synthetic rubber, which has good sealing performance, no back glue and residue, and does not react with reaction reagents or detection reagents. In this example, the waterproof and breathable membrane is preferably made of a material (manufacturer: Membrane; article number: VET022H60). When this setting mode is adopted, nucleic acid multiplex detection can be completed with only one centrifugation in the whole process of the centrifugal microfluidic chip 43. Preferably, the outlet 54 of the exhaust channel 49 is further provided with a gas-permeable chamber 72, and the volume of the gas-permeable chamber 72 is not less than that of the reaction chamber 46, which is used to help the reaction chamber 46 exhaust and make it easier for the sample to enter the reaction chamber 46.

The flow process of the sample to be tested in the centrifugal microfluidic chip 43 mainly includes two steps: the first step, the sample to be tested enters the reaction chamber 46 through the main flow channel 58 under the action of injection pressure for reaction; the second step, after the reaction is completed, the sample to be tested flows out of the reaction chamber 46 through low-speed centrifugation and enters the detection chamber 47 for reaction and detection.

The sample enters the wave-shaped main flow channel 58 from the sample-adding port 59 through a pipette or syringe injection, and flows into each reaction chamber 46 from the main flow channel 58 under the action of injection pressure, then the sample-adding port 59 is sealed. In the first step, during the process of the sample entering the reaction chamber 46, the gas in the reaction chamber 46 needs to be discharged to the outside through the exhaust channel 49. Since the detection chamber 47 is not provided with an exhaust channel 49, the sample is difficult to flow into it. At the same time, because the exhaust direction of the exhaust channel 49 is opposite to the centrifugal direction and higher than the fluid channel 48, and the inner diameter of the exhaust channel 49 is smaller than that of the fluid channel 48, the sample can only reach the intersection of the fluid channel 48 and the exhaust channel 49 (the inlet 53 of the exhaust channel 49) at most in the first step, and cannot continue to move forward, thus controlling the volume of the sample entering the reaction chamber 46. At this time, since the sample-adding port 59 is sealed, that is, only the exhaust channel 49 of the entire centrifugal microfluidic chip 43 is connected to the atmosphere, and the other parts are in a sealed state, the internal and external pressure balance is achieved. The sample in the reaction chamber 46 can no longer flow from the reaction chamber 46 to other areas, because flowing out of the reaction chamber 46 will inevitably require a large pressure to break the established internal and external pressure balance. Therefore, all the samples will remain in the reaction chamber 46 for reaction, and no leakage or backflow will occur. In the second step, after the sample completes the reaction in the reaction chamber 46, through low-speed centrifugation (such as 100-1000 rpm, preferably 200-800 rpm in this example, more preferably 500-600 rpm), the initial pressure balance is broken, and the sample flows out of the reaction chamber 46 under the action of centrifugal force. At this time, because the exhaust direction of the exhaust channel 49 is opposite to the centrifugal direction, the sample will not enter the exhaust channel 49, but will all flow to the detection chamber 47, and the gas in the detection chamber 47 is discharged from the exhaust channel 49. At this time, the pressure balance is also achieved in the detection chamber 47, so that the sample in the detection chamber 47 can no longer flow out of the detection chamber 47, and all remain in the detection chamber 47 for reaction and detection.

Therefore, when the first setting mode is adopted, under the combined action of the exhaust channel 49 and the fluid channel 48, the sample in the reaction chamber 46 or the detection chamber 47 can establish an internal and external pressure balance, the flow direction of the liquid can be regulated, the sample in the reaction chamber 46 or the detection chamber 47 will not leak or flow back, and the amount of sample entering the reaction chamber 46 or the detection chamber 47 can be effectively controlled. Nucleic acid multiplex detection can be completed with only one low-speed centrifugation.

Preferably, the main flow channel 58 adopts a wave-shaped flow channel with a large curvature and uniformly spaced arrangement, which can help maintain the pressure balance inside the chip 43, stably control the flow of liquid, and help prevent direct mutual interference between each detection unit 50.

The number of sub-chips 69 and the number of reaction chambers 46 and detection chambers 47 arranged on each sub-chip 69 and the pre-embedded solid reagents can be flexibly set according to needs. Preferably, the number of sub-chips 69 can be set to 2-5, and the number of amplification chambers 46 and detection chambers 47 can be set to 4-80. In this example, the number of sub-chips 69 is 1, the chip body 45 is provided with 34 reaction chambers 46 and 34 detection chambers 47, and the sub-chip 69 is provided with 66 reaction chambers 46 and 66 detection chambers 47.

The surface of the sample-adding port 59 of the centrifugal microfluidic chip 43 is covered with a transparent film for sealing. During sample addition, a syringe is used to pierce the transparent film for sample addition. After the main flow channel 58 is filled with the sample, the surface of the sample-adding port 59 needs to be covered with a transparent film again for sealing.

As shown in FIGS. 9-12, the rotation center 44 is a clamping groove 70, which can be clamped on the rotating shaft of the detection device for centrifugal rotation, thereby driving the entire chip to rotate centrifugally. Preferably, the clamping groove 70 is a circular clamping groove with protrusions 71, and the shape of the protrusions 71 is triangular, trapezoidal, etc. The rotating shaft on the detection device is also provided with a fixing groove matching the size of the clamping groove 70, so that the centrifugal microfluidic chip 43 is fixed on the rotating shaft through the clamping groove 70 and rotates centrifugally along with the rotating shaft.

Preferably, the cross-section of the reaction chamber 46 is circular, so that the sample to be tested can react in the reaction chamber 46 and be discharged without leaving dead corners, and it is not easy to remain during the inflow and outflow processes. The cross-section of the detection chamber 5 is approximately semicircular and larger than that of the reaction chamber 46, which can ensure that all samples in the reaction chamber 46 enter the detection chamber 47.

In some embodiments, the reaction chamber 46 is used for amplifying the target nucleic acid, nucleic acid amplification reagents are pre-embedded in the reaction chamber 46, the detection chamber 47 is used for detecting the target nucleic acid, and nucleic acid detection reagents are pre-embedded in the detection chamber 47; in some embodiments, the reaction chamber 46 can be used as an ERA amplification chamber, and the detection chamber 47 can be used as an ERA detection chamber; in some embodiments, the reaction chamber 46 can be used as an ERA reaction chamber, and the detection chamber 47 can be used as a CRISPR detection chamber; in some embodiments, the reaction chamber 46 can be used as an ERA reaction chamber, and the detection chamber 47 can be used as an Ago detection chamber; in some embodiments, the reaction chamber 46 can be used as an ERA reaction chamber, and the detection chamber 47 can be used as an RNase H detection chamber; in some embodiments, the amplification reaction includes, but is not limited to, EPA, RPA, LAMP, NEAR, NASBA, HAD, TMA, SAT, RCR, etc., and the form of the solid amplification reagent includes, but is not limited to, spherical, powdery, flaky, or blocky preparations in the form of freeze-drying, drying, air-drying, etc. In some embodiments, the detection reagents include, but are not limited to, various detection systems established based on ERA, RPA, LAMP, NEAR, NASBA, HAD, TMA, SAT, PCR, hybridization probe technology, CRISPR, Ago, RNase H, etc. The detection reagents are solid detection reagents, including but not limited to spherical, powdery, flaky, or blocky preparations in the form of freeze-drying, drying, air-drying, etc.

When the sample to be tested is added to the sample-adding port 59 through a pipetting device (not shown in the figure), the sample to be tested can be evenly divided into branch channels 61 along the main flow channel 58, reach the reaction chamber 46, mix with the solid amplification reagents therein, and undergo an amplification reaction at a certain temperature; when the amplification reaction is completed, the rotating shaft in the detection device is driven to rotate clockwise, driving the centrifugal microfluidic chip 43 to perform centrifugal movement. The liquid in the reaction chamber 46 is transferred to the detection chamber 47, and a fluorescent detection reaction occurs. After the experiment is completed, the result can be observed with the naked eye through the perspective window of the detection device, or an RGB sensor can be set in the detection device for automatic identification and result judgment. Preferably, an LED lamp or a laser lamp is set in the detection device, and blue light is emitted to irradiate the detection chamber 47, thereby improving the color development effect of the detection chamber 47 (when observed with the naked eye as green, it is judged that positive amplification has occurred; when observed as dark orange, it is judged as negative). The above are all existing technologies and will not be repeated.

Preferably, the main flow channel 58, the branch channels 61 (including the fluid channel 48), and the gas-permeable channel 49 all adopt a gradually narrowing micro-channel structure to prevent uneven sample distribution caused by too fast sample droplets; the width of the main flow channel 58 ranges from 500 μm to 1000 μm, and the depth ranges from 500 μm to 1000 μm; the width of the branch channels 61 ranges from 400 μm to 800 μm, and the depth ranges from 150 μm to 300 μm; the width of the gas-permeable channel 49 is 50 μm to 500 μm, and the depth is 50-350 μm. In this example, the widths of the main flow channel 58, the branch channels 61, and the gas-permeable channel 49 are 800 μm, 500 μm, and 300 μm respectively, and the depths are 500 μm, 200 μm, and 100 μm respectively.

The centrifugal microfluidic chip 43 provided in this example uses materials with low cost, easy processing performance, and good biocompatibility for chips, such as polymethyl methacrylate (PMMA), polycarbonate (PC), polypropylene (PP), etc. The waterproof and breathable membrane is attached to the chip body to form a sealed space, effectively preventing liquid leakage. According to detection needs, the reaction chamber 46 is filled with fixed amplification reagents for nucleic acid amplification, and the detection chamber 47 is filled with solid detection substances for detection, such as freeze-dried balls, freeze-dried powders, or quality control internal standard reagents.

The centrifugal microfluidic chip 43 is designed with multiple chips (the chip body 45 and the sub-chip 69). Each chip operates independently and is combined with an active driving method. While accurately controlling the reconstitution and mixing processes of samples and reagents, it has the characteristics of ultra-high throughput and can be used for multi-indicator detection of multiple samples (such as the field of screening multiple different respiratory pathogens in samples of multiple patients).

### Example 5: Centrifugal Microfluidic Chip with the First Type of Exhaust Channel Setting (No Sub-Chip)

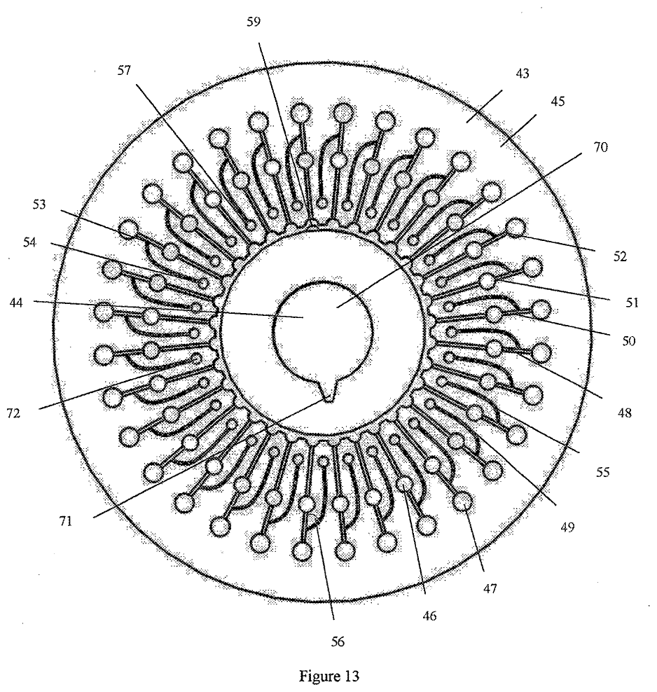

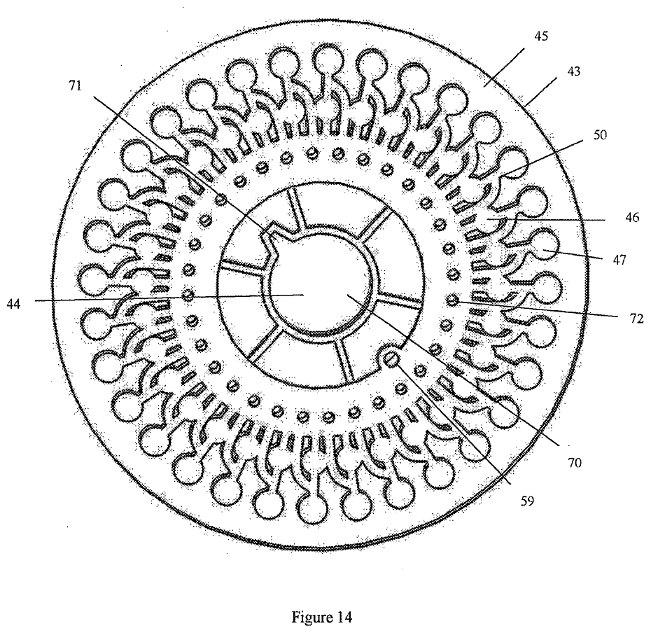

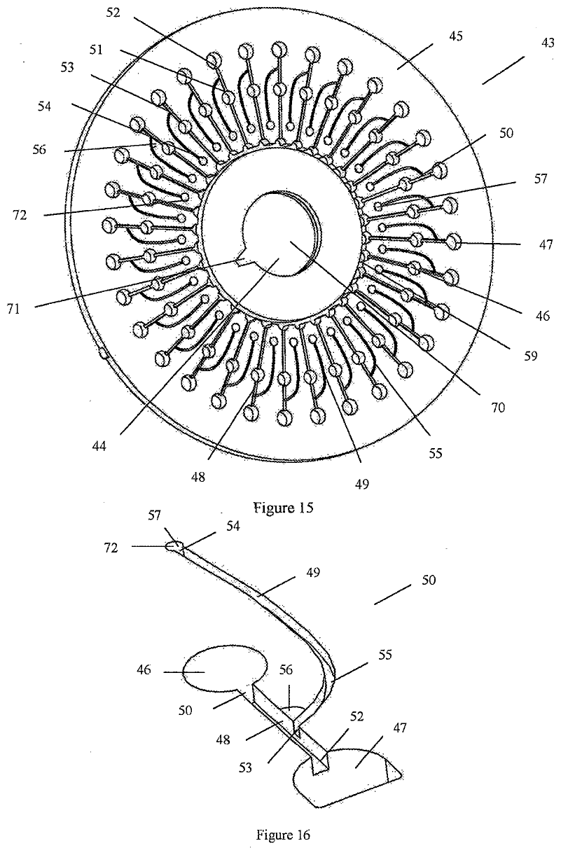

The structure of the centrifugal microfluidic chip for nucleic acid multiplex detection provided in this example is shown in FIGS. 13-16, where FIG. 13 is a front view of the centrifugal microfluidic chip (first type of exhaust channel), FIG. 14 is a back view of the centrifugal microfluidic chip (first type of exhaust channel); FIG. 15 is a perspective view of the centrifugal microfluidic chip (first type of exhaust channel); FIG. 16 is a schematic structural diagram of the detection unit in the centrifugal microfluidic chip (first type of exhaust channel).

As shown in FIGS. 13-16, the centrifugal microfluidic chip 43 for nucleic acid multiplex detection provided in this example is different from the chip in Example 4 in that it only includes the chip body 45 and no sub-chip 69.

The centrifugal microfluidic chip 43 provided in this example is combined with a centrifugal driving method. While accurately controlling the reconstitution and mixing processes of samples and reagents, it has the characteristics of ultra-high throughput and can be used for multi-indicator detection of a single sample (such as the field of screening multiple different respiratory pathogens in a single patient sample).

### Example 6

(1) Centrifugal Microfluidic Chip for Nucleic Acid Multiplex Detection with the Second Type of Exhaust Channel Setting (Corresponding to FIGS. 17-20)

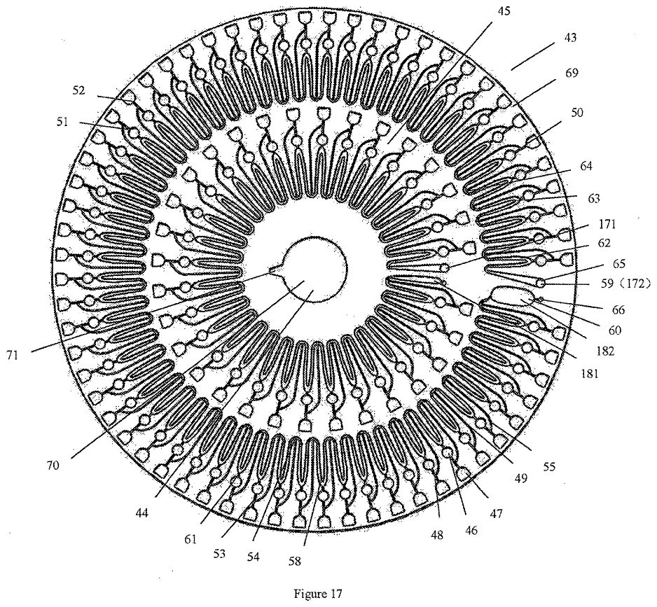

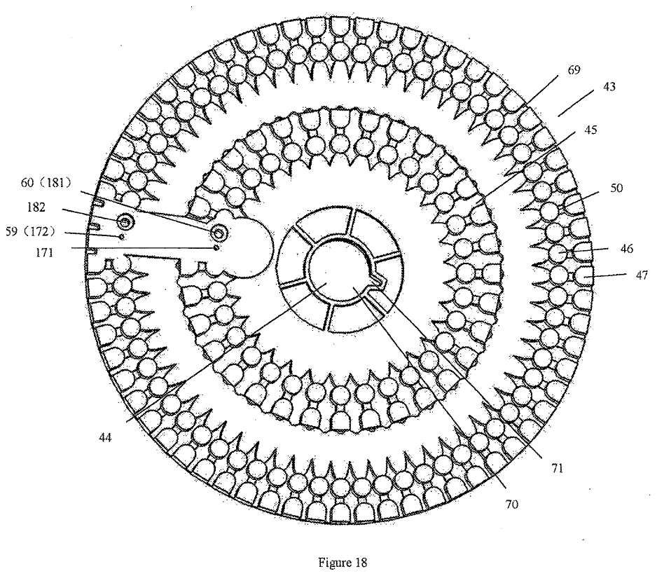

The structure of the centrifugal microfluidic chip for nucleic acid multiplex detection provided in this example is shown in FIGS. 17-20, where FIG. 17 is a front view of the centrifugal microfluidic chip (second type of exhaust channel); FIG. 18 is a back view of the centrifugal microfluidic chip (second type of exhaust channel); FIG. 19 is a perspective view of the centrifugal microfluidic chip (second type of exhaust channel); FIG. 20 is a schematic structural diagram of the detection unit in the centrifugal microfluidic chip (second type of exhaust channel).

As shown in FIGS. 17-20, the centrifugal microfluidic chip 43 for nucleic acid multiplex detection provided in this example includes a chip body 45 with a rotation center 44. The chip body 45 is provided with a reaction chamber 46 for nucleic acid amplification of the sample and a detection chamber 47 for sample detection; the reaction chamber 46 and the detection chamber 47 are connected through a fluid channel 48, and the fluid channel 48 is further provided with an exhaust channel 49 led out from approximately the middle of the fluid channel 48. The exhaust channel 49 is used for controlling the flow of gas in the reaction chamber 46 or the detection chamber 47; the rotation center 44 is used for connecting with a centrifugal driving device to drive the centrifugal microfluidic chip 43 to centrifuge.

Any number of groups of detection units 50 can be arranged on the centrifugal microfluidic chip 43 according to project needs or the size of the chip to realize the simultaneous detection of multiple targets. Each group of detection units 50 is provided with a reaction chamber 46 and a detection chamber 47, which can be applied to the detection of multiple different target analytes (such as multiple different nucleic acid targets), and each group of detection units can realize nucleic acid multiplex detection. During the use of the centrifugal microfluidic chip 43, it needs to be placed horizontally in a matching detection device (centrifugal driving device) with the sample-adding port 51 facing upward. The rotation center 44 is buckled and fixed with the rotating shaft for centrifugation of the detection device, so that the rotating shaft can drive the centrifugal microfluidic chip 43 to perform centrifugal rotation; at the same time, the detection device can provide a heat source to heat the reaction chamber 46 of the centrifugal microfluidic chip 43; it can also detect the fluorescent substances generated after the sample reaction in the centrifugal microfluidic chip 43 through the detection device and read the detection results.

The fluid channel 48 connecting the reaction chamber 46 and the detection chamber 47 is a channel connecting the outlet 51 of the reaction chamber 46 and the inlet 52 of the detection chamber 47. The exhaust channel 49 includes an inlet 53 and an outlet 54. The inlet 53 is the connection between the fluid channel 48 and the exhaust channel 49, and the outlet 54 is the other end of the exhaust channel 49; the outlet 54 is closer to the rotation center 44 than the inlet 53, so that the exhaust direction of the exhaust channel 49 is opposite or at least partially opposite to the rotational centrifugal direction. When the centrifugal microfluidic chip 43 is centrifuged, the flow direction of the fluid sample under the action of centrifugation is from the center of the circle to the outside. At this time, since the exhaust channel 49 is led out from the fluid channel 48 between the reaction chamber 46 and the detection chamber 47 and then approaches the rotation center 44, the exhaust direction is opposite to the centrifugal direction. The fluid sample will not enter the exhaust channel 49 during centrifugation, but accurately enter the corresponding chamber along the centrifugal direction.

As shown in FIG. 20, the exhaust channel 49 is led out from the middle of the fluid channel or a position close to the detection chamber 47, and the initial section of the lead-out is an arc-shaped channel 55. The angle 56 between the arc-shaped channel 55 and the fluid channel close to the reaction chamber 46 is less than 90 degrees. The exhaust channel 49 can be led out slightly close to the detection chamber 47. Because if the exhaust channel 49 is led out close to the detection chamber, the pressure that the sample needs to overcome to enter the detection chamber 47 during centrifugation can be reduced, and the sample can more easily enter the detection chamber 47 during centrifugation. When the exhaust channel 49 is led out from the fluid channel 48, the smaller the angle 56 between the initial section and the fluid channel close to the reaction chamber 46, the more difficult it is for the fluid to enter the exhaust channel 48. However, this angle 56 cannot be too small, otherwise it may cause difficulty in exhaust. It is preferable to use an arc-shaped channel 55 at one end for transition, and the angle between the arc-shaped channel 55 and the fluid channel close to the reaction chamber 46 is less than 90 degrees. Preferably, the inner diameter of the exhaust channel 49 is smaller than that of the fluid channel 48 (as shown in FIG. 20), which can further prevent the sample from entering the exhaust channel 49 during centrifugation.

The outlet 54 of the exhaust channel 49 is in communication with the main flow channel 58 of the chip body 45 and exhausts to the main flow channel 58 (see FIGS. 9-12). When this setting mode is adopted, nucleic acid multiplex detection can be completed with only two centrifuging steps in the whole process. Neither the reaction chamber 46 nor the detection chamber 47 needs to be connected to the external atmosphere. The exhaust channel 49 is directly connected to the main flow channel 58, and the atmosphere pressed out of the reaction chamber 46 or the detection chamber 47 is discharged back to the main flow channel 58, which is equivalent to discharging the air pressure in the liquid-filled part to the unfilled part through the exhaust channel 49, playing a role in controlling the flow of liquid. Moreover, after exhausting to the main flow channel 58 through the exhaust channel 49, the pressure balance is achieved inside the chip 43, and the sample entering the reaction chamber 46 or the detection chamber 47 is also difficult to flow out of the reaction chamber 46, and backflow is difficult to occur, forming a circular closed environment. Compared with the setting mode in Example 4 or 5 where the outlet 54 of the exhaust channel is connected to the outside, since it is not connected to the outside, aerosol contamination can be effectively avoided, and the detection result is more accurate and reliable. At the same time, since the steps of preparing air vents and covering waterproof and breathable membranes 57 are omitted, the preparation process is simpler and easier, and the cost is lower.

When this setting mode is adopted, the flow and detection process of the sample to be tested in the centrifugal microfluidic chip 43 includes three steps: the first step, the sample enters the main flow channel 58 from the sample-adding port 59; the second step, low-speed centrifugation is performed to make the sample enter the reaction chamber 46 from the main flow channel 58 for reaction; the third step, after the reaction is completed, the sample flows out of the reaction chamber 46 through medium-speed centrifugation and enters the detection chamber 47 for reaction and detection. Different from the first setting mode, when the second setting mode is adopted, it is also necessary to control the flow of the sample by controlling the centrifugal speed. The sample enters the reaction chamber 5 from the main flow channel 16 through low-speed centrifugation (such as 100-1000 rpm), and then enters the detection chamber 47 from the reaction chamber 46 through medium-speed centrifugation (such as 1000-3000 rpm). The method of controlling the flow of the sample in different chambers by controlling the centrifugal speed is simpler and easier to operate, and the whole process is more controllable, and the detection result is more accurate.

As shown in FIGS. 17-20, when the first step is adopted in this setting mode, the sample enters the main flow channel 58 from the sample-adding port 59 through a pipette or syringe injection. After filling the serpentine main flow channel 58, the sample will flow to the final waste liquid pool 60, then the injection is stopped, and the sample-adding port 59 is sealed. This process is equivalent to the process of filling the main flow channel 58 with the sample, and also the process of discharging the gas after being filled with the liquid to the unfilled part of the main flow channel 58 through the exhaust channel 7. Since the inner diameter of the branch channel 61 connecting the main flow channel 58 and the reaction chamber 46 is small, and there is a large difference between the inner diameter of the main flow channel 58 and the branch channel 61, the sample cannot flow into the reaction chamber 46. Moreover, it is difficult to discharge the gas in the reaction chamber 46 back to the main flow channel 58 from the exhaust channel 49 only by the injection pressure, so the sample will stay in the main flow channel 58 first. In the second step, after the main flow channel 58 is completely filled with the sample, clockwise low-speed instantaneous centrifugation is performed (such as 100-1000 rpm, preferably 500 rpm in this example). The sample in the main flow channel 58 enters the reaction chamber 46 under the action of centrifugal force. Since the detection chamber 47 is not provided with a separate exhaust channel 49, it is almost in an enclosed structure. Under low-speed centrifugation, the sample is difficult to overcome the gas pressure in the detection chamber 47, so it cannot flow into the detection chamber 47. At the same time, because the exhaust direction of the exhaust channel 49 is opposite to the centrifugal direction and the exhaust channel 49 is higher than the fluid channel 48, the sample can only reach the intersection of the fluid channel 48 and the exhaust channel 49 at most in the second step, and cannot continue to move forward, thus controlling the volume of the sample entering the reaction chamber 46. During the low-speed centrifugation, the sample enters the reaction chamber 46, and at the same time, the gas in the reaction chamber 46 is discharged back to the main flow channel 58, which can also help the sample in the main flow channel 58 flow into the reaction chamber 46. After low-speed centrifugation, the sample remains in the reaction chamber 46 for amplification reaction. In a sealed state, the internal and external pressure balance is achieved. The sample in the reaction chamber 46 can no longer flow from the reaction chamber 46 to other areas, because flowing out of the reaction chamber 46 will inevitably require a large pressure to break the established internal and external pressure balance. Therefore, all the samples will remain in the reaction chamber 46 for reaction, and no leakage or backflow will occur. In the third step, after the sample completes the amplification reaction in the reaction chamber 46, the centrifugal speed is increased, and medium-speed centrifugation is performed (such as 1000-3000 rpm, preferably 2000 rpm in this example). The initial pressure balance is broken, and the sample is driven to transfer from the reaction chamber 46 to the detection chamber 47. At this time, because the exhaust direction of the exhaust channel 49 is opposite to the centrifugal direction and the inner diameter of the exhaust channel 49 is smaller, the sample will not enter the exhaust channel 49, but will all flow to the detection chamber 47. Since the entire system is in a sealed state, the liquid in the detection chamber 47 will not flow back, thus being stably used for detection.

Therefore, the second setting mode adopts two-stage centrifugation to complete nucleic acid multiplex detection. The first-stage centrifugation speed and the second-stage centrifugation speed can be quite different, and the control is stable. Moreover, high-speed centrifugation (such as 3000-10000 rpm) is not required, which avoids the precipitation of pre-embedded freeze-dried or vitrified reagents due to insufficient dissolution caused by high-speed centrifugation, thereby causing reaction failure and affecting the detection results. In addition, it is not connected to the outside, so aerosol contamination is avoided, and the manufacturing is more convenient and the cost is lower.

The chip body 45 is circular, and the main flow channel 58 is arranged in a serpentine shape and bent into a circular ring. The chip body 45, the circular ring, and the rotation center 44 are coaxially arranged. The main flow channel 58 adopts a serpentine flow channel with a large curvature and uniformly spaced arrangement, which can help maintain the pressure balance inside the chip 43, stably control the flow of liquid, and help prevent direct mutual interference between each detection unit 50.

As shown in FIGS. 17-20, when the outlet 54 of the exhaust channel 49 is in communication with the main flow channel 58, the main flow channel 58 is arranged in a serpentine shape. The serpentine arrangement makes the main flow channel 58 have a larger bending degree and a longer length, so that the sample can stay stably in the main flow channel 58 first. The serpentine-arranged main flow channel 58 includes U-shaped flow channels 62 arranged at equal distances along the circular ring. The bottom end 63 of the U-shaped flow channel 62 is connected to the reaction chamber 46; when the outlet 54 of the exhaust channel 49 is in communication with the main flow channel 58, the communication part is the top end 64 of the U-shaped flow channel 62.

Preferably, the bottom end 63 of the U-shaped flow channel 62 is provided with a branch channel 61 connected to the inlet of the reaction chamber, the top end 64 of the U-shaped flow channel 62 is connected to the exhaust channel 49, the left and right sides of the U-shaped flow channel 62 are longer, and the top end 64 is closer to the rotation center 44 than the bottom end 63. As a result, the distance between the bottom end 63 and the top end 64 of the U-shaped flow channel 62 is relatively far. When the exhaust channel 49 exhausts to the top end 64 of the U-shaped flow channel 62, it will not immediately affect the pressure balance at the bottom end 63, which helps the sample flow into the reaction chamber 46 smoothly. As shown in FIGS. 17-19, the U-shaped flow channels 62 are arranged uniformly, and the bottom end 63 of each U-shaped flow channel 62 is connected to a reaction chamber 46. Therefore, during centrifugation, the sample in each U-shaped flow channel 62 will enter the corresponding reaction chamber 46 under the action of centrifugal force, which is equivalent to the U-shaped flow channel 62 pre-distributing the sample uniformly, and can assist in controlling the volume of the sample entering the reaction chamber 46.

Preferably, the main flow channel 58, the branch channels 61 (including the fluid channel 48), and the gas-permeable channel 49 all adopt a gradually narrowing micro-channel structure to prevent uneven sample distribution caused by too fast sample droplets; the width of the main flow channel 58 ranges from 500 μm to 1000 μm, and the depth ranges from 500 μm to 1000 μm; the width of the branch channels 61 ranges from 400 μm to 800 μm, and the depth ranges from 200 μm to 400 μm; the width of the gas-permeable channel 49 is 50 μm to 500 μm, and the depth is 100-300 μm. In this example, the widths of the main flow channel 58, the branch channels 61, and the gas-permeable channel 49 are 800 μm, 400 μm, and 200 μm respectively, and the depths are 500 μm, 400 μm, and 200 μm respectively.

The main flow channel 58 is provided with a sample-adding port 59 (including a first sample-adding port 171 in the chip body 45 and a second sample-adding port 172 in the sub-chip 69). As shown in FIGS. 17-20, the sample-adding port 59 is located at the starting end 65 of the main flow channel 58, and the end 66 of the main flow channel 58 is further provided with a waste liquid pool 60 (including a first waste liquid pool 181 in the chip body 45 and a second waste liquid pool 182 in the sub-chip 69). The two ends of the main flow channel 58 are not connected. The waste liquid pool 60 has a liquid storage function and is used for receiving excess samples. During the process of filling the main flow channel 58 with the sample, the excess sample will enter the waste liquid pool 60. Since the injection pressure is not sufficient to make the sample enter the reaction chamber 46, after the main flow channel 58 is filled with the sample, the excess sample will start to enter the waste liquid pool 60. At this time, it also indicates that the sample volume is sufficient, and the sample addition can be stopped. Therefore, the waste liquid pool 60 also has the effect of helping to quantify the sample.

The surface of the sample-adding port 59 of the centrifugal microfluidic chip 43 is covered with a transparent film for sealing. During sample addition, a syringe is used to pierce the transparent film for sample addition. After the main flow channel 58 is filled with the sample, the surface of the sample-adding port 59 needs to be covered with a transparent film again for sealing.

As shown in the centrifugal microfluidic chip 43 provided in FIGS. 17-20, a sub-chip 69 is further arranged outside the chip body 45. The sub-chip 69 is a concentric circular ring chip located outside the chip body 45. The sub-chip 69 is also provided with a main flow channel 58, a reaction chamber 46, and a detection chamber 47; when the chip body 45 rotates centrifugally, the sub-chip 69 can rotate centrifugally along with the chip body 45. The sub-chip 69 is a concentric circular ring chip located outside the chip body 45. Of course, if necessary, sub-chips can be further arranged outside the sub-chip 69. The chip body 45 and the sub-chip 69 can be directly molded and manufactured in one chip 43 and cannot be disassembled. The sub-chip 69, the chip body 45, and the entire microfluidic chip 43 are all in the same plane. The sub-chip 69 is located outside the chip body 45 and has a larger diameter, so more groups of detection units 50 can be arranged. At the same time, the sub-chip 69 can also be provided with a sample-adding port 59 and a waste liquid pool 60 to independently perform nucleic acid multiplex detection, detect samples different from those of the chip body 45, realize the simultaneous detection of multiple samples, and detect more indicators at the same time (each detection unit 50 can detect one nucleic acid target), significantly increasing the detection throughput and improving the detection efficiency.

As shown in FIGS. 17-20, the rotation center 44 is a clamping groove 70, which can be clamped on the rotating shaft of the detection device for centrifugal rotation, thereby driving the entire chip to rotate centrifugally. Preferably, the clamping groove 70 is a circular clamping groove with protrusions 71, and the shape of the protrusions 71 is triangular, trapezoidal, etc. The rotating shaft on the detection device is also provided with a fixing groove matching the size of the clamping groove 70, so that the centrifugal microfluidic chip 43 is fixed on the rotating shaft through the clamping groove 70 and rotates centrifugally along with the rotating shaft.

Preferably, the cross-section of the reaction chamber 46 is circular, so that the sample to be tested can react in the reaction chamber 46 and be discharged without leaving dead corners, and it is not easy to remain during the inflow and outflow processes. The cross-section of the detection chamber 47 is approximately semicircular and larger than that of the reaction chamber 46, which can ensure that all samples in the reaction chamber 46 enter the detection chamber 47.

In some embodiments, the reaction chamber 46 is used for amplifying the target nucleic acid, nucleic acid amplification reagents are pre-embedded in the reaction chamber 46, the detection chamber 47 is used for detecting the target nucleic acid, and nucleic acid detection reagents are pre-embedded in the detection chamber 47.

The centrifugal microfluidic chip 43 provided in this example is designed with multiple chips (the chip body 35 and the sub-chip 69). Each chip operates independently and is combined with an active driving method. While accurately controlling the reconstitution and mixing processes of samples and reagents, it has the characteristics of ultra-high throughput and can be used for multi-indicator detection of multiple samples (such as the field of screening multiple different respiratory pathogens in samples of multiple patients).

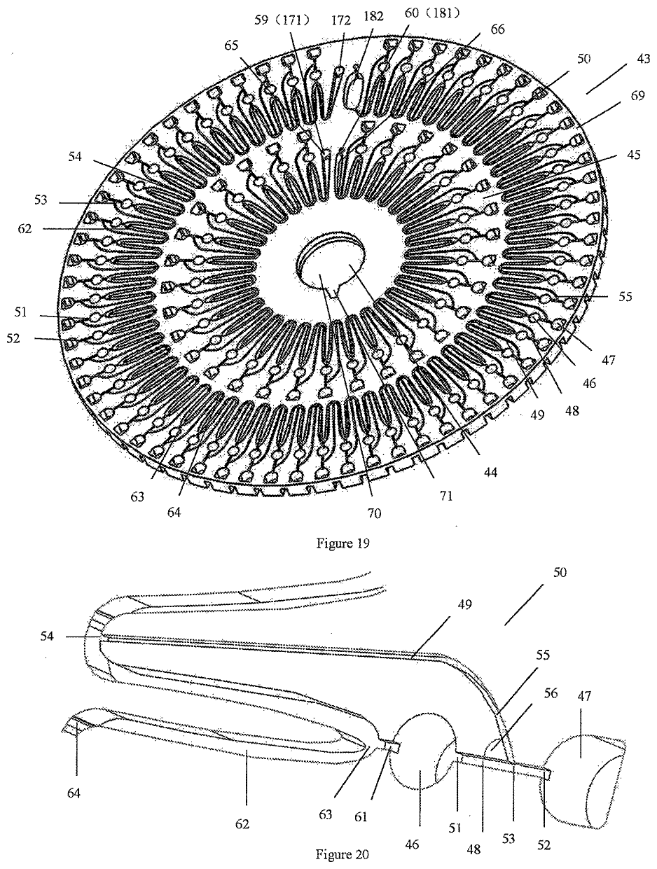

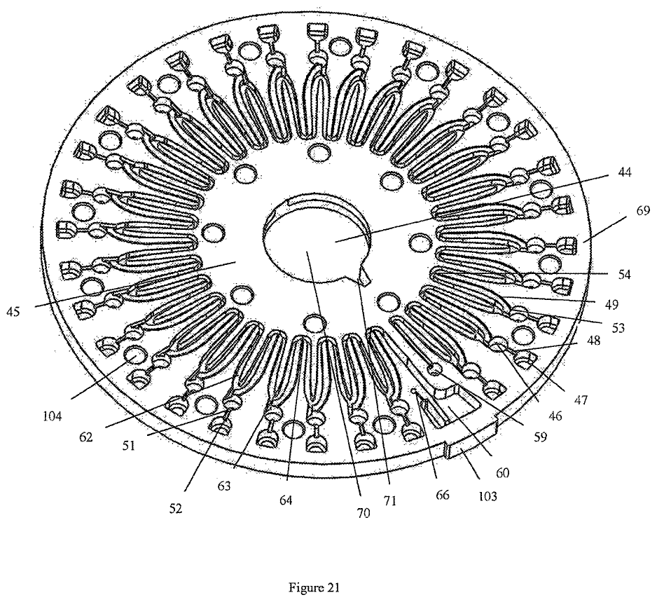

(2) Centrifugal Microfluidic Chip for Nucleic Acid Multiplex Detection with the Second Type of Exhaust Channel Setting (Corresponding to FIGS. 21-24)

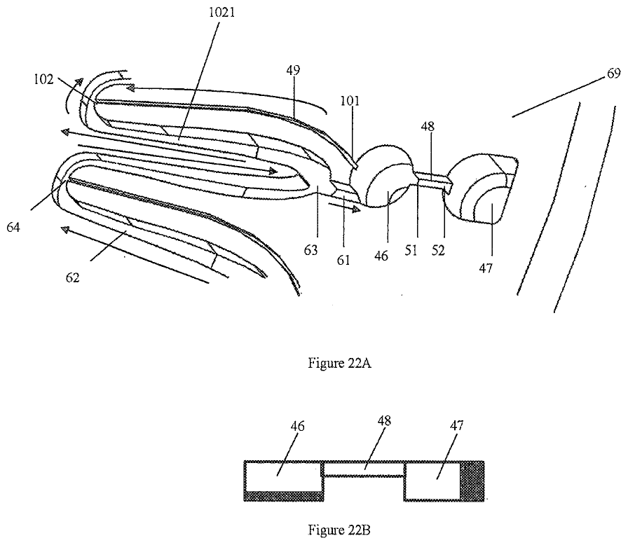

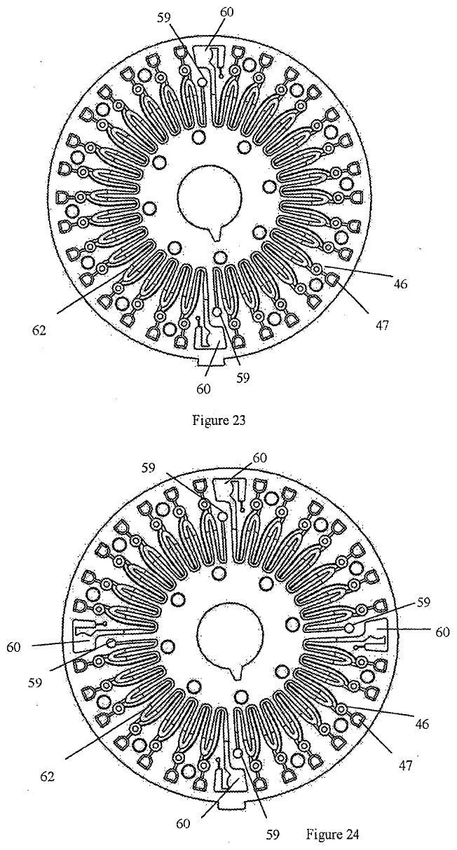

FIGS. 21-24 demonstrates other formats of centrifugal microfluidic chip for nucleic acid multiplex detection, which also have the second type of exhaust channel, where FIG. 21 is a front view of the chip; FIG. 22A is a detailed view of one detection unit 691; FIG. 22B is explanation on how the liquid 461 in the reaction chamber 46 enter into the detection chamber 47. FIG. 23 is the front view of a variety of FIG. 20, which is featured with two independent sections of detection and each section has 15 detection units. FIG. 24 is the front view of variety of FIG. 20, which is featured with four independent detection section, of each has 7 detection units. These chips are independent structural forms with unique design logic and application scenarios, realizing nucleic acid multiplex detection through the second type of exhaust channel setting (exhaust channel in communication with the main flow channel).

As shown in FIGS. 21-24, the overall structure of the centrifugal microfluidic chip 21 includes a circular chip body 69 with a rotation center 70 (consistent with claim 16). The rotation center 70 is a clamping groove 71 matching the rotating shaft of the centrifugal driving device, ensuring stable fixation during centrifugation and coaxial rotation with the driving device.

The chip body 69 adopts an integrated single-layer structure without sub-chips, and all functional components are integrated on the circular chip body 69. Multiple groups of detection units (7-30 detection unite groups optional) are arranged radially along the circular ring of the chip body 69, ensuring uniform distribution of centrifugal force on each detection unit 62. Each group of detection units includes a reaction chamber 46 (for nucleic acid amplification) and a detection chamber 47 (for sample detection to detect the copies of the nucleic acid or the product of the amplification in the reaction chamber 46), which are connected through a first fluid channel 48. A second channel 1021 is provided configured to receiving the liquid sample though the sample adding area 59, the sample adding area is also an opening or inlet of the second channel for inject a liquid sample into the second channel as to fill into the second channel 1021. If the second channel is filled with the liquid sample, the rest liquid sample will be flow into the liquid pool 60. In some embodiments, if all of the liquid sample entering into the reaction chambers (many reaction chambers is liquid communication with the second channel 1021, the air in the reaction chamber will be exhausted by the exhaust channel 49 into the second channel 1021. The second channel is air communication with the external atmospheric through the opening for receiving liquid sample or the outlet of the second channel that is communication with the liquid pool 60.

If many detections unite are connected with the second channel, each detection unites are independently fluid communication with the second channel 1021 for receiving the liquid sample. In some embodiments, each detection unites for detecting target nucleic acid are different, so, using one sample to enter into the second channel where each detecting unites receive the same liquid sample as enter into the reaction chamber and detection chamber. In some embodiments, the second channel is connected with the reaction chamber and the detection chamber by a exhaust channel for exhaust the air in the detection and reaction chamber when the liquid sample enters into the reaction chambre or detection chamber.

The exhaust channel 49 is led out from the reaction channel 46, and the combination of the fluid channel 48 and the exhaust channel 49 automatically provides resistance for the sample in the reaction chamber 46 to enter the detection chamber 47, realizing controllable transfer of the sample. The air is communication between the reaction chamber 47 and the second channel where the liquid sample enter or excites.

Here is the detail description for one detection unite 691 as shown FIGS. 22A and 20. In some embodiments, the detection unites including a first channel 48 which connect with reaction chamber 46 and the detection chamber 47. The liquid in the reaction chamber can enter into the detection chamber by the first channel 48. A second channel 1021 is provide as the main channel for receiving the liquid sample, each detection unite has a section of the channel. As in FIG. 22A, the second channel in this unite is stared from the point 64 to the point of 63 and then to the next point 102 (one part of the second channel 1021) in this detection unite 691, and the part of the second channel (64-63-102) has a reaction chamber 46 and the detection 47 and an air exhaust channel 49. When the liquid is flow into the second channel 1021 from the point 64 to the point 102 and filling the second channel 1021. As to control the liquid sample to enter into the reaction form the second channel, here provide a third channel 61 that connected the second channel 1021 and the reaction chamber 46. The size of the third channel 61 is less than the size of the second channel 1021. Due to surface tension, the inner diameter of the third channel 61 allows gas to pass through but prevents liquid to enter into from the second main channel. When the liquid is injected from the adding opening 59 for entering into the second channel and the air in the second channel is exhausted out from the washing pool 60 though the second channel.

In some embodiments, an air exhaust channel 49 is provide that keep the air communication between the reaction chamber 46/detection chamber 47 and the second channel 1021.

During use, the chip is placed horizontally with the sample-adding port facing upward, the clamping groove 71 is buckled with the rotating shaft of the detection device, the protrusion 103 on the outer edge of the chip body 69 points the notch of the chip holder of the chip amplification device.

Firstly, using inject device to inject the liquid sample into the second channel 1021 though the opening 59, all second channel 1021 is filled with a liquid sample, as to assure each detect unite can receive the liquids ample. Due to surface tension, the liquid sample cannot enter into the reaction chamber through the third channel 61. At this time, the chip is not drived by the centrifuge device.

For one detection unite 691 in FIG. 22A, a part of the second channel form point 64 to Piot 102 is filled with a liquid sample, the liquid is flowed from the point 64 (upstream) to the point 102 (downstream) as the direction indicated by the arrow (FIG. 22A). The point 64 is closed to the opening 59 for receiving the liquid sample, and the point 102 is the downstream of the point 64 this closed to the liquid pool as to exhaust the rest liquid sample and the air (all most of the liquid sample are entered into the reaction chamber), So, in the second channel, the opening 59 of the second channel is upstream of the point 64, and the point 102 is the downstream of the point 64, which is the upstream of the liquid pool 60. When the liquid sample is injected and flowed from the upstream point 64 to the downstream point 102, this liquid will not enter into the exhaust channel 49 and the reaction chamber 46.

Secondly, staring the centrifuge device as to let the chip to rotate in a low-speed centrifugation, like 500-1000 rpm (the maximum centrifugal speed does not exceed 3000 rpm). The liquid sample in the second channel 1021 be forced to enter into the reaction chamber 46 through the third channel 61. In a preferred embodiment, all of the liquid sample in the second channel 1021 is forced to enter into the reaction chamber 46 of each detection unite, as shown FIG. 24, the liquid sample in the second channel from the point 64 to 102 (10-50 ul or any volume of liquid sample that is defined by the second channel 1021 are all entered into the reaction chamber 46 and the second channel hao no liquid occupied. When the liquid in the second channel enters into the reaction chamber 46, the air in the reaction chamber can be exhausted into the second channel 1021 through the channel 49. Because the second channel is no liquid sample, and the air from the channel 49 is very easily to exhaust out of the chip through the washing pool that connect with an end of the second channel. In a preferred embodiment, the air exhaust channel 49 is air exhaust that can not exhaust the liquid from the reaction chamber 46. As to chivie the design, the size of the exhaust channel 49 is very small and can let the air to flow and cannot let the liquid (like the sample) to flow into the channel 49.

After a time, the amplification is completed in the reaction chamber 46 as to product the amplification products (like many copies of the target Nucleic Acid), and then staring the centrifuge device as to let the chip to rotate in a higher-speed centrifugation, like 1000-2000 rpm, the liquid in the reaction chamber 46 is forced into the detection chamber 47 through the first channel 48, and at the same time, the air in the detection chamber 47 can be exhausted into the reaction chamber 46 through the first channel 48 (FIG. 22B) and then exhausted from the reaction chamber 46 to the second channel 1021 though the exhaust channel 49. Owing to the relatively high rotational speed, the liquid transfer procedure is accomplished in a very short time, and the gas venting action is also performed rapidly. In addition, given the modest volume of the detection chamber, only a small quantity of gas is expelled, thereby facilitating efficient gas removal.

The detection device can provide a heat source to heat the reaction chamber 46 and read fluorescent signals generated by the sample reaction.

In the interval of every two detection units, there is a small round position letter area 104, in which an upper-case letter is printed to indicate the position of each units.

The key channel and chamber Design is as below:

First fluid channel 48: Connects the outlet 51 of the reaction chamber 46 and the inlet 52 of the detection chamber 47, with a width of 300 μm-500 μm and a depth of 250 μm-400 μm. The channel is designed as a gentle arc shape, reducing flow resistance while ensuring that the sample can only enter into the detection chamber 47 under specific centrifugal conditions (medium-speed centrifugation), and cannot flow into the detection chamber 47 during low-speed centrifugation.

Exhaust channel 49: Includes an inlet 101 (connection with the reaction chamber 46) and an outlet 102 (connected to the main flow channel (second channel) 62). The outlet 102 is closer to the rotation center 70 than the inlet 101, so the exhaust direction is opposite to the rotational centrifugal direction. The width of the exhaust channel 49 is 100 μm-200 μm, and the depth is 100 μm-200 μm, which is smaller than the second fluid channel 62, 1021, effectively preventing the liquid sample from entering the exhaust channel 49 during centrifugation. In a preferred embodiment, liquid cannot enter into the exhaust channel 49 but air can enter since the liquid surface tension.

Second channel as the main flow channel 62: Arranged in a circular ring shape coaxially with the chip body 69 and the rotation center 70, with a width of 600 μm-900 μm and a depth of 400 μm-550 μm. A single sample-adding port 59 is provided at the starting end of the main flow channel 62, and a waste liquid chamber 60 is provided at the tail end. The two ends of the main flow channel 62 are not connected, realizing sample filling and collection of excess samples.

Reaction chamber 46 and detection chamber 47: The reaction chamber 46 has a circular cross-section with a volume of 4 μL-8 μL, ensuring uniform mixing of the sample and amplification reagents without dead corners. The detection chamber 47 has an oval cross-section with a volume 1.3-1.6 times that of the reaction chamber 46, ensuring that all amplification products in the reaction chamber 46 can enter the detection chamber 47 without residue.

The core characteristics of this microfluidic chip includes:

The integrated single-layer structure without sub-chips makes the chip more compact and lightweight, suitable for small-batch multi-indicator detection of a single sample (such as screening of multiple respiratory pathogens in a single patient sample).

The radial arrangement of detection units 94 ensures that each reaction chamber 46 receives uniform centrifugal force, and the volume of the sample entering each reaction chamber 46 is consistent (error within ±4%), improving the repeatability of detection results.

The exhaust channel 49 is directly connected to the second circular ring-shaped main flow channel 62, forming a closed internal pressure balance system. It does not need to be connected to the external atmosphere, effectively avoiding aerosol contamination (consistent with the second setting mode of claim 17).

Nucleic acid multiplex detection can be completed with only two centrifugation steps, without high-speed centrifugation (the maximum centrifugal speed does not exceed 3000 rpm), avoiding insufficient dissolution and precipitation of pre-embedded freeze-dried reagents, and ensuring the stability of the reaction.

### Reagent Pre-Embedding and Material Selection:

The reaction chamber 46 is pre-embedded with solid nucleic acid amplification reagents (such as freeze-dried balls of ERA, RPA, or LAMP reagents), and the detection chamber 47 is pre-embedded with solid nucleic acid detection reagents (such as freeze-dried reagents based on CRISPR, Ago, or hybridization probe technology), which can be stored and transported at room temperature, avoiding cold-chain limitations.

The chip body 69 is made of materials with good biocompatibility and easy processing, such as PMMA (Polymethyl Methacrylate) or PC (Polycarbonate), and the channels and chambers are formed by laser etching technology, ensuring stable structural dimensions and low manufacturing costs.

Beside the full-disk chip format of this microfluidic chip, there are also other different varieties of this centrifugal microfluidic chip. In the full-disk chip format, the main fluid channel 62 is arranged as a continuous channel as a circular format on the whole chip body 69. Varieties can be made that two or more sets of U-shaped detection units with less numbers of detection units than the whole chip shown in FIG. 21. FIG. 23 and FIG. 24 show two varieties of this type of centrifugal microfluidic chips, with two and four sets of detection units containing 15 and 7 detection units respectively. These two types of chips can detect two and 4 samples on one single chip. Each set of detection units contains one sample inlet 59 and one waste liquid chamber 60 and work independently.

This type of chip includes, but not limited 1, 2 or 4 sets of detection units. One or more than one sets of detection units, with equal or different numbers of detection units in each section can be made and should be considered as varieties of this type of centrifugal microfluidic chip.

### Example 7: Usage Method of the Centrifugal Microfluidic Chip for Nucleic Acid Multiplex Detection

Usage Method of the Centrifugal Microfluidic Chip Provided in Examples 4 and 5 (First Setting Mode, Exhaust Channel Connected to the Outside)

The centrifugal microfluidic chips provided in Examples 4 and 5 can be applied to multiplex nucleic acid detection, including the following steps:

### Step 1: Sample Addition and Reconstitution

Add samples using a pipette. Aspirate different detection samples and inject them into the chip body 45 and the sub-chip 69 through the first sample-adding hole 171 and the second sample-adding hole 172 respectively. Under the action of the exhaust channel 49, the liquid is evenly distributed to the branch channels 61 along the main flow channel 58 in the chip body 45 and the sub-chip 69, and enters the reaction chamber 46 to reconstitute the solid amplification reagents in the reaction chamber 46, and stays in the reaction chamber 46 under the action of pressure balance;

### Step 2: Amplification Reaction

Place the uniformly mixed centrifugal microfluidic chip 43 in the detection device in parallel, and fix the clamping groove 70 with the rotating shaft in the detection device. At this time, the chip is heated, and the nucleic acid in the sample to be tested will undergo an amplification reaction at a certain temperature. The amplification reaction includes, but is not limited to, isothermal amplification reaction and PCR reaction;

### Step 3: Detection Reaction

After Step 2 is completed, start the centrifugal device. At this time, the centrifugal microfluidic chip 43 rotates clockwise (rotating speed: 800 rpm). The amplification product in the reaction chamber 46 flows into the corresponding detection chamber 47 under the action of centrifugal force, and mixes with the detection reagent to undergo a detection reaction. The detection reaction includes, but is not limited to, various detection systems established based on hybridization probe technology, CRISPR, Ago, RNaseH, etc.; during the transfer of the sample to be tested from the reaction chamber 46 and the detection chamber 47, the gas in the detection chamber 47 will enter the outside through the exhaust channel 49;

### Step 4: Data Reading

After Step 3 is completed, read the fluorescence intensity on the detection pipeline of the microfluidic chip through the optical device of the detection device to obtain the detection result.

Usage Method of the Centrifugal Microfluidic Chip Provided in Example 6 (Second Setting Mode, Exhaust Channel in Communication with the Main Flow Channel)

The centrifugal microfluidic chip provided in Example 6 can be applied to multiplex nucleic acid detection, including the following steps:

### Step 1: Sample Addition and Reconstitution

Add samples using a pipette. Aspirate different detection samples and add them to the first sample-adding hole 171 and the second sample-adding hole 172 respectively. Under the air pressure drive of the pipette, the sample to be tested will be evenly distributed along the main flow channel 58, and the excess liquid will enter the waste liquid pool 60; after the main flow channel 58 is completely filled, seal the sample-adding port with a transparent film; due to the difference in the pore size of the main flow channel 58 and the branch channel 61, the liquid cannot enter the reaction chamber 46;

### Step 2: Amplification Stage

Place the sealed centrifugal microfluidic chip 43 in parallel into the centrifugal driving device of the detection device, and perform low-speed clockwise centrifugation (rotating speed: 500 rpm). The sample to be tested is distributed from the main flow channel 58 to the reaction chamber 46; at this time, the chip is heated, and the nucleic acid in the sample to be tested will undergo an amplification reaction at a certain temperature;

### Step 3: Detection Reaction

After Step 2 is completed, start the centrifugal driving device of the detection device again and increase the centrifugal speed (rotating speed: 1500 rpm). The sample to be tested is driven by centrifugal force to transfer from the reaction chamber 46 to the corresponding detection chamber 47; after the amplification product is mixed with the detection reagent, a detection reaction will occur at a certain temperature. The detection reaction includes, but is not limited to, various detection systems established based on hybridization probe technology, CRISPR, Ago, RNaseH, etc.;

### Step 4: Data Reading

After Step 3 is completed, read the fluorescence intensity on the detection pipeline of the centrifugal microfluidic chip 43 through the optical device of the detection device to obtain the detection result.

According to detection needs, the fixed amplification reagents for nucleic acid amplification in the reaction chamber 46 and the detection substances for detection in the detection chamber 47 include freeze-dried balls, freeze-dried powders, or quality control internal standard reagents.

Both the amplification reaction and the detection reaction need to be carried out at a certain temperature. Therefore, in Step 2, a heating device is required to provide the centrifugal microfluidic chip 43 with a suitable temperature, for example, an isothermal amplification reaction needs to provide a reaction temperature of 30-65 °C.

### Example 8: Verification of Detection Effect of the Centrifugal Microfluidic Chip for Nucleic Acid Multiplex Detection

Genotyping Detection Using the Centrifugal Microfluidic Chip Provided in Example 4 (First Setting Mode, Exhaust Channel Connected to the Outside)

The ERA-CRISPR detection technology (enzymatic recombination amplification combined with CRISPR detection technology) was used to detect respiratory pathogen nucleic acids (influenza A virus, influenza B virus, respiratory syncytial virus, adenovirus, human rhinovirus, Mycoplasma pneumoniae, human metapneumovirus, parainfluenza virus) in blood samples of suspected respiratory infections, and the qPCR detecting method (commercial reagent) was used for comparative verification. The oral swab was lysed for nucleic acid using a sample release agent, and the obtained lysate was used as a template. The same amount of template was used for loading, and the microfluidic chip device and the qPCR detecting method were used to amplify and detect the samples respectively. For the ERA-CRISPR detection technology, ERA freeze-dried microspheres containing specific amplification primers were pre-installed in the amplification chamber of the chip device, and CRISPR detection system freeze-dried microspheres containing specific detection systems were pre-installed in the detection chamber. The presence of corresponding pathogenic microorganism nucleic acids in the samples was determined, and the results are shown in Table 1.

TABLE 1
Verification List of Detection Performance of the
Centrifugal Microfluidic Chip (First Setting Mode)

| | Type of | Centrifugal | qPCR |
|---|---|---|---|
| | Microorganism | Microfluidic | Detection |
| Sample ID | To be detected | Chip Device | Method |
| Sample 1 | Influenza A virus | −Negative | Negative |
| | Influenza B virus | −Negative | Negative |
| | Respiratory syncytial virus | −Negative | Negative |
| | Adenovirus | −Negative | Negative |
| | Human rhinovirus | −Negative | Negative |
| | Mycoplasma pneumoniae | +Positive | Positive |
| | Human metapneumovirus | −Negative | Negative |
| | Parainfluenza virus | −Negative | Negative |

It can be seen from Table 1 that under the same other conditions, the detection result of the centrifugal microfluidic chip device in Example 1 is consistent with that of the qPCR detecting method, but it is more convenient and fast to use, has lower comprehensive use cost, and is more suitable for on-site detection in the field and special environments.

Genotyping Detection Using the Centrifugal Microfluidic Chip Provided in Example 6 (Second Setting Mode, Exhaust Channel in Communication with the Main Flow Channel)

This example uses the centrifugal microfluidic chip provided in Example 6 (FIGS. 17-20) for genotyping detection. The ERA-CRISPR detection technology (enzymatic recombination amplification combined with CRISPR detection technology) was used to detect respiratory pathogen nucleic acids (influenza A virus, influenza B virus, respiratory syncytial virus, adenovirus, human rhinovirus, Mycoplasma pneumoniae, human metapneumovirus, parainfluenza virus) in blood samples of suspected respiratory infections, and the qPCR detection method (commercial reagent) was used for comparative verification. The oral swab was lysed for nucleic acid using a sample release agent, and the obtained lysate was used as a template. The same amount of template was used for loading, and the microfluidic chip device and the qPCR detection method were used to amplify and detect the samples respectively. For the ERA-CRISPR detection technology, ERA freeze-dried microspheres containing specific amplification primers were pre-installed in the amplification chamber of the chip device, and CRISPR detection system freeze-dried microspheres containing specific detection systems were pre-installed in the detection chamber. The presence of corresponding pathogenic microorganism nucleic acids in the samples was determined, and the results are shown in Table 2.

TABLE 2
Verification List of Detection Performance of the
Centrifugal Microfluidic Chip (Second Setting Mode)

| | Type of | Centrifugal | qPCR |
|---|---|---|---|
| | Microorganism | Microfluidic | Detection |
| Sample ID | To be detected | Chip Device | Method |
| Sample 1 | Influenza A virus | +Positive | Positive |
| | Influenza B virus | +Positive | Positive |
| | Respiratory syncytial virus | −Negative | Negative |
| | Adenovirus | −Negative | Negative |
| | Human rhinovirus | −Negative | Negative |
| | Mycoplasma pneumoniae | −Negative | Negative |
| | Human metapneumovirus | −Negative | Negative |
| | Parainfluenza virus | −Negative | Negative |

It can be seen from Table 2 that under the same other conditions, the detection result of the centrifugal microfluidic chip device in Example 3 is consistent with that of the qPCR detection method, but it is more convenient and faster to use, has lower comprehensive use cost, and is more suitable for on-site detection in the field and special environments.

At the same time, this example also conducted hundreds of detection experiments to comprehensively compare the detection result accuracy of the centrifugal microfluidic chips in Example 4 (first setting mode) and Example 6 (second setting mode). It was found that the accuracy of the centrifugal microfluidic chip in the first setting mode reached 98.1%, and the accuracy of the centrifugal microfluidic chip in the second setting mode reached 99.9%. The reason may be that the second setting mode can completely avoid aerosol contamination, so the accuracy is higher.

### Example 9: Structure of the Gravity-Driven Typing Chip

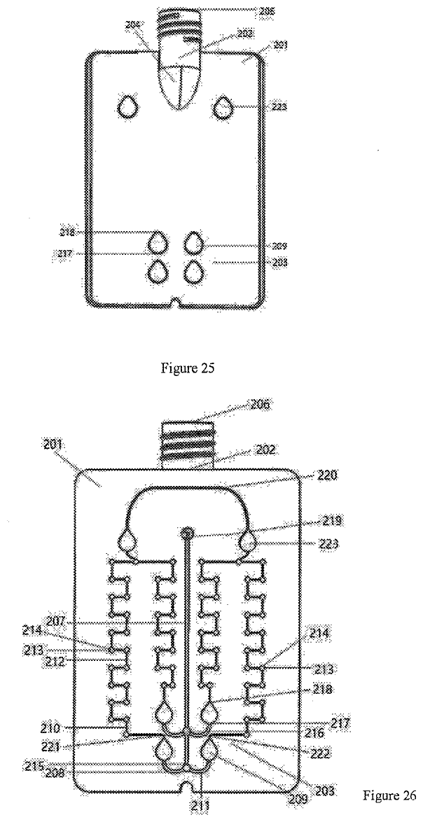

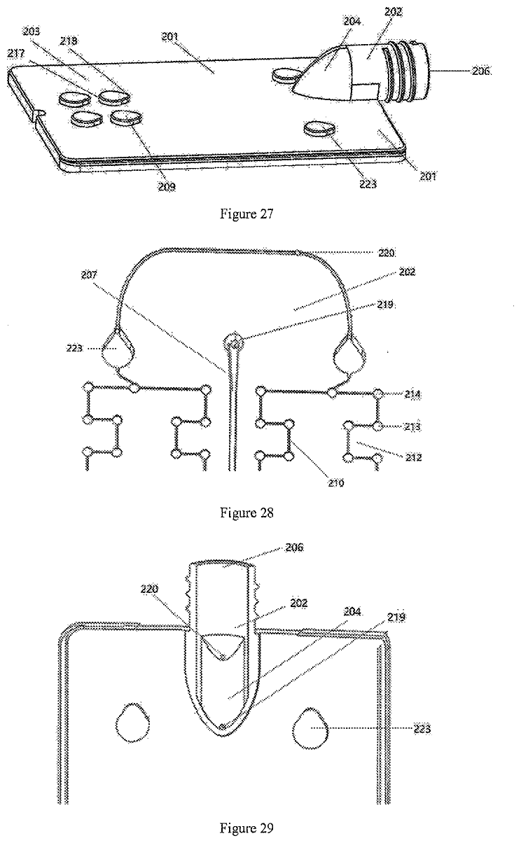

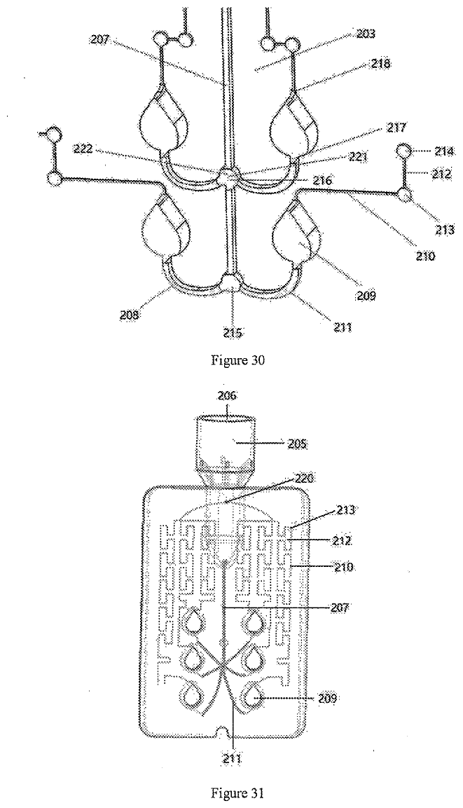

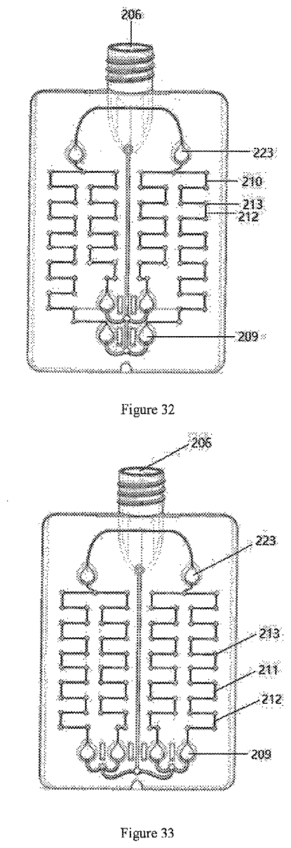

The structure of the gravity-driven typing chip provided in this example is shown in FIGS. 21-29, where FIG. 25 is a front view of the gravity-driven typing chip with four reaction chambers; FIG. 26 is a back view of the gravity-driven typing chip with four reaction chambers; FIG. 27 is a top view of the gravity-driven typing chip with four reaction chambers; FIG. 28 is a schematic structural diagram of the sample-adding area of the gravity-driven typing chip with four reaction chambers; FIG. 29 is a schematic cross-sectional structural diagram of the sample-adding area of the gravity-driven typing chip with four reaction chambers; FIG. 30 is a schematic structural diagram of the detection area of the gravity-driven typing chip with four reaction chambers; FIG. 31 is a structural diagram of the gravity-driven typing chip with six reaction chambers arranged radially; FIG. 32 is a structural diagram of the gravity-driven typing chip with four reaction chambers arranged radially; FIG. 33 is a structural diagram of the gravity-driven typing chip with four reaction chambers arranged horizontally.

As shown in FIGS. 21-26, the gravity-driven typing chip 201 provided in this example includes a sample-adding area 202 and a detection area 203. The sample-adding area 202 includes a liquid storage chamber 204. The liquid storage chamber 204 is used for containing the sample to be tested or pretreating the sample (such as amplifying the target nucleic acid or lysing the sample), and there is only one liquid storage chamber 204, which is cylindrical. When the sample is added to the microfluidic chip 201, it basically stays in the liquid storage chamber 204 first. The microfluidic chip 201 can also be equipped with a tube cap for sealing the microfluidic chip 201. The tube cap can be in the form of a plug. When the tube cap is tightened downward, the plug can provide downward pressure to prompt the sample near the sample-adding port 206 to completely flow into the liquid storage chamber 204, and make the sample in the liquid storage chamber 204 in a high-pressure state. The channels in the microfluidic chip 201 include a main flow channel 207 and a plurality of branch flow channels 208. The width of the flow channels is 400 μm-800 μm. In this example, the width of the main flow channel 207 and the branch flow channels 208 is preferably 400-600 μm, and the depth is 400-600 μm.

The detection area 203 can be provided with any number of groups of reaction chambers 209 according to the needs of project genotyping detection, which is used for sample processing and detection (such as for multiple genotyping detection of the same target nucleic acid or multiplex detection of different target nucleic acids). As shown in the gravity-driven microfluidic chip 201 in FIG. 25, six reaction chambers 209 are arranged, which are radially arranged in a multi-chamber center at one end of the main flow channel 207. The reaction chamber 209 is provided with an exhaust channel 210, and the exhaust channel 210 is connected to the liquid storage chamber 204. This design enables the reaction chamber 209 of the microfluidic chip 201 to be not connected to the external atmosphere. The exhaust channel 210 of the reaction chamber 209 is directly connected to the liquid storage chamber 204, and the atmosphere pressed out of the reaction chamber 209 is discharged back to the liquid storage chamber 204. At this time, the air pressure inside the microfluidic chip 201 is balanced, and the sample entering the reaction chamber 209 is also difficult to flow out of the reaction chamber 209, and backflow is difficult to occur. When the liquid flows in the microfluidic chip 201, no external driving is required, and it can be realized only by the gravity of the liquid itself.

As shown in FIG. 30, the reaction chamber 209 is further provided with a sample inlet channel 211. The pore size of the exhaust channel 210 is smaller than that of the sample inlet channel 211, and the exhaust channel adopts a multi-turn tortuous flow channel with a small pore size, so that the sample can flow into the reaction chamber 209 from the sample inlet channel 211 but cannot flow out of the reaction chamber 209 from the exhaust channel 210. The main flow channel 207 of the microfluidic chip 201 and the sample inlet channel 211 of the reaction chamber 209, which are used for sample flow, have a wide pore size, which can enable the liquid to flow smoothly. However, the pore size of the exhaust channel 210 of the reaction chamber 209 is significantly smaller than these flow channels. The exhaust channel 210 has a small pore size and can only allow gas to pass through and is not smooth for liquid discharge. Therefore, after the reaction chamber 209 is filled with the sample, the sample will be blocked when continuing to advance, and it is difficult to flow out from the exhaust channel 210 of the reaction chamber 209, and will remain in the reaction chamber 209 to complete the reaction and achieve accurate detection. The pore size of the exhaust channel 210 is smaller than 300 μm, which can play a certain role in blocking the liquid. Preferably, the pore size of the exhaust channel 210 is 50-300 μm. In this example, the pore size of the exhaust channel 210 is 100 μm.

As shown in FIGS. 21-26, the exhaust channel 210 is provided with at least one bent structure 212. The bent structure 212 can extend the length of the exhaust channel 210 as much as possible, so that there are more pipes to store the discharged gas, and prevent the situation that the pressure increases due to the large number of reaction chambers 209 and only one liquid storage chamber 204, which makes it difficult to fully accommodate all the gas. At the same time, the exhaust channel 210 is further provided with a small chamber 214 at the bent part 213 of the bent structure 212 for temporarily accommodating the excess discharged gas. Preferably, the bent structure 212 provided in the exhaust channel 210 is a back-and-forth bent channel. As shown in FIG. 28, before the exhaust channel 210 is connected to the liquid storage chamber 204, an exhaust chamber 223 can also be set for storing the excess discharged gas, and after the exhaust chamber 223 is filled, the gas continues to return to the liquid storage chamber 204.

The microfluidic chip 201 can be provided with a plurality of reaction chambers 209, which are respectively in fluid communication with the main flow channel 207 of the microfluidic chip 201 through the sample inlet channel 211, so as to be suitable for the detection of different target nucleic acids or different genotypes. As shown in the microfluidic chip 201 in FIG. 31, six reaction chambers 209 are provided; as shown in the microfluidic chips 201 in FIGS. 21-26, 28, and 29, four reaction chambers 209 are provided. The number of reaction chambers 209 can be selected according to the actual project detection needs. In the microfluidic chip 201, the reaction chambers 209 can be arranged in any way, such as horizontal arrangement, vertical arrangement, or radial arrangement at the end 215 of the main channel 207 in any other way. A plurality of branch ports 216 can also be set at the lower end of the main channel 207 for the arrangement of the reaction chambers 209. The reaction chambers 209 of the microfluidic chip 201 shown in FIGS. 27 and 28 are arranged in a radial form; the reaction chambers 209 of the microfluidic chip 201 shown in FIG. 33 are arranged in a horizontal form; the reaction chambers 209 of the microfluidic chip 201 shown in FIGS. 21-26 are arranged with two branch ports 216, and two reaction chambers 209 are arranged horizontally at each branch port.

The reaction chamber 209 is provided with a sample inlet 217 and an exhaust port 218. The sample inlet 217 is in fluid communication with the sample inlet channel 211, and the exhaust port 218 is in gas communication with the exhaust channel 210; the position of the exhaust port 218 is higher than that of the sample inlet 217. As shown in FIG. 29, the sample inlet 217 is located at the lower part of the reaction chamber 209, and the exhaust port 218 is located at the upper part of the reaction chamber 209. The purpose of this design is to ensure that after the sample enters the reaction chamber 209, it can smoothly exhaust upward, and at the same time, it can stay in the reaction chamber 209 more stably to prevent backflow. After the sample to be tested completes the reaction conducive to detection (such as reaction with detection reagents for signal amplification, etc.) in the reaction chamber 209, the result detection is directly carried out in the reaction chamber 209. One side of the reaction chamber 209 is made of a transparent film, and the detection instrument can detect the fluorescent substances generated by the reaction through the film and read the detection result.

As shown in FIG. 29, the liquid storage chamber 204 is provided with a liquid outlet 219 and a gas inlet 220. The liquid outlet 219 is in fluid communication with the main flow channel 207, and the gas inlet 220 is in gas communication with the exhaust channel 210 of the reaction chamber 207; the position of the gas inlet 220 is higher than that of the liquid outlet 219. The position of the gas inlet 220 in the liquid storage chamber 204 is higher than that of the liquid outlet 219, which can ensure that all samples in the liquid storage chamber 204 are discharged smoothly and prevent interference from the incoming gas.

As shown in FIG. 30, a valve block cavity 222 is arranged between the liquid storage chamber 204 and the reaction chamber 209 for placing a solid valve block 221, which is used for controlling the fluid communication state between the liquid storage chamber 204 and the reaction chamber 209; the valve block 221 has two states: solid and liquid; when the valve block 221 is solid, the liquid storage chamber 204 and the reaction chamber 209 cannot be in fluid communication; when the valve block 221 is liquid, the liquid storage chamber 204 and the reaction chamber 209 are in fluid communication. At the same time, when the valve block 221 is solid, it is located in the main flow channel 207 connecting the liquid storage chamber 204 and the reaction chamber 209 to prevent the sample to be tested in the liquid storage chamber 204 from flowing into the reaction chamber 209; when the valve block 221 is liquid, it will flow to the exhaust channel 210 of the reaction chamber 209; the valve block 221 is paraffin or lipid substance (such as n-octadecane, stearic acid, palmitic acid, etc.), which is solid at room temperature and melts into liquid after heating; the density of the valve block 221 is less than that of water. When the valve block 221 is converted into a liquid state, it can always float on the top of the sample to be tested, which helps to condense at the exhaust port 218 at the top of the reaction chamber 209. If the density of the valve block 221 is greater than that of water, when the valve block 221 is converted into a liquid state and flows into the reaction chamber 209 together with the sample, it is easy to accumulate at the sample inlet 217 of the reaction chamber 209, leading to blockage of the sample inlet 217. Therefore, the density of the valve block 221 must be less than that of water. In this example, paraffin is preferably used as the valve block 221. Paraffin can achieve phase change at a specific temperature threshold between 40-90 °C. In this example, paraffin (manufacturer: Sigma-Aldrich, model: 411663) undergoes phase change at about 65 °C.

Since the microfluidic chip 201 is provided with a plurality of reaction chambers 209, and the sample inlets 217 of the reaction chambers 209 are gathered together, sample backflow is prone to occur, causing the sample to overflow from one reaction chamber 209 and enter another reaction chamber 209, resulting in cross-influence. Therefore, how to prevent sample backflow and ensure the accuracy of detection results is very important. By arranging the exhaust channel 211 in the reaction chamber 209 and arranging the valve block 221 between the liquid storage chamber 204 and the reaction chamber 209, the blocking and communication between the liquid storage chamber 204 and the reaction chamber 209 are realized by using the temperature-induced phase change property of the valve block 221. After the valve block 221 is heated and melted, the flow channels between the liquid storage chamber 204 and each reaction chamber 209 are connected, and the sample in the liquid storage chamber 204 flows into each reaction chamber 209. The melted valve block 221 has a low density and will float on the top of the sample, flow into the reaction chamber 209 along with the sample, and condense near the exhaust port 218 of the reaction chamber 209 to block the exhaust channel 210 (since the sample volume is quantitative, it can at least reach near the exhaust port 218 of the reaction chamber 209). This also helps the sample to be tested stay more stably in the reaction chamber 209 after entering the reaction chamber 209, making backflow difficult to occur and preventing it from flowing to other chambers, thus ensuring that each step of the reaction can be completed smoothly until the final accurate detection is realized.

Preferably, the cross-section of the reaction chamber 209 is drop-shaped (FIG. 30), and the exhaust port 218 is located at the top of the drop shape, which helps the liquid paraffin to collect upward, making it easier to condense near the exhaust port 218.

The solid reagents pre-embedded in the liquid storage chamber 204 and the reaction chamber 209 can be flexibly set. Different reaction reagents are pre-embedded in different chambers. For example, when the microfluidic chip 201 provided in this example is used for genotyping detection of the target nucleic acid, the liquid storage chamber 204 is used for amplifying the target nucleic acid, and nucleic acid amplification reagents are pre-embedded in the liquid storage chamber 204; the reaction chamber 209 is used for detecting different genotypes of the target nucleic acid, and detection reagents for different genotypes are pre-embedded in the reaction chamber 209. The liquid storage chamber 204 is used for amplifying the target nucleic acid, and nucleic acid amplification reagents need to be pre-embedded in the liquid storage chamber 204. The reagents used for lysis are added from the sample-adding port 206 during sample detection; the nucleic acid amplification reagents are solid amplification reagents prepared by freeze-drying or drying, including but not limited to spherical, powdery, flaky, or blocky preparations in the form of freeze-drying, drying, air-drying, etc. The involved amplification reactions include, but are not limited to, RPA (Recombinase Polymerase Amplification), ERA (Enzymatic Recombinase Amplification), LAMP (Loop-mediated Isothermal Amplification), NEAR (Nicking enzyme-Assisted Reaction), NASBA (Nuclear acid sequence-based amplification), HDA (helicase-dependent amplification), TMA (transcription mediated amplification), SAT (Simultaneous Amplification and Testing), etc. The reaction chamber 209 is used for the fluorescent detection reaction between the amplified sample and the detection reagent. Detection reagents need to be pre-embedded in the reaction chamber 209. The detection reagents are solid detection reagents, including but not limited to spherical, powdery, flaky, or blocky preparations in the form of freeze-drying, drying, air-drying, etc. The involved detection reactions include, but are not limited to, various probe detection systems established based on RPA (Recombinase Polymerase Amplification), ERA (Enzymatic Recombinase Amplification), LAMP (Loop-mediated Isothermal Amplification), NEAR (Nicking enzyme-Assisted Reaction), NASBA (Nuclear acid sequence-based amplification), HDA (helicase-dependent amplification), TMA (transcription mediated amplification), SAT (Simultaneous Amplification and Testing), hybridization probe technology, CRISPR, Ago, RNaseH, etc. The detection results include, but are not limited to, fluorescent detection reactions and test strip visualization detection methods according to different actual needs. When the microfluidic chip 201 provided in this example is used for multiplex detection of target nucleic acids, the liquid storage chamber 204 is used for adding sample nucleic acid lysates, and solid detection reagents for amplification and detection of different nucleic acids need to be pre-embedded in the reaction chamber 209, which specifically include detection primers, probes, and detection premix. The detection premix includes enzymes and buffers required for detection.

The chip body structure of the microfluidic chip 201 uses materials with low cost, easy processing performance, and good biocompatibility for chips, such as PMMA (Polymethyl Methacrylate), PP (Polypropylene), PC (Polycarbonate), etc., as the substrate. Channels and reaction tanks are directly made by laser etching technology, or structures are etched on the PMMA substrate, and then PDMS is used for reverse molding.

During the use of the microfluidic chip 201, it needs to be kept in a vertical state, with the sample-adding port 206 facing upward. When in use, the microfluidic chip 201 can be inserted into a matching detection device, so that the microfluidic chip 201 is placed vertically with the sample-adding port 206 facing upward, and the detection device can provide a heat source to heat specific positions of the microfluidic chip 1 (such as the reaction chamber 209, valve block 221, etc.); it can also detect the fluorescent substances generated after the sample reaction in the microfluidic chip 1 through the detection device and read the detection result.

The microfluidic chip 201 provided in this example, through the clever design of flow channel connection and chamber position, does not require additional driving. The liquid can realize the sequential and independent amplification and detection reactions of the sample to be tested according to its own gravity. The whole process is easy to operate and has a fast reaction speed, and can effectively realize high-throughput detection of pathogens; in addition, the amplification/detection reagents of the microfluidic chip are all dry reagents, which can realize the room-temperature storage and transportation of the chip, avoiding the limitations of cold-chain transportation and −20 °C storage.

### Example 10: Usage Method of the Gravity-Driven Typing Chip

The gravity-driven microfluidic chip provided in Example 9 can be applied to various nucleic acid detection methods.

Method 1: Taking the genotyping detection based on enzyme-catalyzed isothermal amplification technology as an example, the usage method of the microfluidic chip device is illustrated:
Transfer: Add the sample to be tested containing lysis solution into the liquid storage chamber 204 of the microfluidic chip from the sample-adding port 206. The sample to be tested reconstitutes the solid amplification reagents in the liquid storage chamber 204. At this time, tighten the tube cap to completely seal the chip;
Reaction: Heat the chip. After the temperature rises to a certain level, an amplification reaction occurs in the liquid storage chamber 204. The amplification reaction includes, but is not limited to, isothermal amplification reaction and PCR reaction; while the amplification reaction is in progress, the valve block 221 blocked between the liquid storage chamber 204 and the reaction chamber 209 is also continuously melted. After the valve block 221 is completely melted, the pressure balance is achieved in the system, and the amplification product will move to the reaction chamber 209 under the action of gravity.

Read the result: After the reaction is completed, read the fluorescent signal through the device to obtain the detection result.

Method 2: Taking the multiplex detection based on enzyme-catalyzed isothermal amplification technology as an example, the usage method of the microfluidic chip device is illustrated:
Transfer: Add the sample to be tested containing lysis solution into the liquid storage chamber 204 of the microfluidic chip from the sample-adding port 206, and tighten the tube cap to seal the chip.
Reaction: Heat the chip. After the temperature rises to a certain level, the valve block 221 blocked between the liquid storage chamber 204 and the detection chamber 209 is also continuously melted. After the valve block 221 is completely melted, the pressure balance is achieved in the system, and the sample lysate will move to the reaction chamber 209 under the action of gravity; at the same time, the reagents in the reaction chamber 209 are dissolved and an amplification reaction occurs. The amplification reaction includes, but is not limited to, isothermal amplification reaction and PCR reaction;
Read the result: After the reaction is completed, read the fluorescent signal through the device to obtain the detection result.

### Example 11: Verification of Genotyping Detection Effect of the Gravity-Driven Typing Chip

This example uses the gravity-driven microfluidic chip provided in Example 9 (FIG. 25) for genotyping detection. The ERA-CRISPR detection technology (enzymatic recombination amplification combined with CRISPR detection technology) was used to detect Plasmodium nucleic acids (Plasmodium Spp., Plasmodium F., Plasmodium V., Plasmodium K.) in blood samples of people suspected of Plasmodium infection, and the qPCR detection method (commercial reagent) was used for comparative verification. The blood samples were lysed for nucleic acid using a sample release agent, and the obtained lysate was used as a template. The same amount of template was used for loading, and the gravity-driven microfluidic chip device and the qPCR detection method were used to amplify and detect the samples respectively. For the ERA-CRISPR detection technology, ERA freeze-dried microspheres containing specific amplification primers were pre-installed in the liquid storage chamber of the chip device, and CRISPR detection system freeze-dried microspheres containing specific detection systems were pre-installed in the reaction chamber. The presence of corresponding pathogenic microorganism nucleic acids in the samples was determined, and the results are shown in Table 3.

TABLE 3
Verification Results of Detection Performance
of the Gravity-Driven Typing Chip

| | Type of | Gravity-Driven | qPCR |
|---|---|---|---|
| | Microorganism | Microfluidic | Detection |
| Sample ID | to be detected | Chip Device | Method |
| Sample 1 | Plasmodium Spp. | +Positive | Positive |
| | Plasmodium F. | +Positive | Positive |
| | Plasmodium V. | −Negative | Negative |
| | Plasmodium K. | −Negative | Negative |
| Sample 2 | Plasmodium Spp. | −Negative | Negative |
| | Plasmodium F. | −Negative | Negative |
| | Plasmodium V. | −Negative | Negative |
| | Plasmodium K. | −Negative | Negative |
| Sample 3 | Plasmodium Spp. | +Positive | Positive |
| | Plasmodium F. | −Negative | Negative |
| | Plasmodium V. | +Positive | Positive |
| | Plasmodium K. | −Negative | Negative |

It can be seen from Table 3 that under the same other conditions, the detection result of the gravity-driven typing chip device in Example 9 is consistent with that of the qPCR detection method, but it is more convenient and fast to use, and is more suitable for on-site detection in the field and special environments.

### Example 12: Verification of Multiplex Detection Effect of the Gravity-Driven Typing Chip

This example uses the gravity-driven microfluidic chip device provided in Example 9 (FIG. 30) for multiplex detection. The ERA detection technology (enzymatic recombination amplification detection technology) was used to detect nucleic acids in suspected infected shrimp samples (White Spot Syndrome Virus (WSSV), Infectious Hypodermal and Hematopoietic Necrosis Virus (IHHNV), Acute Hepatopancreatic Necrosis Disease (AHPND/EMS) pathogen, Shrimp Hemocyte Iridescent Virus (SHIV), Enterocytozoon Hepatopenaei (EHP)), and the qPCR detection method (commercial reagent) was used for comparative verification. The shrimp meat crushing samples were lysed for nucleic acid using a sample release agent, and the obtained lysate was used as a template. The same amount of template was used for loading, and the gravity-driven microfluidic chip device and the qPCR detection method were used to amplify and detect the samples respectively. For the ERA detection technology, ERA amplification and detection system freeze-dried microspheres containing specific amplification primers and detection probes were pre-installed in the reaction chamber of the gravity-driven microfluidic chip device. After the reaction, the result was read to determine whether the sample contained the corresponding pathogenic microorganism nucleic acid, and the results are shown in Table 4.

TABLE 4
Verification List of Detection Performance
of the Gravity-Driven Typing Chip.

| | | Gravity-Driven | qPCR |
|---|---|---|---|
| Serial | | Microfluidic | Detection |
| Number | Type of Microorganism | Chip Device | Method |
| 1 | White Spot Syndrome Virus | +Positive | Positive |
| | (WSSV) | | |
| 2 | Infectious Hypodermal and | −Negative | Negative |
| | Hematopoietic Necrosis Virus | | |
| | (IHHNV) | | |
| 3 | Shrimp Hemocyte Iridescent | −Negative | Negative |
| | Virus (SHIV) | | |
| 4 | Enterocytozoon Hepatopenaei | −Negative | Negative |
| | (EHP) | | |

It can be seen from Table 4 that under the same other conditions, the detection result of the gravity-driven microfluidic chip device in Example 9 is consistent with that of the qPCR detection method, and it is more convenient and fast to use, and is more suitable for on-site detection in the field and special environments.

### Example 13: Structure of the Asymmetric Pressure-Driven Typing Chip

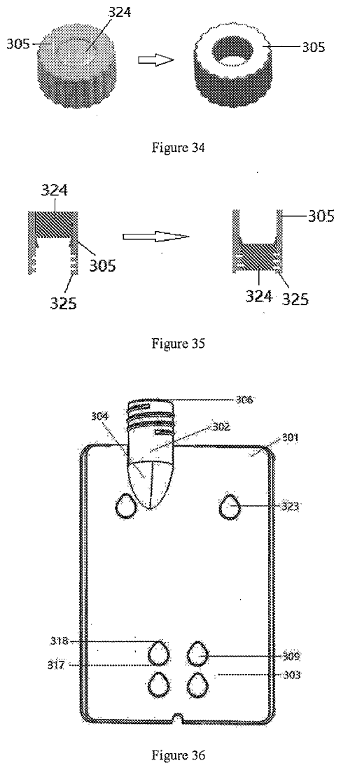

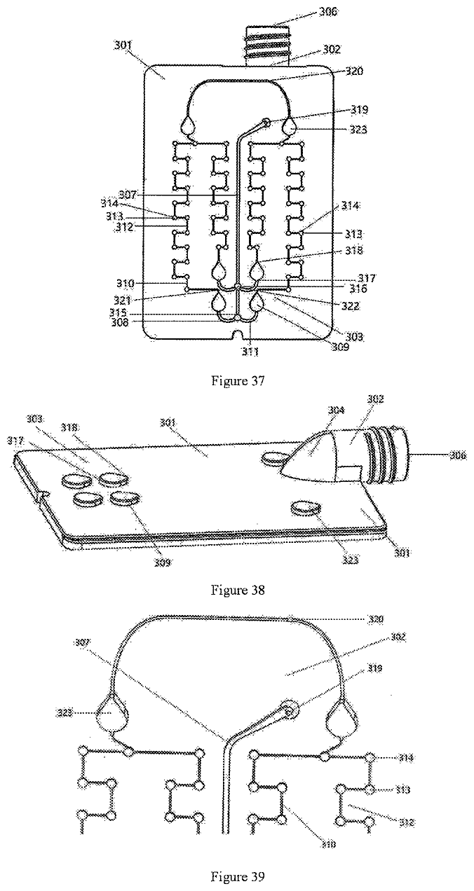

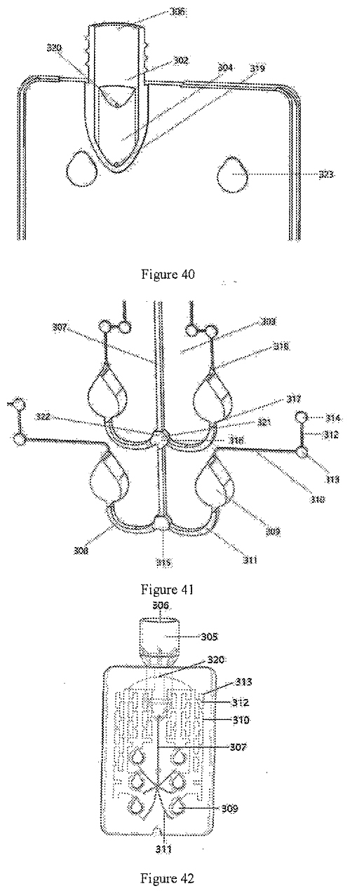

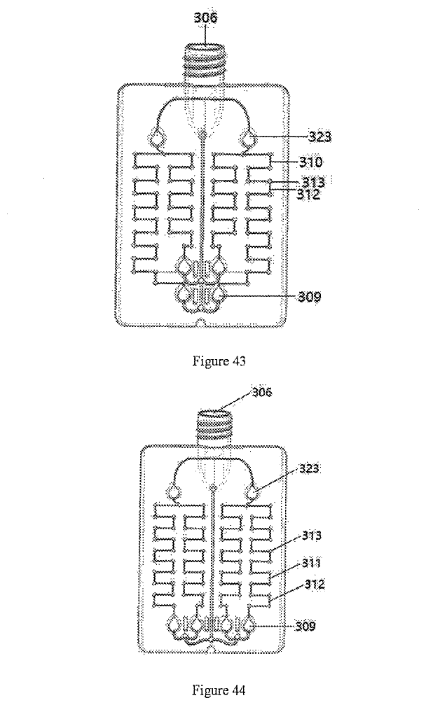

The structure of the asymmetric pressure-driven microfluidic chip provided in this example is shown in FIGS. 30-40, where FIG. 34 is a structural diagram of the tube cap of the asymmetric pressure-driven microfluidic chip; FIG. 35 is a schematic cross-sectional structural diagram of the tube cap of the asymmetric pressure-driven microfluidic chip; FIG. 36 is a front view of the asymmetric pressure-driven microfluidic chip with four reaction chambers; FIG. 37 is a back view of the asymmetric pressure-driven microfluidic chip with four reaction chambers; FIG. 38 is a top view of the asymmetric pressure-driven microfluidic chip with four reaction chambers; FIG. 39 is a schematic structural diagram of the sample-adding area of the asymmetric pressure-driven microfluidic chip with four reaction chambers; FIG. 40 is a schematic cross-sectional structural diagram of the sample-adding area of the asymmetric pressure-driven microfluidic chip with four reaction chambers; FIG. 41 is a schematic structural diagram of the detection area of the asymmetric pressure-driven microfluidic chip with four reaction chambers; FIG. 42 is a structural diagram of the asymmetric pressure-driven microfluidic chip with six reaction chambers arranged radially; FIG. 43 is a structural diagram of the asymmetric pressure-driven microfluidic chip with four reaction chambers arranged radially; FIG. 44 is a structural diagram of the asymmetric pressure-driven microfluidic chip with four reaction chambers arranged horizontally.

As shown in FIGS. 30-37, the asymmetric pressure-driven microfluidic chip 301 provided in this example includes a sample-adding area 302 and a detection area 303. The sample-adding area 302 includes a liquid storage chamber 304 and a tube cap 305. The liquid storage chamber 304 is used for containing the sample to be tested or pretreating the sample (such as amplifying the target nucleic acid or lysing the sample), and there is only one liquid storage chamber 304, which is cylindrical. When the sample is added to the microfluidic chip 301, it basically stays in the liquid storage chamber 304 first. The tube cap 305 plays the role of sealing the microfluidic chip 301 and applying pressure to the inside of the microfluidic chip 301 by pressing or screwing. The tube cap 305 can be in the form of a plug or a spiral. When the tube cap 305 is tightened downward, it not only plays a sealing role on the chip, but also the plug can provide downward instantaneous pressure to prompt the sample near the sample-adding port 306 to completely flow into the liquid storage chamber 304. Since the exhaust channel 310 is a multi-turn tortuous flow channel with a small pore size and the pore size of the exhaust channel 310 is smaller than that of the sample inlet channel 311, instantaneous asymmetric pressure is formed under the pressure applied by screwing or pressing the tube cap 305, and the sample in the liquid storage chamber 304 is transferred to the detection area 303 through the flow channel. The channels in the microfluidic chip 301 include a main flow channel 307 and a plurality of branch flow channels 308.

As shown in FIGS. 30 and 31, the top of the tube cap 305 provided in this example is provided with a pushable airbag 324. After closing the microfluidic chip 301, pushing the airbag 324 downward moves the airbag 324 from the top of the tube cap 305 to a position close to the spiral 325 of the tube cap, which can apply pressure to the inside of the chip to generate asymmetric pressure inside the chip and promote the sample to flow from the liquid storage chamber 304 into the reaction chamber 309. The tube cap 305 can also be detachably connected to the liquid storage chamber 304, and while sealing the system, it provides air pressure for the chip system to drive the liquid in the liquid storage chamber 304 to transfer to the reaction chamber 309. The main flow channel 307 of the microfluidic chip 301 and the sample inlet channel 311 of the reaction chamber 309, which are used for sample flow, have pore sizes set to enable the liquid to flow smoothly.

The detection area 303 can be provided with any number of groups of reaction chambers 309 according to the needs of project genotyping detection, which is used for sample processing and detection (such as for multiple genotyping detection of the same target nucleic acid or multiplex detection of different target nucleic acids). As shown in the asymmetric pressure-driven microfluidic chip 301 in FIG. 41, six reaction chambers 309 are arranged, which are radially arranged in a multi-chamber center at one end of the main flow channel 307. The reaction chamber 309 is provided with an exhaust channel 310, and the exhaust channel 310 is connected to the liquid storage chamber 304. This design enables the reaction chamber 309 of the microfluidic chip 301 to be not connected to the external atmosphere. The exhaust channel 310 of the reaction chamber 309 is directly connected to the liquid storage chamber 304, and the atmosphere pressed out of the reaction chamber 309 is discharged back to the liquid storage chamber 304. At this time, the air pressure inside the microfluidic chip 301 is balanced, and the sample entering the reaction chamber 309 is also difficult to flow out of the reaction chamber 309, and backflow is difficult to occur.

As shown in FIG. 41, the reaction chamber 309 is further provided with a sample inlet channel 311. The pore size of the sample inlet channel 311 is significantly larger than that of the exhaust channel 310, and the exhaust channel adopts a multi-turn tortuous flow channel with a small pore size. Under the pressure applied by screwing or pressing the tube cap 305, instantaneous asymmetric pressure is formed, so that the sample can flow into the reaction chamber 309 from the sample inlet channel 311 but cannot flow out of the reaction chamber 309 from the exhaust channel 310. The main flow channel 307 of the microfluidic chip 301 and the sample inlet channel 311 of the reaction chamber 309, which are used for sample flow, have a wide pore size, which can enable the liquid to flow smoothly. However, the pore size of the exhaust channel 310 of the reaction chamber 309 is significantly smaller than these flow channels. The exhaust channel 310 has a small pore size and can only allow gas to pass through and is not smooth for liquid discharge. Therefore, after the reaction chamber 309 is filled with the sample, the sample will be blocked when continuing to advance, and it is difficult to flow out from the exhaust channel 310 of the reaction chamber 309, and will remain in the reaction chamber 309 to complete the reaction and achieve accurate detection.

As shown in FIGS. 30-37, the exhaust channel 310 is provided with at least one bent structure 312. The bent structure 312 can extend the length of the exhaust channel 310 as much as possible, so that there are more pipes to store the discharged gas, and prevent the situation that the pressure increases due to the large number of reaction chambers 309 and only one liquid storage chamber 304, which makes it difficult to fully accommodate all the gas. At the same time, the exhaust channel 310 is further provided with a small chamber 314 at the bent part 313 of the bent structure 312 for temporarily accommodating the excess discharged gas. Preferably, the bent structure 312 provided in the exhaust channel 310 is a back-and-forth bent channel. As shown in FIG. 38, before the exhaust channel 310 is connected to the liquid storage chamber 304, an exhaust chamber 323 can also be set for storing the excess discharged gas, and after the exhaust chamber 323 is filled, the gas continues to return to the liquid storage chamber 304.

The microfluidic chip 301 can be provided with a plurality of reaction chambers 309, which are respectively in fluid communication with the main flow channel 307 of the microfluidic chip 301 through the sample inlet channel 311, so as to be suitable for the detection of different target nucleic acids or different genotypes. As shown in the microfluidic chips 301 in FIGS. 30 and 37, six reaction chambers 309 are provided; as shown in the microfluidic chips 301 in FIGS. 31-36, 38, and 40, four reaction chambers 309 are provided. The number of reaction chambers 309 can be selected according to the actual project detection needs. In the microfluidic chip 301, the reaction chambers 309 can be arranged in any way, such as horizontal arrangement, vertical arrangement, or radial arrangement at the end 315 of the main channel 307 in any other way. A plurality of branch ports 316 can also be set at the lower end of the main channel 307 for the arrangement of the reaction chambers 309. The reaction chambers 309 of the microfluidic chip 301 shown in FIGS. 37 and 38 are arranged in a radial form; the reaction chambers 309 of the microfluidic chip 301 shown in FIGS. 30 and 40 are arranged in a horizontal form; the reaction chambers 309 of the microfluidic chip 301 shown in FIGS. 31-36 are arranged with two branch ports 316, and two reaction chambers 309 are arranged horizontally at each branch port.

The reaction chamber 309 is provided with a sample inlet 317 and an exhaust port 318. The sample inlet 317 is in fluid communication with the sample inlet channel 311, and the exhaust port 318 is in gas communication with the exhaust channel 310; the position of the exhaust port 318 is higher than that of the sample inlet 317. As shown in FIG. 39, the sample inlet 317 is located at the lower part of the reaction chamber 309, and the exhaust port 318 is located at the upper part of the reaction chamber 309. The purpose of this design is to ensure that after the sample enters the reaction chamber 309, it can smoothly exhaust upward, and at the same time, it can stay in the reaction chamber 309 more stably to prevent backflow. After the sample to be tested completes the reaction conducive to detection (such as reaction with detection reagents for signal amplification, etc.) in the reaction chamber 309, the result detection is directly carried out in the reaction chamber 309. One side of the reaction chamber 309 is made of a transparent film, and the detection instrument can detect the fluorescent substances generated by the reaction through the film and read the detection result.

As shown in FIG. 40, the liquid storage chamber 304 is provided with a liquid outlet 319 and a gas inlet 320. The liquid outlet 319 is in fluid communication with the main flow channel 307, and the gas inlet 320 is in gas communication with the exhaust channel 310 of the reaction chamber 307; the position of the gas inlet 320 is higher than that of the liquid outlet 319. The position of the gas inlet 320 in the liquid storage chamber 304 is higher than that of the liquid outlet 319, which can ensure that all samples in the liquid storage chamber 304 are discharged smoothly and prevent interference from the incoming gas.

As shown in FIG. 41, a valve block cavity 322 is arranged between the liquid storage chamber 304 and the reaction chamber 309 for placing a solid valve block 321, which is used for controlling the fluid communication state between the liquid storage chamber 304 and the reaction chamber 309; the valve block 321 has two states: solid and liquid; when the valve block 321 is solid, the liquid storage chamber 304 and the reaction chamber 309 cannot be in fluid communication; when the valve block 321 is liquid, the liquid storage chamber 304 and the reaction chamber 309 are in fluid communication. At the same time, when the valve block 321 is solid, it is located in the main flow channel 307 connecting the liquid storage chamber 304 and the reaction chamber 309 to prevent the sample to be tested in the liquid storage chamber 304 from flowing into the reaction chamber 309; when the valve block 321 is liquid, it will flow to the exhaust channel 310 of the reaction chamber 309; the valve block 321 is paraffin or lipid substance (such as n-octadecane, stearic acid, palmitic acid, etc.), which is solid at room temperature and melts into liquid after heating; the density of the valve block 321 is less than that of water. When the valve block 321 is converted into a liquid state, it can always float on the top of the sample to be tested, which helps to condense at the exhaust port 318 at the top of the reaction chamber 309. If the density of the valve block 321 is greater than that of water, when the valve block 321 is converted into a liquid state and flows into the reaction chamber 309 together with the sample, it is easy to accumulate at the sample inlet 317 of the reaction chamber 309, leading to blockage of the sample inlet 317. Therefore, the density of the valve block 321 must be less than that of water. In this example, paraffin is preferably used as the valve block 321. Paraffin can achieve phase change at a specific temperature threshold between 40-90 °C. In this example, paraffin (manufacturer: Sigma-Aldrich, model: 411663) undergoes phase change at about 65 °C.

Since the microfluidic chip 301 is provided with a plurality of reaction chambers 309, and the sample inlets 317 of the reaction chambers 309 are gathered together, sample backflow is prone to occur, causing the sample to overflow from one reaction chamber 9 and enter another reaction chamber 309, resulting in cross-influence. Therefore, how to prevent sample backflow and ensure the accuracy of detection results is very important. By arranging the exhaust channel 310 in the reaction chamber 309 and arranging the valve block 321 between the liquid storage chamber 304 and the reaction chamber 309, the blocking and communication between the liquid storage chamber 304 and the reaction chamber 309 are realized by using the temperature-induced phase change property of the valve block 321. After the valve block 321 is heated and melted, the flow channels between the liquid storage chamber 304 and each reaction chamber 309 are connected, and the sample in the liquid storage chamber 304 flows into each reaction chamber 309. The melted valve block 321 has a low density and will float on the top of the sample, flow into the reaction chamber 309 along with the sample, and condense near the exhaust port 318 of the reaction chamber 309 to block the exhaust channel 310 (since the sample volume is quantitative, it can at least reach near the exhaust port 318 of the reaction chamber 309). This also helps the sample to be tested stay more stably in the reaction chamber 309 after entering the reaction chamber 309, making backflow difficult to occur and preventing it from flowing to other chambers, thus ensuring that each step of the reaction can be completed smoothly until the final accurate detection is realized.

Preferably, the cross-section of the reaction chamber 309 is drop-shaped (FIG. 40), and the exhaust port 318 is located at the top of the drop shape, which helps the liquid paraffin to collect upward, making it easier to condense near the exhaust port 18.

The solid reagents pre-embedded in the liquid storage chamber 304 and the reaction chamber 309 can be flexibly set. Different reaction reagents are pre-embedded in different chambers. When the microfluidic chip 301 provided in this example is used for genotyping detection of the target nucleic acid, the liquid storage chamber 304 is used for amplifying the target nucleic acid, and nucleic acid amplification reagents are pre-embedded in the liquid storage chamber 304; the reaction chamber 309 is used for detecting different genotypes of the target nucleic acid, and detection reagents for different genotypes are pre-embedded in the reaction chamber 309. The liquid storage chamber 304 is used for amplifying the target nucleic acid, and nucleic acid amplification reagents need to be pre-embedded in the liquid storage chamber 304. The reagents used for lysis are added from the sample-adding port 306 during sample detection; the nucleic acid amplification reagents are solid amplification reagents prepared by freeze-drying or drying, including but not limited to spherical, powdery, flaky, or blocky preparations in the form of freeze-drying, drying, air-drying, etc. The involved amplification reactions include, but are not limited to, RPA (Recombinase Polymerase Amplification), ERA (Enzymatic Recombinase Amplification), LAMP (Loop-mediated Isothermal Amplification), NEAR (Nicking enzyme-Assisted Reaction), NASBA (Nuclear acid sequence-based amplification), HDA (helicase-dependent amplification), TMA (transcription mediated amplification), etc. The reaction chamber 309 is used for the fluorescent detection reaction between the amplified sample and the detection reagent. Detection reagents need to be pre-embedded in the reaction chamber 309. The detection reagents are solid detection reagents, including but not limited to spherical, powdery, flaky, or blocky preparations in the form of freeze-drying, drying, air-drying, etc. The involved detection reactions include, but are not limited to, various probe detection systems established based on RPA (Recombinase Polymerase Amplification), ERA (Enzymatic Recombinase Amplification), LAMP (Loop-mediated Isothermal Amplification), NEAR (Nicking enzyme-Assisted Reaction), NASBA (Nuclear acid sequence-based amplification), HDA (helicase-dependent amplification), TMA (transcription mediated amplification), hybridization probe technology, CRISPR, Ago, RNaseH, etc. The detection results include, but are not limited to, fluorescent detection reactions and test strip visualization detection methods according to different actual needs. When the microfluidic chip 301 provided in this example is used for multiplex detection of target nucleic acids, the liquid storage chamber 304 is used for adding sample nucleic acid lysates, and solid detection reagents for amplification and detection of different nucleic acids need to be pre-embedded in the reaction chamber 309, which specifically include detection primers, probes, and detection premix. The detection premix includes enzymes and buffers required for detection.

The chip body structure of the microfluidic chip 301 uses materials with low cost, easy processing performance, and good biocompatibility for chips, such as PMMA (Polymethyl Methacrylate), PP (Polypropylene), PC (Polycarbonate), etc., as the substrate. Channels and reaction tanks are directly made by laser etching technology, or structures are etched on the PMMA substrate, and then PDMS is used for reverse molding.

During the use of the microfluidic chip 301, it needs to be kept in a vertical state, with the sample-adding port 306 facing upward. When in use, the microfluidic chip 301 can be inserted into a matching detection device, so that the microfluidic chip 301 is placed vertically with the sample-adding port 306 facing upward, and the detection device can provide a heat source to heat specific positions of the microfluidic chip 301 (such as the reaction chamber 309, valve block 321, etc.); it can also detect the fluorescent substances generated after the sample reaction in the microfluidic chip 301 through the detection device and read the detection result.

The microfluidic chip 301 provided in this example, through the clever design of flow channel connection and chamber position, does not require additional driving. The liquid can realize the sequential and independent amplification and detection reactions of the sample to be tested according to its own asymmetric pressure. The whole process is easy to operate and has a fast reaction speed, and can effectively realize high-throughput detection of pathogens; in addition, the amplification/detection reagents of the microfluidic chip are all dry reagents, which can realize the room-temperature storage and transportation of the chip, avoiding the limitations of cold-chain transportation and −20 °C storage.

### Example 14: Usage Method of the Asymmetric Pressure-Driven Typing Chip

The asymmetric pressure-driven typing chip provided in Example 13 can be applied to various nucleic acid detection methods.

Method 1: Taking the genotyping detection based on enzyme-catalyzed isothermal amplification technology as an example, the usage method of the microfluidic chip device is illustrated:

Transfer: Add the sample to be tested containing lysis solution into the liquid storage chamber 304 of the microfluidic chip from the sample-adding port 306. At this time, tighten the tube cap 305 to seal the chip. The sample enters the bottom of the liquid storage chamber 304 and dissolves the pre-embedded solid amplification reagents.

Reaction: Heat the chip. After the temperature rises to a certain level, an amplification reaction occurs in the liquid storage chamber 304. The amplification reaction includes, but is not limited to, isothermal amplification reaction and PCR reaction; while the amplification reaction is in progress, the valve block 321 blocked between the liquid storage chamber 304 and the detection chamber 309 is also continuously melted. After the valve block 321 is completely melted, press or screw the tube cap 305. Driven by the instantaneous asymmetric pressure, the amplification product will move to the reaction chamber 309 pre-embedded with solid detection reagents to achieve pressure balance in the system. At the same time, the reagents in the reaction chamber 309 are dissolved and a detection reaction is carried out;

Read the result: After the reaction is completed, read the fluorescent signal through the device to obtain the detection result.

Method 2: Taking the multiplex detection based on enzyme-catalyzed isothermal amplification technology as an example, the usage method of the microfluidic chip device is illustrated:
Transfer: Add the sample to be tested containing lysis solution into the liquid storage chamber 304 of the microfluidic chip from the sample-adding port 306, and tighten the tube cap 305 to seal the chip.
Reaction: Heat the chip. After the temperature rises to a certain level, the valve block 321 blocked between the liquid storage chamber 304 and the detection chamber 309 is also continuously melted. After the valve block 321 is completely melted, press or screw the tube cap 305. Driven by the instantaneous asymmetric pressure, the sample liquid treated in the liquid storage chamber 304 will move to the reaction chamber 309 pre-embedded with solid amplification reagents to achieve pressure balance in the system. At the same time, the reagents in the reaction chamber 309 are dissolved and an amplification reaction occurs. The amplification reaction includes, but is not limited to, isothermal amplification reaction and PCR reaction;
Read the result: After the reaction is completed, read the fluorescent signal through the device to obtain the detection result.

### Example 15: Verification of Genotyping Detection Effect of the Asymmetric Pressure-Driven Microfluidic Chip

This example uses the asymmetric pressure-driven microfluidic chip provided in Example 13 (FIG. 35) for genotyping detection. The ERA-CRISPR detection technology (enzymatic recombination amplification combined with CRISPR detection technology) was used to detect Grass Carp Reovirus (GCRV) nucleic acids (GCRV, Type I GCRV, Type II GCRV, Type III GCRV) in tissue samples of suspected grass carp hemorrhage disease, and the qPCR detection method (commercial reagent) was used for comparative verification. The diseased fish samples were lysed to release nucleic acids using a sample release agent, and the obtained lysate was used as a template. The same amount of template was used for loading, and the asymmetric pressure-driven microfluidic chip device and the qPCR detection method were used to amplify and detect the samples respectively. For the ERA-CRISPR detection technology, ERA freeze-dried microspheres containing specific amplification primers were pre-installed in the liquid storage chamber of the chip device, and CRISPR detection system freeze-dried microspheres containing specific detection systems were pre-installed in the reaction chamber. The presence of corresponding pathogenic microorganism nucleic acids in the samples was determined, and the results are shown in Table 5.

TABLE 5
Verification Results of Detection Performance
of the Genotyping Microfluidic Chip.

| | | Asymmetric | |
|---|---|---|---|
| | Type of | Pressure-Driven | qPCR |
| | Microorganism | Microfluidic | Detection |
| Sample ID | to be Detected | Chip Device | Method |
| Sample 1 | GCRV | +Positive | Positive |
| | TypeIGCRV | +Positive | Positive |
| | TypeIIGCRV | −Negative | Negative |
| | TypeIIIGCRV | −Negative | Negative |
| Sample 2 | GCRV | +Positive | Positive |
| | TypeIGCRV | −Negative | Negative |
| | TypeIIGCRV | +Positive | Positive |
| | TypeIIIGCRV | −Negative | Negative |
| Sample 3 | GCRV | −Negative | Negative |
| | TypeIGCRV | −Negative | Negative |
| | TypeIIGCRV | −Negative | Negative |
| | TypeIIIGCRV | −Negative | Negative |

It can be seen from Table 5 that under the same other conditions, the detection result of the asymmetric pressure-driven microfluidic chip device in Example 13 is consistent with that of the qPCR detection method, but it is more convenient and faster to use, and is more suitable for on-site detection in the field and special environments.

1. A microfluidic chip for detecting a nucleic acid, comprising:
a reaction chamber configured for amplifying target nucleic acid in a liquid sample to obtain an amplification product;
a detection chamber configured for detecting the target nucleic acid in the amplification product from the reaction chamber;
the reaction chamber is in liquid communication with the detection chamber by a first liquid channel,
a second channel for receiving the liquid sample including inlet and outlet;
the detection chamber is in liquid communication with the second channel by a third channel;
wherein air exhaust channel with a first end and a second end, the first end of the exhaust channel is air communication with the detection chamber or/and the reaction chamber, the second end of the exhaust channel is air communication with the second channel.

2. The microfluidic chip according to claim 1, further comprising a sample-adding area and sample and a wash chamber; the sample adding area is in liquid communication with the inlet of the second channel, and the outlet of the second channel is in liquid communication with the wash chamber.

3. The microfluidic chip according to claim 2, wherein the reaction chamber is in the downstream of the sample-adding area and is upstream of the wash chamber, and the detection chamber is downstream of the reaction chamber.

4. The microfluidic chip according to claim 3, the chip comprising a chip body with a rotation center; the reaction chamber, the detection chamber, the first channel, second channel, third channel, the air exhaust channel are located on the chip body.

5. The microfluidic chip according to claim 4, the reaction chamber is closer to the rotation center than the detection chamber, and the second channel is closer to the ration center than the third channel and the reaction chamber.

6. The microfluidic chip according to claim 5, wherein the second end of the exhaust channel is closer to the rotation center than the first end of the exhaust channel so that a exhaust direction of the exhaust channel is opposite or at least partially opposite to the rotational centrifugal direction.

7. The microfluidic chip according to claim 5, wherein the chip body circular shape with a center and the rotation center is located in the center of the circular shape.

8. The microfluidic chip according to claim 5, wherein the chip body can be applied by a centrifugal driving device to drive the microfluidic chip to centrifuge, wherein the device can apply the chip with a first speed rotation and a second speed rotation.

9. The microfluidic chip according to claim 8, wherein the second speed rotation is higher than the first speed rotation.

10. The microfluidic chip according to claim 9, wherein the size of the second channel is bigger than the size of the third channel as that the liquid sample is filled into the second channel, and the liquid sample cannot enter into to the third channel without rotation.

11. The microfluidic chip according to claim 10, wherein when the first speed is applied by the centrifugal driving device to the chip, the liquid in the second channel can enter into the reaction chamber but not enter into the first channel.

12. The microfluidic chip according to claim 11, wherein the first speed is applied by the centrifugal driving device to the chip, an air in the reaction chamber is exhausted into the second channel by the exhausted channel.

13. The microfluidic chip according to claim 12, wherein the second speed is applied by the centrifugal driving device to the chip, the liquid in the reaction chamber can enter into the detection chamber through the first channel.

14. The microfluidic chip according to claim 12, wherein the second speed is applied by the centrifugal driving device to the chip, an air in the detection chamber can be exhausted into the second channel by the exhausted channel.

15. The microfluidic chip according to claim 14, wherein the air in the detection chamber is exhausted to the exhausted channel through the first channel.

16. The microfluidic chip according to claim 15, wherein the first end of the exhausted channel is directly connected with the reaction chamber as to be in gas communication with the reaction chamber.

17. The microfluidic chip according to claim 15, wherein the first end of the exhausted channel is directly connected with the first channel.

18. The microfluidic chip according to claim 15, wherein the length of the third channel is shorter than the second channel.

19. The microfluidic chip according to claim 16, wherein the reaction chamber comprises an agent for amplifying target nucleic acid.

20. The microfluidic chip according to claim 1, wherein the air exhaust channel is not liquid communication with the detection chamber and the reaction chamber.
